SEQUENCE LISTING (13 sequences)

SEQ ID NO: 1 - protein, 1624 aa, Homo sapiens
  REGION 357..586: note = TP2E fragment
  REGION 483..565: note = TP fragment
MPRPGTSGRRPLLLVLLLPLFAAATSAASPSPSPSQVVEVPGVPSRPASVAVCRCCPGQTSRRSRCIRAFCRVRSCQPKKCAGPQRCLNPVPAVPSPSPSVRKRQVSLNWQPLTLQEARALLKRRRPRGPGGRGLLRRRPPQRAPAGKAPVLCPLICHNGGVCVKPDRCLCPPDFAGKFCQLHSSGARPPAPAVPGLTRSVYTMPLANHRDDEHGVASMVSVHVEHPQEASVVVHQVERVSGPWEEADAEAVARAEAAARAEAAAPYTVLAQSAPREDGYSDASGFGYCFRELRGGECASPLPGLRTQEVCCRGAGLAWGVHDCQLCSERLGNSERVSAPDGPCPTGFERVNGSCEDVDECATGGRCQHGECANTRGGYTCVCPDGFLLDSSRSSCISQHVISEAKGPCFRVLRDGGCSLPILRNITKQICCCSRVGKAWGRGCQLCPPFGSEGFREICPAGPGYHYSASDLRYNTRPLGQEPPRVSLSQPRTLPATSRPSAGFLPTHRLEPRPEPRPDPRPGPELPLPSIPAWTGPEIPESGPSSGMCQRNPQVCGPGRCISRPSGYTCACDSGFRLSPQGTRCIDVDECRRVPPPCAPGRCENSPGSFRCVCGPGFRAGPRAAECLDVDECHRVPPPCDLGRCENTPGSFLCVCPAGYQAAPHGASCQDVDECTQSPGLCGRGACKNLPGSFRCVCPAGFRGSACEEDVDECAQEPPPCGPGRCDNTAGSFHCACPAGFRSRGPGAPCQDVDECARSPPPCTYGRCENTEGSFQCVCPMGFQPNTAGSECEDVDECENHLACPGQECVNSPGSFQCRTCPSGHHLHRGRCTDVDECSSGAPPCGPHGHCTNTEGSFRCSCAPGYRAPSGRPGPCADVNECLEGDFCFPHGECLNTDGSFACTCAPGYRPGPRGASCLDVDECSEEDLCQSGICTNTDGSFECICPPGHRAGPDLASCLDVDECRERGPALCGSQRCENSPGSYRCVRDCDPGYHAGPEGTCDDVDECQEYGPEICGAQRCENTPGSYRCTPACDPGYQPTPGGGCQDVDECRNRSFCGAHAVCQNLPGSFQCLCDQGYEGARDGRHCVDVNECETLQGVCGAALCENVEGSFLCVCPNSPEEFDPMTGRCVPPRTSAGTFPGSQPQAPASPVLPARPPPPPLPRRPSTPRQGPVGSGRRECYFDTAAPDACDNILARNVTWQECCCTVGEGWGSGCRIQQCPGTETAEYQSLCPHGRGYLAPSGDLSLRRDVDECQLFRDQVCKSGVCVNTAPGYSCYCSNGYYYHTQRLECIDNDECADEEPACEGGRCVNTVGSYHCTCEPPLVLDGSQRRCVSNESQSLDDNLGVCWQEVGADLVCSHPRLDRQATYTECCCLYGEAWGMDCALCPAQDSDDFEALCNVLRPPAYSPPRPGGFGLPYEYGPDLGPPYQGLPYGPELYPPPALPYDPYPPPPGPFARREAPYGAPRFDMPDFEDDGGPYGESEAPAPPGPGTRWPYRSRDTRRSFPEPEEPPEGGSYAGSLAEPYEELEAEECGILDGCTNGRCVRVPEGFTCRCFDGYRLDMTRMACVDINECDEAEAASPLCVNARCLNTDGSFRCICRPGFAPTHQPHHCAPARPRA

SEQ ID NO: 2 - protein, 18 aa, Mus musculus
EPRPRPEPRPQPESQPWP

SEQ ID NO: 3 - protein, 17 aa, Homo sapiens
EPRPEPRPDPRPGPELP

SEQ ID NO: 4 - protein, 18 aa, Mus musculus
ESQPRPESRPRPESQPWP

SEQ ID NO: 5 - protein, 19 aa, Homo sapiens
EPRPEPRPDPRPGPELPLP

SEQ ID NO: 6 - protein, 43 aa, Homo sapiens
FLPTHRLEPRPEPRPDPRPGPELPLPSIPAWTGPEIPESGPSS

SEQ ID NO: 7 - DNA, 155085 bp, Homo sapiens
  source 1..155085: mol_type = other DNA
aagctttatattataaatacaaagtaaacatgaaaataaaacccaaacatagcagtgttattaaactctggcctgtagcagtggctcacacctgtaatcctagcagtttggaggccgagacaggtggattacttgagacctggagtttgagaccagcccaggtgacacagcaagacctcatctctactaaaaataaaaaaaaattagccaggtgtggtggtatgcacctgtggtcccagctacttaggatgctggagtgcgaggatcgcttgagcccaggaggtcaaggctgcagtgaactatgatcactcattacaccccagcctgggtgacagagcgagatgctgtctcaaaacaaaacaaaacgaaaaacaactctggctagatgctattgcttgccaagggtgcagtcttccatttattaaaagtgaaaattagggccaggcacattggctcatgcctgtaatcccagcactttgggaggctgaggtgggtggatcacctgaggtcaggagttcgagaccagcctggccaacatggtgaaaccttatctctgccaaaaatataaaagattagccatgtgtcgtggtgggtgcttgtaatctcagctacttgggaggctgaggcaggagaatcacttgaacccaggaggcagaggttgcagtgagccaagattgtgccattgcactccagcctgtgcaacgagcgaaactccaactcaaaaaaaaaaaaaaaaaaaaaaacggaaaattaggtaatactgcagattcattaatatcacagcagcactaaactcagtcttcctctgtgctgggctcaagggaactgaaagggaagtaacttgctatgtggttcagtgtcatttcatgctgtgtcactgcaccacttaaaatcttcacccggagactatgtcccacacatggggaaatcaatcaccgtcatattttaagtgcttgctgtgtgacagccacgattacaatgactgttactctacctcttattctcactgtagtgttcccagtgaagcggcggaaccggcacagcctagtggggcctcagcagctaggaggacggccagcccctgtccgacggagcaacacgatgccccccaaccttggcaatgcagggctgctgggccgaatgctggatgagaaaacccctccctcaccctcaggtacgtttctatctcgtggaaggttggggcagctgtggggtccctgtgaccctcttctgatccctactcttcccctcaaggacaacctgaggagccggggatggtgcgcctggtgtgtggacaccataattggatcgctgtggcctatacccagtttctagtctgctacaggtgcttggggagggagtggcaggaggtcccagccctgttgatgggaagggcatgcggtgagggttggtgatgtcccctcgatcctacaggttgaaggaagcctctggctggcagctggtgttttccagcccccgcctggactggcccatcgaacgactggcgctcacagcccgggtgcatggtggggctttgggtgaacatgacaagatggtggcagcagccaccggcagcgagatcctgctatgggctctgcaggcggaaggcggtggctccgagataggtatgaccccaagccttttccagaaccctctgtcctttaaattctactgcctgttaaaatgacccaccccataaaatcctcaaacccataagaattctctgctctattagatctgtttgtgtcattagaatgctccagcccttcagaattctgtatcctgttagaattccctggcttcctagaaaaatccacccacatacaattctccctccatcagaattccctgccctgttagatctcttggttttgttagaatgcgctattctgtgtggccatgagaaatgtctgacttggcccggtgcggtggctcatgcttgtaatcccagcactttgggaggctgaggcgggcagatcacttgaggtcaggagttcgagacccccttggccaacagggtgaaacccagtctttatttcaaaaatacaaaaaaattagctgggtgtggtggcacgtgcctgtaatcccagctactcaggaggttaaggcaggagaatcactggaacctgggaggcagatgttgcagtgagctgagatcgcaccactgcattccagcctgagcgacagagtggacagagtgagacaccatctcaaaaaaaaaaaaaaaacaaaaaaagaaatctctgacttatggctgggcacagtaactcacatctgtagttccagcactttgggaggccatggcaggaggattgcttgaggccaggagttcgaggctgccgtgagctatgtaagccatgattgcaccactgcactccagcctggacaacagagcaagaccctgtctcaaagccacagcaacaaatctcggacttattaatctgtccccaaataaatctcagccctcgtaagaatcccctgccaccagtggaggtttttttgccttattgcaaatctcagtgctgttagaattccagaccgttttagaatcccataccccatcagaaatcttctggcccattttatcttctctaattcctgctggaatcttcactttttttttttttgaaatggagtcattctctgttgcccagattggagtacagtggtatgatctcggctcactgcaacctctgcctcctggggtcacgcaattctccttcctcaacctcccaagtagctggggctacagttgtgtgctaccacactcagctaatttttatattttttagtagagatggagtttcatcatgttggccaggctggtctcaaactcctggcctcaaatgatccgcccacctgagcctctcaaagtgttgggattacaggtgtgagccactgtgcccagcccctgatggaatcttctatccttcaaggattctgtggtcccactgaaccctctggtccttggagttcttcctcccacctctcctcctccatgaggcagggttccagctgagccctttttgcccactgcaggggtctttcatctgggggtgcctgtggaggccttgttcttcgtcgggaaccagctcattgctacaagccacacagggcgcatcggggtgtggaatgccgtcaccaagcactggcaggtcagagttctggccagcctggctgcccccttcccagttttccagactgtacagacccaggaaggggttcacgtctgcccccacccccaccccctaagcctggaggaagccagagaaccagcagctggccagagcctggggatgtctgtgataaaggcctaaggagcaccagggaagccttcctggaggaggtgctgcagagcccacatgaagcatgaatgggtttgaaaaggagtagggaggattgtcttgggactgggtgagagagcaggttaggggggatgctgtagaagacaagaactggcttggctcggtggctcacacttgtaatcccagcactttgggaggctgaggtgggaggatggctttgggagaaagacagacagaggcagagaaagagacagagaggcagaaaaccaggcaaatagaaatagaaaatcagaaagaggtgacagagaagggtagacagagtcaaaccagactgaaccactgtggcagatgggggaactcagagagataaatggagatgaattagagtcatgtcagaagtacatagtggaggccgggtgcagtgactcccgcccgtaatcccagcactttgggaggccgaggcaagaggatggcttgagcccaggagtttgacgccagcctgggaaacataataagactctgtctctacaaaaaatttaaaaaattaaaactttaaaaagtacagagtccaggagaggccaagagtcagacagacagatgcatcaaagggaggaagggagggaacaagacagggacaaaggcaggcctggggtggacggggctggggagcagtgggtgggttctggaggtggaggagagggcccatcctctgtgaggggtcaccacggaaggaaaggaatggaagggctcccggctgcagcctgtcctgccccacccccaggtccaggaggtgcagcccatcaccagttatgacgcggcaggctccttcctcctcctgggctgcaacaacggctccatttactacgtgggtgagcagcagcctgtgtcccgggtgcccgagaccctcccctgggagaggggaagggagggagagaggctggggcacttggaacaaacccgctccaccctccctgcccaccccagatgtgcagaagttccccttgcgcatgaaagacaacgacctccttgtcagcgagctctatcgggacccagcggaggatggggtcaccgccctcagtgtctacctcacccccaagaccagtaagctatgacccggcttcccctgctgctgtcacctgccagccccagccccagcctgcagctgtcccccaacttgtgtagcatgacccagaggggtttatcgaggggcggcagattcaactctctctcatttttttttctgagacagactctcattctgttgcccaggctggagtgcagtggtgcaatcttggctcactgcaacctccacctcctgagttcaagcgattctcctgcctcagcctcccaagtagctaggattacaggcgcccactgccacgcctggctaatttttgtagttttagtagagatggggtttcaccatgttggccaggctggtcttgaactcctgacctcagatgatcctcccgcctcagccccccaaagtactgggattacagacgtgagccaccgcgcccagccagattcaactctcatccagtcattcaacaaatatttactgagtatccctgtatataagcacgttctaggtgctagatggtggccaccctctccactaggggcagaatggaccattaggagttaatctttgtgtttctttcccacctttgtgattaaggcatgatgttcacatgttcataaaacaacacagatgcaggttttacagcagatgaacctgagttctcttacttgagacagttttttgttttgttttcttgacatcaaatgcagaatgggttaatttttgagacgtaattctcataccataaaatttcccatttgaacgtgcacaattcagcggattttagtatattcacaaggttgtacaactatcaccatgaacaaattccagagtatgttcatcacccccgtaagaaaccccctgatctttagcagtaactccacatttctcacttctgataccctctagaaatcactaatatgctttctctttttgtttgagacaaggtctcgctctgctgcccaggctggagtgcagtggcatgatctcagctcactgcaatctctgcctcttgggttcaagcgattctcatgcctcagcttcctgagtaggggaggattacaggtgtgcatcaccatgcctggctaatttttctactttaagtagagactgggttacgccacgttggccaggctcaatatgcttcctatacctatggatttgcctgttctggacaattcctgtaaatgggatcatacattatatggtcttttgtgtctgcatttttcacttagcatatttttttaaatggttagtgttttctgggtcttaagatatccttacctattctaggttacaaagatcttctcctgaatttcttctagaagatttacagtttagtttttacatggaagtctgttctatcagaaattaaattttatgtatggtgtgaagcagggattattcttttttaaaaaaaaatagagacagggtcttgctctgttgcccaggctagagtgcagtggcatgatgtgagttctctgccacctccgccactcgggctcaagcgattcttctacctcaacctcctgaacagcagaaccacaggcatgcatcaccacacctgtctaatttttgtatttttttgtagagacagggttttgccatgttgcccaggctactcttgaactcctgggctcaagtgatcctcacactttggcctccaaagtgctggaaatacagacttgagtcactgtgcttggccatagctgctgctgctgcttttcttttttttttaaagcagaaataagttaataatacacctaacttcatgacacattatcacctttcttctttgtaaggcccttttcttatttcttttttttctttctttctttttttttttttttgtcttttcctgagacagagtatcgctctgtcattcaacccaggctggagtgcaatggcgcaatctcagctcactacaacctctgcctcctgggttcaagcaattctccagactcagcctcccaagtagctgggattacgggtgcctgccaccatgcccagctaatttttgtgtttttagtagaggcagggttttgccacgttggctaagcaagttttgaactcctgggattctcctctcgtcgtctcccaaagtgctggaattacaggcatgggctgctgcacccggcctctttcttatttcacttacttactcatttcctcctcatctcccttcccgccacatgccactattatgtcatttttctttgttttttgagacagtctcgcttcgtcacctaggctggagtgcagtggcgcaatcatagctcactgcagcctcaccccctgggctcaagtgatcctcctacttcagcctcttgagtagctgggatcataggtgaatgccatcacacccaactagttttttaaaaatcttttgtagagatgagggtctcactatgttgcccaggctggcctcaaacgcctggcctcaagcaatcttcctgcctcggcctctgttctctgttttactttctctttcctctcttatttgctgtttcactgttacaatttctgtctctgggcctctcctcctttgacctgtgcctttgtctccctgttcctccatttcccacatctcatcaccatctttttgtgtccttccctctctccttgctttctgactgtgtctctgttctgtgtctctttctttctttctttctttttttttttttgagacagagtcacattctgtcatgcaggctggagtgcagtggcgtggtcttggctcattgcaacctctgcctcctgggttcaagcaattctcatgcctcagccttctgagtagctgggattacaggtgcgtactaccaccaccagctaatttttgtatttttactagagacagggtttcagcacgttggccaggctggtcttgaacgcctgacctcaagcgatccacccgcctcagcttcccaaagtgatgggattacggcgtgagccaccgtgcccggcctctccttgctttctgactgtgtctctgttctgtgcctgtttctttctccctgccctggtttctgtctctgtggcccaggccctctcctcatccttggcccctcaccaggtgacagtgggaactggatcgagatcgcctatggcaccagctcagggggcgtgcgggtcatcgtgcagcacccggagactgtgggctcggggcctcagctcttccagaccttcactgtgcaccgcagccctgtcaccaagatcatgctgtcggagaagcacctcatctcaggtgagcctctgtggggtgctccagtgctgggagagacagctcagatgggagtgtggagacaggaggatgagaatttccactggggagggacattgtgtaggaagagagctctggcggagaggattgacctgggatgggactgtaattgcagaagtgagagaataccataggggatcagtctggggaagagtgaatctgtgtgtgtttgggaatccatcaattcagcaacgatttacggggcacccaccatgtgccaggcataggggatacagtgatgaacaagacaggccaagtccctgccctcgtggagctgacattctagcagcgatggcagacaataaatctataaaaatggacatcaatggccagctgcggtggctcacacctataatcccagaactttgggaggccaaggcgggtagattacttgaggtcaggagttcgagaccagcctggccaacatggtgaaaccccacctctaccaaaaatataaaaaattagccaggtgtggtggcacgtgcctgtaatccccgctactcgggaggccgagggaggagaattgcttgaacctgggaggcggagattgcagagagcccagatcatgctactgcactccagcctaggcaacagagcgagactccatctcaaaaataaatagataaaaatgcaaatcaggctgggcactgtggctcatgcctataatcccagcactttgggaggccaaggtgggaggattgcttgaggctaggagtgtgagaccaacctggacaacaaagtgagacattgtttctacaaaaaatttttaaaaaattagccaagtatggtggcatgcacctggggtcccagcttcttgggaggctgaggtggagggatcacttgagcccaagagtcaggggctgcaatcagctatgattgcacccctgcactccagcccaggcaacaaagcaagactctatctcataaaatattaataaaataatctaaatcaacaccaggcaatgataagggctatgaataaacatgaagctgtgtgtggctagagagtgtcaagtcgctggagaggggtcattcagggaaggcctgaggaaggacctttgagcagaaataacagaatgagcagtgcagattggtagggacaagcattcaggccgagggaacaggcattgcaaaggctctgaggtgagaggatgcctgaggaagtgcagggagccacttgtgcctggaacagagtaaatgagggggagaatgggaggagatgagggtatggaggtgacaagggcaggagccacagagggctccgagcaggggagggacatgactagcttcaggtgttcacaggctccacctggctgcccgtgggagcacactactgggataggacaggagcagggacctctgggaggggcgtgctgatacggtccaggcaggtgacgaaatggaccaggaccggtgtggggcagaggatagggcaaggtgagatcaggctctggaacagttttggaggcagaggcaacagggtttttaacatttggaattctggggaggaaaggattctctgaaagggaagaactcagcaagggctgaccctcctgaaacggggctgtcctgaggctctggacgggcaggcagcattgatgcctgcaccaggcctgacttcctgctctccttgtgcccctcagtctgtgccgacaacaaccacgtgcggacatggtctgtgactcgcttccgcggcatgatttccacccagcccggctccaccccactcgcttcctttaagatcctggctctggagtcggcagatgggcatggcggctgcagtgctggcaatgacattggtgcctactggctcctggccttcccaccccactgacccccttacctgacccctgttgacctccgctgattcccacttgcggccacctgacccaggagcctgcaacgattggggtgtcctgatccttgaggcacctgagctccactgactgccacaaccccccagccctctctggccctgcccaactctaacaacctggggatgggactggggtcttggggtggtgggtttcatcagtggggaaggaagtctgaggctgagggaatcccagctggccctgacccctgcccctgcctggcccaggcccctacggtgagcgggacgaccagcaagtgttcatccagaaggtggtgcccagtgccagccagctcttcgtgcgtctctcatctactgggcagcggtgaggacagtcctgtccaacagggagggaggacagtcctgtccaacagggagggaggtcaggggagaaggcaagatgctagccagatcactgaggccagaaaggggtcccagccaaggagcattgaaggagcaaaggctgggagggaggacaggcctggtaggtttgtcttctggtatcaaaggtgtgacaggtgttggtgggactgacctggcgcagtgccagcccagtgcaggcagggccaggttgaaggcacagggggtggggtcagggtgggatggggggcccaggcggagggggaggggtgggatgggggcccagggggagaggtcggggtgggatggggggaccagggggaggggttggggtgggatagggggaccaggggcaggggtcgggatgggatggaggagacccagggagctgtaggagccttgcttcagctgggaaaggggcctcggaggatgttctggatgaagggactctgagcagagacctagaggacaagtaggaaggagcagaagaaagaaggcaggggaagggggccccagcctgagggaactgtatgtgcagaggctgagaagggccaaatagggtcacatgggaaggacacgtgggctcctgggctggagcacagaacaggaggtgaggatggggtgcagatgcgggcgggccttgtcaggctgtggaagggctgaggctttgtcctgtgcggggagccgtggccactgcatgggggatgaaaggaggcttgatgggaggccaggagcccagggaggaggccaggaggctgataggaggtgggggagcagctgggacgggcctcaggtcttccctgcagtgggagcagggaagagcagaagctctgggagactccctgggtgtttggggctgcggtgtccaagagctgctggacccttgggactggaactcaaaagcaggctagggatggataaagattgggatggcccctggagaggcaggaactgcagccacagtggtggggactgggacgaggaagggtgagaaagcgagggtgaccacctcccccactgcaccccccagggtgtgctccgtgcgctccgtggacggctcacccacgacagccttcacagtgctggagtgcgagggctcccggcggctcggctctcggccccggcgctacctgctcactggccaggccaacggcagcttggccatgtgggacctaaccaccgccatggacggcctcggccaggcccctggtactccctgccccaccccaatcccgtcccaagccccacagcctcacccagaaccactctccactgccaactgcttgatctctctcccaggcccttgccctctgaccccttttcctttgaccccctctctgctccccatcccttcctgcccttgtttttcaacccctgtctcagcctctggcccccatgcattgatctctgcttcctctctcctgtcctgaccctcggtgcttgcactgcaatgcaacccaggcccttgccctatgacccctgtcttgccccctgaccctgcttccgtgccccccagcaggtggcctgacggagcaagagctgatggaacagctggaacactgtgagctggccccgccggctccttcagctccctcatggggctgtctccccagcccctcaccccgcatctccctcaccaggtagccacaactccactgccccttctgtgcaatgaggggagaggggacagcatggtgttcccggggacagagtggcccagctgccccgtgatgacgtgcacttactgtccttacttcctcagcctccactcagcctccagcaacacctccttgtctggccaccgtgggagcccaagccccccgcaggctgaggcccggcgccgtggtgggggcagctttgtggaacgctgccaggaactggtgcggagtgggccagacctccgacggccacccacaccagccccgtggccctccagcggtctcggcactcccctcacacctcccaagatgaagctcaatgaaacttccttttgaacaacgcagctgccatgatgccttgggatgccctggtcctgggggactcaggtgcctccctgattcctgtgggaaccccgggttcagggccagggcctccttggaataaatggttattgttactaggtccccaccttccctcttttctggaagccaaagtcaccctccccaataaagtcctcactgccaacatcttgcttattcttagcaggtttgggactccccaggaaaggggtggggaagatgtgggacccaacccgatatatgcaaaatcaggaacatctacctctcttgtttaagcaaagggaggccaggcatggtggctcacatctataatcctggcactttgggaggccagtttgcttgtgctcagaagtttcagactagcctgggcaacataacaaaaccccatctctacaaaaaaatacaaaaattaagcaggcatggtggtgcatgcctgtggtcccagcttcttgggaggctgaggtgggaggatcgcttgagcctaggaggccaaggctacagtgagccatgatcacaccactgcactccagcctgagtgacagagtgagatcctgtctcaaaaacaaaacaaaacaaaaaaggtgttgcaggggaggaatgtgttggcttttactctgccttcaggcatggctggatccggggcttcaaacactaattcagaatatctttttttcttttttcttttcttttttaaagtctcactctgtcatccaggctggagtgtagtggtatgatctcagctcactgcaacctccacctcccgggttcaagcaatcgcaatcctcctgcatcagcctcccgagtagctgggattacaggtgtgcaccaccatgcccagctaatttttttatttttagtagagacagagtttcaccatattggccaggctggtctcgaactcctgacctcaggtgatctgcccacctcagcctcccaaagtgctgggattacaggcgagagccactgtgtccggcccagaatatctttttatctcctaacttcacttacctctggaatacatcttcaatcaggttttctctctgtggcaaggtgactctaagcagctgcttagcaactctttcttccctagcaacagcaaaaatcacaagactgattttcattggcttgaattaagtcaggtggccattcctgaaccaattactgtggccagtgtgatgaaggccttccattattgcagcctggatcatgtgtcctgctgtatttttagtagaggcagggtttcaccatgttggccaggctggtctcaaactcctgacctcagatgatccacctaactcggcctcccagagtgctgggattacaggcgtgagccacctttcctggctggccttgggattttttttccccaaataatgcccgttcctacggcacttccttgaggagattttctgcatcttttttccttgtgccaataaaaaagggacagaaagtggggacagactaggagtagagaatactagaacatgctaattctagaattccatagtttctggaatgccagagcttctgaaacatagtagagaggggtctcgaacattaggacatctcggacacaaggatattctggagtgctcatggattaacaatattagagttctcccctgcttatcagagaatccagaatatagaagtttctgacaaaaaggacctgaaaaatcactgattctagaataggggagtgtcaaggggagttcatacccatgaatctccaccggtaggaatatcttgcatgccaggatgtctggaatgctgagaattataggatacagaaaggaacctggattactaggaaatctagagaatgggagagatcctggactatccgcccttccagagcactggaaggtttgtttgcgttccaagaaatccataatggcaggagattccggattatcagagaatctggaacacaaatgggagacgtaaaaggtgagtgcctctgaaaccggggtgttctgaaatagctgtgcacctcaaaccctaattttgtttttgtttgtttgtttttgagacagggtcttgctttgttgcccgggctagggtgcagtggtgcaatcatggctcactgcagcctcggccttctgggctcaagtgatcctcctgcctcagcctcctgagtatttgggactacaggtgtgtgccaccacagccagctactttttaagtttttttgtagagacattgtctcactatgttgcccaggctggtctcaaactcctgggctcaagccattctcctgccttagcctcccaaagtgctgggattacaggtgtgagctaccatgcccagcctcacactctagaaaattttttacatgccaggatattcagaatattgggggatattggcatgttaggaaatctagtatgtagggggagggcctggaaggtcaggaaatctagaatatggtggtgttttgtaatgctcacagatctagaatattagagtcatttttttttccaggccggagaatccagaacgctcgtgttgctgaaatgctggtcattctattcttatttcatgtttaaatttttattattttctttatttcttttttttgagacagggtctttctctgttgcccgggttggagtgcagtggtgtgatctcagctcacctcaacctctgcctcccgggttcaagtgatcctcccaccttagcctcttgagtagccgggattacaggcgtgcgccaccatgcctggataatttttgtattcttagtagagacagggtttcactgtgttggccaggctggcctgaaactcctgacctcaagtgatctgccctcccacctcggcctcccaaagtgctgggattacagacgtgagccggccttattctgggggagggggtggggggggagagagagagagggagagagagaggagaatttgggatgttagcaaatctagaacactgaagagctgggttttttaaatgctagagatagtattatcctagtatagtattaactctgtgtgtttttgtgcgtgagagggcatggggggaggggtggaagattctggaatgcggggggactagaactctgagggattctgaagggttgagatgagttaggattcaagaggtgacatagacacgatcctgggagatagtgacactgatgggagggatggggagctggggactccaaattctagggggaggggaggctaacgccggaagttgcagtggtgagccagaggatgttgcctagcaacaagcattgaagatgcgctggatcccggacagaagggagaaatttcagcagcgcccccagcaaatgctccagccacagggtaggaggtggtgaggggggaagcgggtagggatatgctgtcctgcagggtcatgtgagcaggggtcttgggggttatacaaaaacagggtcatggagtacagtcatggggcagagccgggctctgggacagcccacgaatagctgtgaatggagtgtgagtgccgtcgtcacctgccccagggactgtgtgtccctggacggtgctggacccagatttctgggtgtcagtgtgggatggaggcatggattgctgtaggtggcagtaatggtgtgtagccgtgtgagtcagcctgtgtgtctcacagtgagcgggggtgaagggcttccccgtcagtcctgcacaccctcctggattggaagaaggagcaggagtggggggtggggggtgcaactggatcagttgccattcttggtctggggtgggctctgtggtcacggcgtgtggcctagtaccctgtgtgggtggagcctgggatccacgaggtgggggcggggctaacgctacagaactgggaagcttaagaatccaactgctttgggatatggggcctggaaccccaaagcgaagcaaacatgaaaggggtgggcttgggatcctacatgtggtcacgtcccagttttctgtgggaaagggtgtggcctgaaactctggaaaaagaggtgattaggggcagagtgaagctctgagggtggtgccttagggagagaaggggcttgtatttcagagaatatagggtggagccttgaatttataatgatcttctcaaagaattgatcccttctcaaggccctaggcttcaggggttccaggctcaccatctgcaaaagcttgctccccgaagctctgtccctcagtgaacccagccaggctctgcccctgacccacaggatctgctcccagggggtcctgggggctcctggctctgccccctcctgcacctcacttattgccatccccagagggtatcagacccccttccttgcctgagtctttgaatgctcccaagagctttgagtctcaagctgggcttgagaccctgagtttgaaggcttgaagcctggaaccccggtgtggacggggtttaagggcccctgtggcttgcagctcctagtggtggagactcttaggcaaggaagggatcggtactctcttttatatagttttatgtatttatttattattgagatagggtctcactctatcgtccaggctggagtgcagtgcagtgcagtggtgccatctcaactcactgcagcctccacttaccaggctcaagtgatcctcctacctcatcctcccgagtagctgggactactggctcatgccaccatacccggctaatttttggatttttttttttttttttttttttttttttttttgagacagagtcttgctctgtcgcccaggctagagtgcagtggtgtgatcttggctcactgcaacctccacctcctgggttcaaacgattctcctgcctcagcctcccgagtcgctgggattacaggcgcccaccactgcacccagctaatttttgtattttttagtagagatggggtttcaccatcttggccaggctggtctcgaactcctgacctcatgatccacccgcctcggcctcccaaagtgctgggattacaggcatgagccaccgcgcccggcctaatttttggatttttaatagagatggggtcctatgttgtactatgttgctcaggctcacctcaaactcctggactcaagcgatccacctcggccttccaaagtgctgggattacaggcccgcgccagggcacctagccggaactgggaactctctaaggagaaagtctgcaattctgggtggtgggaggctgctcttgggagcattcaaagactcaggcaaggaagggggtctgataccctctgggggcggcgataagtggggtgcaggagggggcagagccaggagcaccctgggagcagagtttgtggatcaggggcagagcctggctgggttcactgagggacagagcttcggggagcaagcttttgcagatggtgagcctggaatccctgaagcccagggtcttgagaagagatcaattctttgagaagatccaatgtgaacttgggaccctcagaggggcaggacctgggggctggtgcagggttggtgctccatttgcagaatcctcagtctctctcgtggaaaggggtacccgtggggggcttccagcttctacgccagcgagggagctaaattctgaccgaattctcccagaactgggagcagagagctgaggactgccagatggagtccagcttaggggaccctgcccccaaccccgctcccgtatccctcccatttttaagcacccctacgcctgggacctctgttccacgagaaggttcacggagccagtatccgagtcctaatttccccccaccgcgtggggaactcgaggtcccccgcgcccctagtccggcacctgtccctgctgcctcctgattggccgcgcggcgagcgctggcggcgccgggctgtgattggcgggaagttcgcagcccgtggctggactgtggagaaagtgagtcggcctcgggcgggggcgggggcgggggcaggggcggggccggggctagcctgacccgctggacagaccgcccgcccggagcgggactcggcgcccgacacgatgccgaggcctggcaccagcggccgccgccccctcctgctggtgctgttgctgccgctcttcgcagccgccacctccgccgccagccccagccccagccccagccaggtcgtcgaggtcccgggggtccccagccgcccggccaggtaagccccctgcttccttgtgccccttccacccaataagtctggtggtccaggaggaccacggctttcggattcaggggaaactcgaacccactgcttcgatgtggagatgcaaagagggcccttggatttattaggcgccctcaatttgtaggggttagattcggggagtactgagatttttaggggaaagagtcccctttattgtggtggagaaggctaagtctccagagaatcctggggttagttcggggaaaggtctctagtttattggggcagggggtggggggctgagctctcctagaatgatagagacgtgtggggtcttcgagctaggatggagcgccccaagtttcttgggcgcgtaaggaggaaaggtgttgggggattctggagggactttatgaggccgctgagttgtcctggttttctaggatttgggggctctgtgttcatgaaaagcgacctttgggtttttcgaattggtcaggaagacatagactcttgcgttgatggtggaggggcttgtggggagtggggtcgggtgggggagtcctcaggcttcagaacggttggggatgctcaagtttcctagaattagaggactctgggattgaatgaggctccagtgatttcaccgcgagagatccaggattccctatagatctgggggctataggcgcccttcctgtctgcgggtgacgggactgaaggaagccgtctgcccagcacccccacccaccctccccgcctggcggcccagcgctccgttacaaaggccccgggctcctctcccgcctgggtctctgcgaatgcgtttagtaacccgagccgcggcgggggcggggccgggaaggggttaacctggagaaaaggcgggagggatgacggcaagatcggggatccccgaggctccccaccagatggcctcatcctccactcggccccacccacctgagaggggcccacgcgctggacacccccctctctgcacccccccactgctctcctctcggggtctgggcgcgcgccctcgtctctagacccccttagcctggggataatggacttgtctggggggccccttcattgcgacgcacttgaaccggagctggaagctccgccactgagaccctctgggctgggagatgtctgcaagaacggcacctttctcctggtcaggcgggaaactgaggcccaaagaccaagaaacgctggcttctggacacacatcgtggaagggcaagaacgggctagaactcaggctctgggattctcagtcctggactgtgactcccgttatccccctccacctcagcaccctgctggacccacagtcagagggaagagccttccctccccagctgccgaggcctgtgaaaatggcgccacctgtcggccaggctcaggatggcgtctgagtcccggaccgggatgtgcgggagggcggggttctagattcccagacagtgcgggcttagaaatgctcttagaaatcgagtcttttgtttttgtttttgttttttaatcacttttaaaaggctttaaaataacgtattttttcaataaaatcgtacgccaaaatctcttcataagacaaaagtacggtttctgtgcccttcagcccgaagtctaagccaatttgcctcccattgtacagatggagaaactgaggcgtggggagaagaacctcgttagtcatatacctgtttcaagccccctcaggatgcgtcttggtttcccctccccgattgccctcctttgcagagttctggggtgctaggggcctgagtgacttccactccactatttatagcgttgctgtttgtcgctgctgcccgggccagacgtctaggaggagccgctgcatccgaggtgggttctggaagatggagtatgagtagggggcaaaagggaataggaggagaggggcagggagctccccatccctcccctcatcccagtgggagagctggatgcatttggtagagaagtacttggtcccctccttccccacttagtccctggctgtctccgaagccttctgcagggtccgaagctgccagcccaaaaagtgtgcaggcccccagcggtgcctgaacccagtgcctgcagtgcccagtcccagccccagcgtgaggaagagacaggtgtccctcaactggcagccactgacgtgagtgggcagtccctcccctaccaaatcctccctcaggaactcctagctttgcccccgtttccgctcctcctcccttcccctctccctctctctcccccctccctccccggctctttcttttgctatcagcctgtctgtccgtttctatttcatcccttgcctgcctatctcaggctcttcccatctctgcattatttctgtctctttctgtttctttctagttacctgtctcttttcctctctctctttctccctgtgtctcactgcctgcctctatgtctaagtctctctgccccttctctctttctctcccccaacccccatcatctctctttctctcagggtctttgtttctctcccactagaggcctggtccccataaataggtgttgtggggggagggttgggcagagccgctactgaaagacattttcctggcatggaggccctttggtggggaggtcagtttaacccggtgacagcagcttttccgtcctctcccacccaggctccaggaggccagagctctactgaagcggcggcggccccgggggccagggggccggggactactgagaaggaggcccccacagcgtgcccccgctggcaaggccccgggtaagcacatccgcctttcctccgcctggggcgaggggggttccgggccagataaagccgtctggttcccacggtccagccgccgcctacccgccccccgttgtagctcagcacccacccccaggacgaggccccctctagggaccccagcgtccggccgtgcgaggtgagcccgggacaggaccagacacaggccagaggcactgacccgcgggggtggggccgggaagccaggcgtccgggctccccgcggccgaagagggagggcgctggcccgttagaaagctccgccgaaccagcccagacaacaaaagcgattgtgcgggttggcgcctgcctgggcaccaaatgctcaagattgtggggcggctgggggccaaaggcctcctcctgtttcccgaatcttccccctgccctcctcccgctccagctcttgacccctagtctagaacacccctcatctcaaacaccctcaaaaccctcctcttgctgtagccttcagcccagatgtgacccctccccctaaagtcttccagatgtgagcctcgggccagatgttagtacccccgcccacagcctccctttatccattgtgacttctcccgccaggtgtggcgttctggccagatgtggctacatcttcacatgtatcccccccacctccagcctcctgcccaggatctagacgcccaattcctttatgtgcccaggtgtagcgacccttctcccatccctcagatgtcccacctggctgtccagatgtaccatgatagccccctatcaagatgaagcccctttctctgtgccccgggcagaagaggatcaactcttagctgtctccccaaatgtggcccccactactcttaatggatttcctctttatggattcagctctgtctggggggaaacccgcccgcccccacctgttcccttccccgccgctcccctacaggcccggtccacttgttggtgcgcggatggggacagagggggcgccccgctcacccctgcgagtctagcgcccagcctgcgagcctctggctaagcgcccagccccacgcgcccccgcgcggccgccggggagggagtggtgaggagggggggcctgagcgggggcgcgggcgacctcccccgcgggcgggcgggcgggtgcggccgggcccctcgggcgggctggggcggcggcgcggcggagcgcggcgctgcagccatggcgggcggcgtgcggctgctctgggtgtcgctattggtgctgctggcgcagctagggccgcagcctggactgggccggctcggagagcgtctccgcgtgcgcttcaccccggtcgtgtgcggcctgcgctgcgtccatgggccgaccggctcccgctgtaccccgacctgcgcgccccgcaacgccaccagcgtggacagcggcgctcccggcggggcggccccggggggacccggcttccgcgcctgtgagtgcggggtggtggtcccgagagagcggctccgggggggaggaggatccctggggatggaggagacagtcctccagggagagggagcccagattccatatgcaatagtgcaagggaatatcaggggctatttgaggaagggggccccaaattcctggcttttggggttgggtcctgagggttccgattttgcaagcaatgagggttcttagaggcctgtgatgtcccacgtgagggaattggcgccagctttcatgctttgcgtcaaaatggggatctcagaattgaggggcctgagaacttttgggaggggggctcgaattctgagacgctgagtaaggagtccccggggaccaagcaagagtctcaggaccgcatgggggacgtctcagtttctgtgtgtttgggtgggaaggttctgtatgggaatgcctgtgaggtggggtactagagagaagaaagttaatccgaagaactaagagggagaggggacagagacgggggttgtgtgtgtgtgtgtgtgtgtgtgtgtgtgtgtgtgtggcggcgggggtggggtgggggtgtctccgcaggacctgagttgcaggggtcccacgtgcgacagaaatccagataagagggcttacggaaggggcgccccctgggggcggaggggacaggaggtgtatgcaccaggcactcctagagatggggagccttgggggttcaggtgaggtgggggagggcgcaccacctgttgttccaggatgcaaaagtgggcagactctctgggatcgggccttcctgccccctaccccgcagaccctatggagtggggagtgggggcccctggagcggagaactcgcccagcctggcccccagccagcccagcccggccggcgtgagctcatctcccgcctgagcccccgcccgctgccactccctctctcctccccttcctccctctttctttcactctttcccggcccagccccaacgtgcctgtgccaggcgccatgcccaggcgggtgccaagcccttgggggtgcaaggagtggcatgtgaagtgggggctgacctcaaagggacccctatgtcctcaactccccgcttggccagggctgtggggcatgacgttggctgtggccatctagtggcccaccctgggtgtaggcacccccagggagctggcctgcgggcctgcattcaggccaagagaaggcagagaaatgcctctggagcctgggaaataccctcagacctggctggtttcccaccccctccatcagagacccttaccattctgcaattttgattcctagttaagcctctcctaggccccactactcaattaaaagcccccattagaccaccctggagtcatggccccacactcctacctaagggagctgagtttttgctccaggccctagacccgctctccaaccatggcaacagactttagagaccactcctccagtgaaattccctctcattcttccctcccacctcccaaactgtgcctttccccagataactctgtagtattgtccagaagtcctgttcttctagctcacccccttccccaggaacttctgatcccatttatcttcagagaaccccagggttggtcttgataagtgggttctcgcaagaggactccagacattgtgctccttacaggcagccattaataaaaagcctttggcacttaatccttagctgggaggggaggggattccacagagggactcggccaattgagcctcttaagtgctgatgacttccaggaattccacttcactatctaaaccctgttttcagcaccgtcaaactggttgtctcctaggcggcactagagtcctgttttctaggtaactgtccacaaaaggactcctgctgaaaggacgcctcagtttcctctgagctaggttcctccatcccagaaacctccagccgtctttgaactcgagaggactgccctttagtcgatctcctcccaggactccagtccctttatccttctaaacggaggtggctctctgcgtcctttcctctctacagttgtggctcagaatcccacacgcgcctcccccggccgcctgcctcctccctcccctccgcacagctgtctccagctgttctctgcagccccgggagagagagggagggggtgccctcgcaggacagcccggcctcctttggctcaaaggtcccggggctccccaagggtccggaatgtggagcctccgccctgcccccgccgcacctctctctttgtttaccccgccgcacagctggcagtgccccgcccagaggtggcctggggcccagcgagggcgcgcagaggggagtggaggcgcgttcccgctcgtggctccgccccggtgctactggaggctgtaaggccgagctagaactctaaagccccagccagaaagaagttgggcggagacacagcgcggtgggcggaactagggcacccggacagcctggaggtggggtgggaggggcgtgaacaagtgagccgggcgaatcagagtgcggaaaggaaggtgggaggggctagaggcagcaggttaagctcctggataggcggggtttagaatcccgcgggtagaaggtaatgtgagaggggctacaaagggggcacagccagggggcggaggtaggacttcgtggaccaagcagagggagtgtgggcgggcatagaatgcgaaggagacagagttgggcggggagttatgccagggagtggttaaaatgtggtttgagtcaatggaagggatcagcattgggtcggaggggctggaacagcaaagggttccgtcggactacggaatgggcggggtcagcgccgtctgggctggtctggaactccgtggactcagccagaaattgggatccgagaggttgacatgaaagtgaataaacagcctgagggctggctgggctcacgcctgtaatcccagcactttgggagactgaggcagaaggattgcctgagcccaggagttcaagaccagcctggccaacatagtgagaccccgcctctaccaaatttttaaaaaacgtagcccgatgtggtgcacgcctgtcgtctcagctacttaggaggctcagatgggaggatcacttcagcttaggaggtcgaggctgcagagccataatcacgctactgcattccagcctgggcaatagagcgagatcctgccgacttagtgggtgggaccgtacctctgatggctgaacaactggactgggccaatgctgatgccagaattggggcggggccacatgacagctaagggcggagcgacttagaaatgaatgataccttgagggagtagggaccccaggagaacctgccaggaatggtggcgcccctgagtgtcctcgttctcctcccagtcctgtgtcccttgatctgtcacaatggcggtgtgtgcgtgaagcctgaccgctgcctctgtcccccggacttcgctggcaagttctgccagttgcactcctcgggcgcccggcccccggccccggctgtaccaggcctcacccgctccgtgtacactatgccactggccaaccaccgcgacgacgagcacggtgaggaaagggtggccagagtcccctccgacccctgtcaagcatttcactttgcccctgaccctcatatttcccgccttcccgagcacctttgcccagctcgccctccccgccttgtctagccccaccccgtaagaacccgtgtagacatccgtttgcccggccgtgcctcccctaggcgtggcatctatggtgagcgtccacgtggagcacccgcaggaggcgtcggtggtggtgcaccaggtggagcgtgtgtctggcccttgggaggaggcggacgctgaggcggtggcgcgggcggaagcggcggcgcgggcggaggcggcagcgccctacacggtgttggcacagagcgcgccgcgggaggacggctactcagatgcctcgggcttcggttactgctttcgggagctgcgcggaggcgaagtgagaggaggcccgtggggaggggcccggagcttgcctccgcgcgggggcgcgctcacccaacacttccccgcagtgcgcgtccccgctgcccgggctccggacgcaggaggtctgctgccgaggggccggcttggcctggggcgttcacgactgtcagctgtgctccgagcgcctgggtaagccccaggacgtccccgaagtgctcggagctggggagtggtgacaacctcaccgttcctcctactctgccctagataaacccagttcacaaattgtagccacgcctaccccattgtggaggcgacttccagtcctgagcttttcataccgctctgggaccacccaccccattttctgactttggccactctgttctcaccctgcctttgtgccttggccccccaccatatactttgggagtctccaatgttgccatttcaactgccaacctaaggctgtccctgcacttaaactacagccaacatcttttagtccctcccaccccgtcttcgtctctgcccactccattctcgctctgcccacctgtccctggcccacccctctcctctcgccacagggaactccgaaagagtgagcgccccagatggaccttgtccaaccggctttgaaagagttaatgggtcctgcgaaggtgcaacggggcaggggtgggaggggcttggttctgggggcgggatttgcagggatcaagttcctgactccacggtgacctccccaaccctggcagatgtggatgagtgcgcgactggcgggcgctgccagcacggcgagtgtgcaaacacgcgcggcgggtacacgtgtgtgtgccccgacggctttctgctcgactcgtcccgcagcagctgcatctgtgagcaaccagcagggagctgaggctgggtcccgccctccctgccctcagaagtcccagagcatcctggggcctttaattccctcggacccccccagactcccgggttcctctgtcagccttagagccccctcagaacttctcagattcttatacagcttcagcatccctggaccacctttagacctttcaagccttttggatccagatccctccagactcccagacttttttttttggagatggagtctcgctctgtcgcccaggctggagtgcagtggcgcgatctcggctcactgcaatctctgcctcctggtttcaagtgattctcctgcctcggcctcccgagtagctagaactacaggcgggcaccaccatgcccggctaaatttttttgtatttttggtagagacagggtttcgccacgttggccaggctggcctcaaacccctggcttcaagtgatctgcccacctcagcctcccaaagtgctgggattacaggcgtaagccactgcgcccagctctcccagactttctggaatccttctcagacccccaaatcctctcgtccccctcaacgactcgactatcttcagatcttgagatccttctcagagtctctcattcaccactgacaccccattgaggctccaagcccttctcagacctccagagcccactgctctccttcagaccctctcagacctctcccaaatgcccctcgcacccaccaaccccccacccccaaccccagaaccattcccctctctcccaaatccctcagtgctacagcattcccagccccgccctgaaggatttcctccctgctcctcgcagcccaacacgtgatctcagaggccaaagggccctgcttccgcgtgctccgcgacggcggctgttcgctgcccattctgcggaacatcactaaacagatctgctgctgcagccgcgtaggcaaggcctggggccggggctgccagctctgcccacccttcggctcaggtgagcccctgcggcagtgcctagccctacgcgcaacacatgtggcgctcattctacgccccaccctccaaccctgagttcactgccccaacctgactggctgggctccagccttggccacgcagcagcctctgagacccgcaggcctcagactggacagcatccatggctccacctccatctgtagccacagccacccctccattcgtagccacagctaccccggagctcccaagttagtttttgtctcctgagcgtcaacgcccagtcccaccctgtccctgaactttgaccaagtccttggcttctgagacactgccttcagcttagccccaccccaggcatgcccctccctgagacacctccctggcattctatcttctgcttaaaactaaatctcacaagggagctatttcctagcctccaactctgtccctgtagacccaaccggaccctgccctcaaccctgttcctaccaccatcccaaatgtgggaaaacgctcttggccccacctgtagcctctaagacattctactctttggccaggacaccaccccatcccagcctccagctcaaacccctgacactctccagccaagccctgcccctagccaaggccagagtctgggctggccatcgttttcttcccgctctcttgtcctctctctgtctctcttacctattcccagagggtttccgggagatctgcccggctggtcctggttaccactactcggcctccgacctccgctacaacaccagacccctgggccaggagccaccccgagtgtcactcagccagcctcgtaccctgccagccacctctcggccatctgcaggtgagctggctctggcagaagtgggtgccatcttcaaggggctgccccagccccatagtgaaaggaggcaagagaggatttgggctccactcgttgataccccttctttatcaggctttctgcccacccatcgcctggagccccggcctgaaccccggcccgatccccggcccggccctgagcttcccttgcccagcatccctgcctggactggtcctgagattcctgaatcaggtttgctagaaagaactgagggcattgggcctgcagcagtggctcacgcctgtaatcccagcattttgggaggctgaggagggtggatcaccaggtcaggagttcgagaccagcctggccaacatggcgaaaccctgtctctactaaaactacaaaaattaactgggcgtggtggcagatgcctgtaatcccagctacttgggaggctgaggcaggagaatcacttgaacccaggaggcagaggttgcagtgagctgagatcgtgccattgcactccagcttgagcgacactccatctcaaaaaaaaaaaaaaaaaaagaattcagggcattgatgggggtattacaggtgggattctggggtaaaagactccaaggtgaagattccaggttacagcaaataggaatggtatgggcagagataagcgtttgaagggttgggtggtagttagatcaggagctggagtcacagttgggctcttctgtgtcaatcgagcaagccatttaacctctgagactcagttttctcattggtaaaatggggatggttgttgagatgcctctgaagtgtctgacacctagtaagtgctcagataaatacttatggtagataattatttttcattatctaatattagataattatttctcaggtagggcactaagaatgtatctcaggcggagggttggagagcaaactattcaaagccagagaccttggggtggcatgatgaggggattcggccaaggatctgatgagaacgttccaggataaaaaagatggagatcagaagaagactgggcttgcttggggaggagacatccatgaaggtgttttatggatgtctccgggggtgggggttttactgggatgaaaggaacggaggattcagagatgggggaaccctggctgactggaccccccaggtccctcctccggcatgtgtcagcgcaacccccaggtctgcggcccaggacgctgcatttcccggcccagcggctacacctgcgcttgcgactctggcttccggctcagcccccagggcacccgatgcattggtgagcaagacggagggcgcggaaggaggcggggcggggggctttgcctggtcaccttgtcaccagccccctccgtgtcctcagatgtggacgaatgtcgccgcgtgcccccgccctgtgctcccgggcgctgcgagaactcaccaggcagcttccgctgcgtgtgcggcccgggcttccgagccggcccacgggctgcggaatgcctgggtgagaaatttgccccacccggctccaggcccaccccagggtctcgctcctgctctcactccagagcctctccagccctcccacgcttcccctctcgggtcccgccccaggactgctctgccctggcccttggccctgcccttccctgacccgcctccacccagctccagcctccctttgaccccacccctacccagctcccaaactgccagtttctatcggggcctggtcgcaactcgtattgctccgcccccacgcccaggccccgcccttattggccacgcccctccctgactaactccctcccccaggccccgccactgtcctgcaactgcggcaaccaatctcctgaccgcgaccccgattccaaccctgccacaccagagccctatccccaaactgctcctttcttctctagcccctaccccaccttcccttagcttggtccccaaagctctcctttctgcccttgcgctaccaaaccctgccctccacaattgcctctctcactgtgcccctctccggctgtcctggccgggtccccatcctggctctggcccaagcttggtcccgctcccgcttccctctacccctgcctccttgcgtcgcttctcccggggctgtctgccccagtcccagccgcctggtctgtgcctacagatgtggacgagtgccaccgcgtgccgccgccgtgtgacctcgggcgctgcgagaacacgccaggcagcttcctgtgcgtgtgccccgccgggtaccaggctgcaccgcacggagccagctgccagggtgagggcctgggaggggcagctgggaaggggtgtgagcggttgggtagagcgcagtgatgagggccagagagactgaacaatagggcaaagcgagttgatttggagacagaggccaggctctcgagcagacgtgtggcctgatggcagtagagagagacctgggatgcagaggccaagtgatgggaaacagaaaggctgagtcatgaaagatggagaagcagaatgaggagggatggagatgtcagtaatcgggtaaggggagcagagttatggaggaaaggggagaaggaggccttctcatggggactacagagacagaaccagccaggcagcttagtagggattggtggaggatgcagagtcagatgatggtgacaaggaggaatagagatggggtcacggggacagaatgttggagggtggaaagccaaagtgacagaggtcagggaggcagaggggaacaagtgaccccgggccccctgccctgtgcagatgtggatgaatgcacccagagcccaggcctgtgtggccgaggggcctgcaagaacctgcctggctctttccgctgtgtttgcccggctggcttccggggctcggcgtgtgaagaggatgtggatgagtgtgcccaggagccgccgccctgtgggcccggccgctgtgacaacacggcaggctcctttcactgtgcctgccctgctggcttccgctcccgagggcccggggccccctgccaaggtgagggtgctgagcccagccctactccatcactgtttgctgtggagactggagagagattattgaggggcagagaggcagagtgatggggctcagggatggagaacaggggctgagggatggggacctcactccagagtcttctctcctttcaacaaaataaggcagtcctccccacccctcctccctttttgaaagatactcttgtgcttatgggaaccctgaggagggggtcacctgaggcagggcaggcaacatggagttggtgccttagcccccaccttagtagctggagagaccattgaatggggacacgaggaaggtgtctgtcttcctgggaagagggagcagcctgaggcaagtccagaaggcaggctcaagactggaatgctggggtgggtggtgatggccatgggaatggattcaggccccttcctcagcctcattggtcccctctgccccagatgtggatgagtgtgcccgaagccccccaccctgcacctacggccggtgtgagaacacagaaggcagcttccagtgtgtctgccccatgggcttccaacccaacactgctggctccgagtgcgagggtgaggccggggagggagggaggagtgtggatgggtgaggggggagttggaccacttcttcaaggccaccctctcccctgccccccagatgtggatgagtgtgagaaccacctcgcatgccctgggcaggagtgtgtgaactcgcccggctccttccagtgcaggacctgtccttctggccaccacctgcaccgtggcagatgcactggtgagaccaggccctggctgtgaccttgggcctgcatcatgacccccgacccttgacccagatcacaactatgacctggccgtaacctcctgacctggacctcagtccctcagtccccagagcctgtgactcctgaccctgacctggaccacaactcttgaccctgaatgtgactcctgagacccaacttgacagtaacatttgaccctaaccatcacccttgatgatagtccctgcccataccctgatagcaattgtgaccctcatgactctggccctgaatgtgacctataactcctgaatttgactgtgacatttgaccctaatcataaccactaaccatagcactgaccataaccttgactgtgtgactcttggtcctatctctttcacagcccctgcctcttctcagaatcccttctctggaacctggttgggttcataatccctgaccctggcccctgacttgaggcagaattctggaccctgactataacttctgatcctaactggattcagattccagtcactaactctgacccatggctcagccgaatcccatccaatgatcctagtttataatacttgactggtcccttgccctagttgatttctgatccctgtctgatccctctcctgactccaaaatcctggcacaggtagttcctagaaacctagacccacaggacttgcaaccatggtccccggaacccgagaaccttggtcagccctacccacctctgccatagagccctcccccagcctccaactcatgagacttcccaccacctcccccagacaccctactccaaggggattggtcgggtgtgtcccgagactggaccctttctgaacacccccaccccccacagatgtggacgaatgcagttcgggtgcccctccctgtggtccccacggccactgcactaacaccgaaggctccttccgctgcagctgcgcgccaggctaccgggcgccgtcgggtcggcccgggccctgcgcaggtgagcagcatagggacccgccagagagtctgggagtagggcctgggttccagggcaaagccggctggaaaggtggaggcgggaccaaggcgctgtgggaggagcttagaaacctggcattggtgggggcggggttactgcgatgtgggcggagcttgtctgggaggccgggtcccgtgactccgcccaatctcccgcgtaccctagacgtgaacgagtgcctggagggcgatttctgcttccctcacggcgagtgcctcaacactgacggctcctttgcctgtacttgtgcccctggctaccgacccggaccccgcggagcctcttgcctcggttcgtacccgggctgatcctggccccggaaagggtgggcttagggcaggaaaaggcgggacggggagaagagggcgaaaaggggaaaacgagtttttagccggggtattccagcaggatcagggggcagctggtgggagtctcgaggcagtgagggggggcggggcgtggagatgaaagggccgagtctgggtatttggaccgtgattgtaaagaagctgttctaaacccgtcggggggcggtgtttgcagggagggaagtagcgtgaggcaggttggggaaggcgtgagaggcctaggagagccgaggggcggtggaggggtgtggcctagaatgttaggcggagcgggaggtgggccgggccttcggacgccctgtcccgcagacgttgacgagtgcagcgaggaggacctttgccagagcggcatctgtaccaacaccgacggctccttcgagtgcatctgtcctccgggacaccgcgctggcccggacctcgcctcctgcctcggtgagaggccccgccccggcctgatccctcctcccttcgactccccgactcgccgattggcctcccacctctgtctttcctcctccgcttctcccctccccttacctctttcccccgcctcctctcctagcctccccaactctcctctaccccaatcttctcctgccctctcctctgcttccccggcctcccccttctgactctcctctcccctctttgtatcccccatcttgcctccctgcttcccacatccgaccacccgacctctctcctcagacgtggacgaatgtcgcgagcgaggcccagccctgtgcgggtcgcagcgctgtgagaactctcccggctcctaccgctgtgtccgggactgcgatcctgggtaccacgcgggccccgagggcacctgtgacggtgagcctgcccccacccgccttcgctagcgcttgcaacgcggtgctggaggctgctcccttggggactgaggaggggcgccctgcttcccagacttaggcggagggaaagaacaccctctcaccgtatctctgtagtggaaataagccgccaccgtgtccatgtattttcttgtgtcaggatcacggccaagcctcccttccagccgcagtccattccccctctgcagctgaagcccggtccacattttaggctatgcccccagtaagacaccgccccaaaactgttaggccccgccccttccaatcggcctctggattaaaaccccctcttcaggccccttctcttcctaacagaagtttggacgtgccctttccgcgccctccaccagtcctacttccagccttccctccaaagaggcccaatcccggtggccctaggccccattctttcccaatgagaaattcccaaggtgaaattctcctagcattggcccccgcccgtttaagctctgcccttccttggtctctctagacagggctttccattcgctcggatggctccagtctctcgaacacttaggccctcccttcctatctttgctccgcccacagacccgccccttgccttttcttactttccaggccttccctcccgccccgctctttcacccctcccgggtcaggctccagcgaggcaggagggtggggctaatcttccatacagtatatatttttttgtgtcggcgggggggggggggcggtgatggtgtgtgagtccgcatcttgtggaacacatgaaacaaaacatctgtgggctgccgttgactctgctagacctctgactcatggaaatctagcttcataagatctcattcattaattccacaagaattcactaagcacctactctatgccagcgtctggaaaaggtaataagagcaacaggaaataagtcatataaaaacccctgccttcctggagttattgtggggtaataaatatgtaaataaattcaagatttatacagcaggccaggagctgtggctcatgcctgtaatcccagcactttgggaggccaaggcggatggatcacgaggtcaggagatcgagactagcctggccaacatggtgaaaccccgtctctactaaaaatacaaaaattagctgggcatggtggcatgcgcctgtagtcccagctactcaggagactgaggcaggatactcgcttgaacccgggaggcggaggtggcagttgagccgagatcgcgccactgcactgcagcctcggcgacagagtgagattgtctcaaaaaaaaaatttatacaacaaaaaactggggggcaccttactatatgttgggcactggagatgtagcaaggaacaaaatagacacaaatccctgctattgtagagtttgtattctagagaggcgacagttggcataccaataaatacatacgtataacatcaggtttaggaaggaaaacaaagcaagacaaatgggacagagatgacagagcagtggatggaggagtgctaggagggcttgacctagacaccagacagaagcgagggaggcagccatgcagagagctgaggggagactgttcctggcagagggaagagccagtgcaaaggctctgaggcaggaccatgcttttgagaaatggcagtgaggacttgagagttatcaactatgctaggtgtggtggcacaagcttgtaatcccagctactcaggaggctaaggtgggagaactgcctgaacccgggaggttgaagctccagtgagccatgatcctgtcactacactccagcctgagcaacagagcaagaccccatttaaaaaaaagaaaaaaaagctggatggggtggctcatgcctataatcccagcactttggaaggctgaggcagctagattgcctgaggtcagaagttcatcaccagcctggccaatatgatgaaaccccatctctactaaagaaaaaaaaaaaagatagtggctgggcgtggtggctcacacctgtaatcctaggacttgaggaggccgaggtggacagatcgcttgaggtcaggagttcgagaccattctggtcaacatggtgaaatctcatctctactaaaaatacaaaaattagctaggcatgatggtgcatgcctgtagtcccagctgctctagaagctgaggtagaattgcttgaacctaggaggcaaaagttgcagtgagctgagattgagccactgcactccagcctgagtgaaagagcgagactccgtctcaaagaaaaaacaaagttttcaacattgttgatcttcagaagctcaggctcctagaataccagattgctaaagacttcttagtcttgggcaccggaagtggtgttagaacttctgaattcaggcctttgactcccctttcatctcctccacactctgcagatgtggatgagtgccaagaatatggtcccgagatttgtggagcccagcgttgtgagaacacccctggctcctaccgctgcacaccagcctgtgaccctggctatcagcccacgccagggggcggatgccagggtgggtgtccatcaggcatcgggtgagatgtggagatggtagaaggtccagaaatggcctgactgtctggtggttgcagatgtggacgaatgccggaaccggtccttctgcggtgcccacgccgtgtgccagaacctgcccggctccttccagtgcctctgtgaccaggttacgagggggcacgggatgggcgtcactgcgtgggtacgggacttcaggaggtggatgggaccaaagggggtgggggaggttggtcaggccctgtccctccccatatatctgaacaaatgggaattttcgggttgtggaatttagactttggaatataagatcatcttcatgccaggcacggtggctcacgcctgtaatcccagtactttgggaggccgaggcgggcagatcacttgaagtcaggagtttgaaaccagcctggtcaacatggtgaaaccccgtctctactaaaaatacaaaaattagccggatctggtggcaaatgcctgtaatcccagctactcaggaagctgaggcacaagaattgcctgaacccaagaggtggaggttgcagtgagccaagattgtgcaattgcactccagcctgggtgacagagtgagaccctgtctcaaaaaaaaaaaaaaaaaaaaaaaaatcagaactgttgtagtggctggtgacagaaacctgacttaatcaggttttatttttgtgtgtgttcttttaaaaatgtataaagtataaatgtataaagtactaatcttaagtgttcataaacattaatctttcctaacattttatgaggaaaatttctaaacacaaagcaaattgagagaatcatagaatgaacacctacctactcatgatctagattatattatcattttattacacttgcttcctcacatatctatccattaatccatctaattttttggacagagttaaaagtaaattgcaggcaccagtctgcttccccataaattcttcaatatgcatatcactaaccagaattcaatatgtgtttaccaatgtattcttctcttctctcttttttttttttactttgaggtggggtcttgctctgttgcccaggctggagtgtggttgtttgatcatagcttactgcagcctctaactcctgggctcaagccatcctccctccagaactgagcttccgagtagctgctaccacaggcacacgcaaccacgcccagctaatgtttttaatttttgtagagatgagttttttttttttttttggagacagagttttgctctagttgtccaggctggagtgcaatggcatgatcttggctcactgcaacctccacctcccgggttcaagcaattctcctgcctcagcctcccaagtagctgagattacaggtgcccaccactatgcccagctaattttttgtactttgagtagagatggagttttgccatgttggccaggctggtcttgaactcctgaccttaggtgatccgcccaccgtggcctcctaaagtgctgggattataagcatgagccaccacgcctggcctgtagagatgagttattgctctgttgcccagactggtctcaaactcctggctttaagcaaccctccctccttggcctcccaaagcactgggattacaggcatgagccattgtaccctacagatattttcattcaacgttcaatttatatacagtgaaatgcagcaatcttaattatacattctgtaaattttcacaaatgcaaacacaacaggttttatttctttaaagggaattaaaactataaagtcttgggatatatttgaactccaggcataactggatccaggaattttaacaaggctatttctatcttctctgagttctgctttcttctattgcatgattttcaggcagattatttttcacaatgatgaaggtggccctagaacctctgggctaacatctccagtgggccacccgagagctaaaaagggaggtttttttttcaatatttctagcaaaagcgccaggcctggctttgattgacccagcatgatcactgcctatgtctgaaccagagaaatgccgtcatctgtttggtcattcctggaacttgtgcccacctctcagagtagatatagccctactcaaagaacaggattgaaaatagaggagcacagggttcccaagggaaattcaaggtttctcactagaagaaaaacagatgatgggatctgggccattaaatatacacattatatttgaaagctgagactttcggacctttggaatcatagaagcttataaccactgagtccctctcccctgttgtctcctgcttacagatgtgaacgagtgtgaaacactacagggtgtatgtggagctgccctgtgtgaaaatgtcgaaggctccttcctctgtgtctgccccaacagcccggaagagtttgaccccatgactggacgctgtgttcccccacgaacttctgctggtgagactgatgtgtctatttaactgatggcggctggccccatgggaaaatcacgtggtctaatcattcctgatgtggacagctaccatcagctaaatactgttgttttaaacatcaacaaaagacacataagttaacatgggtagtgagactgtgtcaggagaattagaccctggtcctgactctaccagattctttcattcaacaaatgttcacagggtgtgcactgtgttcaggccatcttctgggcactgagaacatagtagtgaccagaagggatcactgtctgtcctcaaggggctcacaagccaatggggcagacagagatatcaccagatagtgacaacccagagaaatcaacaagactgtgacggggaaagcccaggggggttttggaggaggcgcctggcccagcatgagaagaggattagggagttcttcctgtaggagacgacatctgaactgagagctgtgggatgaatagaaatgagccaggcccgatgagtgaggagggcattctgggcagaggacacagcaagggcaaaggcctggcaaagccagagagcatctggcatgtgagaagaactgaaacaggttcaacatggcatgatttagagagcaaaggaaggggtagggagagatggggccagggaaagagtcaggtgcccgatcacactgacttatcacagtaagagagcactgcggtaccgtggaaggggtttcggcgttttttttttgaaaattttttttaaatgttttttttttaagacagggtcgtactttcacccaggccggagtgcagtggcgtgatctcagctcactgcaacctccacctcctgtgctcaaggaatcctccaacctcagcctgctgagtaactgggactacaggcacatatcaccacaacagctaatttttgtattttttgtagagatgtggttttgtcatgttgcccaggctgcagtatttttttttttttaatttttaaatgttacatatttaggctaggattggtggctgaaggccataatcccagctctctgggaggccaaggcgggaggatcacctgaggtcaggagttcgagaccagcatggccaacatggcaaaaccctgtctctactaaaaatacaaaaattagctgggtgtagtggtgcacgcctgtaatcccagctattcgaaaggctgaggcaggaaaatcgcttgaatccaggaggcggaggttgtgggattgcaccactgcactccagcctgggtgagagaggaagactccgtcccaaaaaaaaaaaaaaaaaaaaagctatgtatttatttatttttaataatttccatctttactttagattcaggggatacatgtgcaggctttttacctgggtatatttcgtgatgctgagctttgggatacaaatgatcccgtcacccagatagtaagcacagtacccaacagccagtttttttttttctttgagacggagttttgctcttgttgcccaggctagagtgcagtggcgtgatctcagctcactgtaacctccacctccctggttcaagtgattctcctgcctcagcctcctgagtagctgggattacaggtgtccgccaccacgcccagctaattttttgtatttttagtacagacggggtttcatcatattggtcaggctggtctcgaactcctgacctcaggtcatccacctgcctcggcttcccaaagtgcagggattacaggcgtgagccaccatgctcggcccaacagccagtttttcaacccttgtacctctgtccctcccccatctagtggtccccagcaactattgtgggcatctttacatccatgagtacccactgtttagctctcacttgtaagtgagaacatgtggtatttggtcttctgtttctgcattaatttgcttaggataatggcctccagctgcatccatgttgctgctaaggacatgatttcattctttttcatggctgtgtagtattcatggaagggttctaagcaggaaagatagaatcagattttgaaacctccctctggccgccatgtgaaggtgattgaagcgggtgatatggaggctgggagctcagggaacaggctggggcaggggcctggacaggagagaacagggctggaccaaggcaggggctgtggaaggggaggagaaggtggtgggagaaaaacccaagaggccatgtggacaagctgtggatgtctgggtggggaagctgtgtccaagacaaggcataggttttttgtttgtttgttttgtgacagagtctcgctctgtcgcccaggctagaatgcagtggtgtgatctcggctcactgcaacttccgctcccgggttcaagcgattctcctgcctcagcctcccaagtagctgggattacaggcatgcgccaccacacccggctaattttttctatttttagtagagaccgggtttcaccatgttggtcaggctggtctcgaactcctgacctcaagtaatccaaccgcgtcggcctcctgaagtgctgggaggacaggcaggagccactgcacctggcgacaaattgtaggttttgagatgaagactcagccagtttgccacacactggataagagacccagagaggcatccaggaagccagggaagttccctcaccacccacccaagctgttagctgcagtgacgggaaagtgtgaggtggggaggcaagagatgcagaaatgacaggaggtgacagtgggagaaagagaaacagtgacagaggagcgtgagaggtgtggagtctggttctgccactgatggctgccaccctctgtttccctatctatgccagcctcagtttccccatctatgatagtggcggggctggggattcagcccacactgggctaaagctccttgtctccccaggcacgttcccaggctcgcagccccaggcacctgctagccccgttctgcccgccaggccacctccgccacccctgccccgccgacccagcacacctaggcagggccctgtggggagtgggcgccgggagtgctactttgacacagcggccccggatgcatgtgacaacatcctggctcggaatgtgacatggcaggagtgctgctgtactgtgggtgagggctggggcagcggctgccgcatccagcagtgcccgggcaccgagacaggtgggcatgggctgatggggacacagggctgagggcttgggtggaaatactgggtggggtgtgggcctgggacaggggacacttttggacagggccttgaggtactagtactgtcagggcaagggcgagctgccaggcaggtgggcatgggcagacataaggctgagaggtgggcactaacaggcacagggccagagggacttttgtgacaagtgggcacgagcaggtcagggctggggctggggctctggtgtcctggctcaggcttgtctctgtgtgtagctgagtaccagtcattgtgccctcacggccggggctacctggcgcccagtggagacctgagcctccggagaggtgaggccagcctttgaccctccaccccactcagctctgaggcccagcttcgtctcttcctgttttctttgcctctgtctctcaccctttctgtttctctgtatctgtctccccgaccccacccatactctgtctctttctttcttttctttcttttttttttttttcttaagatagagttttgttcttgtcgcccaggctggagagcaatggcgagatcttggctcactgcaacctttgtctcctgggttcaagcgattctcttgcctcagcctctggcgtagctggctacagacactagccactacgcctggctaatttttttgtatttttagtagagacagggtttcaccatgttggccagattggtctcgaactcctgacctcaagtgatccgcccaccttagcctcctgaagtgctgggatttcaggcatgagccaccgctcctggcctctccatctttctttccctgtctctacttcttggtctatcaccagctctgtctctgtcttcccacgtcatgccctcacacactccacccatccagcccgcccccatctctctctctgcttttcgcacagacgtggacgaatgtcagctcttccgagaccaggtgtgcaagagtggcgtgtgtgtgaacacggccccgggctactcatgctattgcagcaacggctactactaccacacacagcggctggagtgcatcggtacaagccccacctcccccaacccccggcaactctctccaacccctagccttgccagctcccctctggaatgtggccaccaccagcgggaagtctttcctggagtctagactccatccatcacactgccaatgtgctgggaagagaaatgggaaagggtggggagagttgaaggggatgcctcttaatcatcctctccctagacaatgacgagtgcgccgatgaggaaccggcctgtgagggcggccgctgtgtcaacactgtgggctcttatcactgtacctgcgagcccccactggtgctggatggctcgcagcgccgctgcgtctccaacgagagccagagcctcggtaaccccgcccacgccatccaggccctccttcccttggctcggcctcacacccgcacccgggccacacctttgcgacggccacgccctccgaaggccacgccccatgctcctggctcgaccacgccccactgagccctcactcagtctctgtcccactgtctgtctctctgaggcgagctggctaggtggttaaaggcaaaagttttagggcaacctttgcatgttgcctcttggactccaggtttctttctgcacactgggtgaatagcccctaccttatagggtggctgtgaggaattttatttaattttatttatttattttttgagacggagtttcgctcttgttgcccaggctagagcgcagtggcgccatctcagctcactgcaacctccacctccggggttcaaacgattctcctgcctcaggctcccgagtagctgggattacaggcatgtgctaccacgcccagctaattttgtatttttttctttagtagagacagggtttctccatgttggtcaggctggtctcgaactcccgacctcaggtgatccgcctgccttggcctcccaaagtgctaggattacaggtgtgagccaccacacctggctgatgaattttattttattttattttatttttaatttttaatttttagttttattatttattttttactaagtaccacatgagtgctgcctgttattatttatctgcatctctaggtctctgtctctttccagctgtttgtggatttctgtctttggggttttctctgaatttctgacaccctctggatctttgttttcaggtccctttctcctgcctctgtatctttgttttcctgttttgagtctttgactctttggatgtgtttctctctctctctttgggtcttttttctcccttttcgtttctgactattttcctatctctaggcccctaacaacttctgctgaagctctgtctcttgaacactctacttgtcgttgtttgttttcatttttgagacaagatctcgctctgtcatccaagctgaagtacagtggcacaatcacagctcactgcagcttccacctctcatgcccaaatgatcctcccacctcagacttgcagtagctgggagtccaggagcccgccaccaagcctggctaatttttgtatttatatatatatatatatatatatatatatatatatatatatatatatatatatatatatatatatttttttttttaaagatgggtttttgccatgttgcccaggctggtcttgaactcatgggctcaagcaacccccccacctcagcctcccaaagtgctgggattacaggctccaacacactctctctgtgcttcttgccacctctctttttgcccactgtcccaaccactcttctctcatttcctgtgccttcagcacttacccccatacagtgagatgtcagacaatgctcagagaagcaaagcaactgccccgaggccacacagccctccagaggccagacttgggtagacccctctcaggccctacctcccagtcattggacaccttcttctgccctctcctcctatatgacagtctccttgttcctcaatccagaaccctggagtgacctcagagaacttccagagttagatcacagctgatgagaaggatttgctgataggcagagggtgcctgggctgctcagcaggggaagggtccagcccctgggaggacctgagtgccaggcccactctgacacatgctgtctccacctacagatgacaatctgggagtgtgctggcaggaagtgggggctgacctcgtgtgcagccaccctcggctggaccgtcaggccacctacacagagtgctgctgcctgtatggagaggcctggggcatggactgcgccctctgccctgcgcaggactcaggtgctggcactggcctaggctgaactcagaggccttgccccatgggctccaaatctaggccctcagatccccagtcgcagaacccccagactctatccaaactctgtcccctagaccaacccctatgtcgcagcccatcccaaaatcttggtccccgattcacactccaaagacttccaagtctcagccacagacatcccaaggcctgacccagcagatcccatagtaccagacccacaggcccctgaggtctggtctctcatatgctcagctcctggtctctagaaccaccagacacagcctatgagatgtcctcccctaaatcctgggcccttagacctgaaatgccagttgctcagacctcaccatcctggccccagaccccctggtcacagccccacagacctccaagtcccattgccttagacaccctccaagtcccattgccttacacaccacatccgggtgcctaagaccccaacacacagccccagagattcccaagtcttggtttctacaacctctgagtccctcagtcctcagcccttaggaacccagttccaaagccccagtcctgggtcctcagacctccaggtcccagtgcctcagactgtctcccatgcatcctgccccctcaggtccccagcagcaatccctgggattccccagtcctggtccacagaacacccccatctaatttcttcaggtccctagtcctagctcccatgggtcgctccaattccagcctctcagaccctgattcttagcaggcttcattacccttgatctcagcacgtccgaatcccagtctcagccttcagattctcagcgctggctccagaaaccccggggccacccaggaccctccccatccagtcctctgcctcctctcccaaggggggtatgtgagtggtgggtgtgggggccagcaggggctgattgtttgccttggctcctgttcccagatgacttcgaggccctgtgcaatgtgctacgcccccccgcatatagccccccgcgaccaggtggctttggactcccctacgagtacggcccagacttaggtccaccttaccagggcctcccatatgggcctgagttgtacccaccacctgcgctaccctacgacccctacccaccgccacctgggcccttcgcccgccgggaggctccttatggggcaccccgcttcgacatgccagactttgaggacgatggtggcccctatggcgaatctgaggctcctgcgccacctggcccgggcacccgctggccctatcggtcccgggacacccgccgctccttcccagagcccgaggagcctcctgaaggtggaagctatgctggtgagcactgccagcgcatgatgagactgagatgaggggtgaggtgtgcacggagagaggagggagggaaacagaaggcgggaggagagacggaacacaggtgcaactggagagagccacagaaagggacagagaagtgtctatagcccggcaaagaaagagaaggtggcagagggaaagctggcgagagaatgaggtaagcttactggccccgcagcacccgccacagccctggagcgggatggacagtttacagcgagaaagatggagccaaggacgcgcacccaccgttgtgggtaaggggtgaaggcagggacctcaagtcatagggtccccgcatttcccacaggttccctggctgagccctacgaggagctggaggcggaggagtgcgggatcctggacggctgcaccaacggccgctgcgtgcgcgtccccgaaggcttcacctgccgttgcttcgacggctaccgcctggacatgacccgcatggcctgcgttggtgagggcgggcccggggccagcatgcgcagggagaggcgaggcttgtccagggagggtggaagccctgactagggggtgctggtcagggacgggaaaggacctcactacaggggacgggccagcgcaaaagggaggagtaattcttccacctgggtggggcttgactgaagaaggcggagtcagggtgccagcagggcagggcttaaccacggggctggagccgggccttgaagcaggatagaggaatagaatagtgctggtgtgacttgcagtgctaccttgggcagtctgcaggacctctttggagagcctcagtcttcccacctgtaaaatgggaatattaatcaagtctgcctaatttggatggcagtaaagccaagtgattaaaatcaagaacatagccgggcgcagtggctcacgcctgtaatcccagcactttgggaggccgaggcgggcggatcattaggtcaggagtttgagaccagcctggccaatatggtgaaaccccgtctctaaaaatacaaaattagccgggtgtggtggtgcatgcgtgtaatcccagctactcgggaggaacccaggaggcagaggttgtggtgagccaagatcgcgccattgcactccagcctgggcaataagagcaaaactccgtctcaaaaaacaaacaaaactcgaggaggtcacatagacctcagactcagtccagcaacttgctgtgtgacgctggacaggtcactgtcccttacctaatttttctgatatgaaaaatggctacagtcaaagatcccatttcatagaattgcgaatattattaacactgatttacaataacattgagagtcatgtgcaaatggaggcacatgcgtaaaacgaattacagtacctacctaataggggtattagaaggacaaagttaatacttgtaatgtgcttaaaacaatgcatgaacatttattgttgttgacaccatcatcagtatttttttcttcttttttttttcttttcttggcgacagagactcactctgtcgcccaggctggagtgcagtggcctgatctcggctcacagcaacctcctcctcccaggttcaagcgattctcctgcctcagcctctcgagtagctgggattacaggcgcccgccaccatgcctggctaatttttgtattcttagtagagacggggtctcaccatgttggccaggttcgtctcgaactccggcctcaagtgatccacccgcctcagcctcccaaagtgctgagattacaggcatgagccaccgcgcccggccattattattttcttaacaaaatcctagtgcggtatgggccaccatatccatcttacagaacacgaaactgaagcacagtgaggttaagccacctggccgggctcacacagttagcagatggcagaggccagattggactccaaactgctcagcatctgtgctcctctgttccaagaacttaaggggccaaggaggcgagcttctggggccccagccttcagcagcgatcgttgtctcccctccgcagacatcaacgagtgtgatgaggccgaggctgcctccccgctgtgcgtcaacgcgcgttgcctcaacacggatggctccttccgctgcatctgccgcccgggattcgcacccacgcaccagccgcaccactgtgcgcccgcacggccccgggcctgagccctggcacccgctggccgcccacccgcgcccgccactcggggcccctgccgcgcatcctgcagcccgcttatgcgtatgtgcacggggccgcccgcctggacctggagaagggacctacggacgcctggaagctgcgacgccctgcactgctcccgcctccaccagcgcctcccactgatgtcgtggtcccgggcctggcccaggggcccctttacatgccctctcccttttataaaattttccattaaaaaccacctattttctatcttttgcctcctcctgtctatttctccaagtctaccccagtttgtgctttgagtcactaaatgacattcccttcccttctgacccctagtttcgtctgtctctctgcctctcttggtctctgtcttccttctctctctgtctctgtcgttctatctcatctctgtctctttctgatttgttttctctgcctaacctctacatcctgtcttttcatttctgtttctgcatgcgtctttctcttttcccttctctttgtttggttctttcatctcgagagcaatcttgcctctggattttttttttttttttttttttttttttgagatggagtctcactctgtcacccaggctggagtgcactggcgccatctccatccactgcaacctctgcctcccggtttcaagcgattctcctgccttagcctcccaaggagccgggattacaggtgcacactacaacccaggctaatttttgtatttttagtagagacggagtttcgccatgttggccaggctggtctcaaactcctggcttcaggtgatccacccaccttggcctcccaaagtgctgggattacaggtgtgagccaccgcacccagcttttattttttagagatgaggtctccctatgttgcccagtctggtctcaaattcctgggctcaagtgatcctcccacctcggcctcccaaagtgctgagattatagatgtgagccaccgtgcttggcctagaagtcttaagctgtcctggaacaagtggggcaagagcagggagcaggagtccctggagggccaaacccagacctgacgctccagctaggagttggctgtggatggagtatggagcggcagtcctgacagctggaggcgggaatgagctctgtgtgtgatggtggagtgcccagcacggacctcctccatgtgccctgcctgcttactggccatgttcctagtccagactcctcctccctaaaaaacaagaacggcctagatcagcataaacttagggcaggagtaggggtcgggaatcgtgggtgatcaacttggcactgggccaagctgggaaaaaaaaaaaaaatgtacctcagccaggcgcgatggctcacacatgtaatcccagcgctttgggaggcccaggcaggcggatcacgaggtcaagagatcaagaccatcctggccaacatggtgaaacctggtctcaactaaaaatataaaaattagcccgccatggtggcatgcaactgtaatcccagctatttaggaggcggagataggagaatcacttaaacccaggaggcagaggttgcagtgagccgagatgacgccactgcactccagtctggcgacagagccagactgcatctcaaaaacaaaaacccacaaaactgtacctgtcattcttcacccacctgtcaattcttgtgtccccagggtttcaaactcatatgctgcaaagaccagaaccggaatgttagcaggaagcagggaaaattgaggcacacattaccttctgcctaaggcttggttagactaggaaggggaattttccccattgttaatacttttttttggcctggcggggtggctcacgattgtaatcccagcacttcgggaggccaaggcaggatgatcacttgagctcgggagtttgagaccagcctggcaacatagtgagaccccatctctacaaaaaagtagctgggtgcgatggtacatgcctgtagtcccaactacttgggaggctgaggtgggatgattgcttgagcccaggaggtcgagactgcagtgagctgtgatcgtgcctctgcattccagcctgggtgacaaaggcataccttgtctccaaaaaagttttttttaatgtatatttttaataactatctgatattgtttggctgtgtccccacccaaatctcatctcaaattgtaatcagaattgtaatccggggaaggacctggtgggaggtgattggatcatggggacagtttcccccatgctgttatcgtgatagtgagtgagttctcacaagatctgatggttttataaggggctcttctcccttggctcgctgtctctcctgccgccttgtgaagaaggtgactgcttcccctttgccttctgccatgattgtaagtttctgaggcctccctagccatgcagaactgtgaatcaagtaaacctctttcctttataaattacccagtcttgggtggtatctttatggcagagtaagaatggaccaatagggccgggcacggtggctcatgcctgcaatcccagcactttgggaggctgaaacaggtggatcacctgaggtctggagttcaagagcagcctggccaacatggtgaaaccccatctctactaaaaatacaaaaattagcagagcatggtggtggatgcctgtaatactacctactcgggaggctgaggcaggagaatcgcttgaacccaggagccggaggttgcagtgagctgagattgggccactgcactccagcctgggtgacagagcaagactctgtctcaaaaaaaccaaaaacaaacagaaaaagactaatatagtaccctatggggcctatttggcctatgaggctccagttttaatcctctaggcactgctgaaattgcataaattgtggattggaggctgcttcctgacctgatctccagcccacccgctgagtgtcaggaggggtcacatgcccagcagcctcctgtgctacatcggggaatcatactaagagcgcctaccctgcaggattggtggtggtggttaaatgaattgtcatatatgtgcttagaatgtcacctggtacagagcaagggctcagtcattgttggcataaccctgggcctcccaggatatgctgagccctgtggctgggcagatagtaaggtcttgttcttttctcttttcctttccttcttccttttcttttctttctttctttctttcttttttttttttgacatagggtcttgctctgttgcccaggttggagtgcagtggtgccaccatggctcactgcagcctcaacctcccaggctcaagtgatcctcccacctcagcctctcaattagctggaactgcaggcatgtgccaccatgctcagctaattttttaatttttattttttgtagagaggggaccttactgtgttgcacaagctgatctcaaactcctgggctaaagtgattctcctgcactttggcctgccaaagtgctgggattacatgtatgacccacggcacctggcctggctaggtctttttctttaccaattctccctcccagggtggaggaggaggccagaccccatccccttcaacaggacagtgagaaacacctaacacaaggccttggtggggaggagtgtcagacaaggagtggctcaaggctccacaaaggggtgacagttgcacaaaactgtctgtaccaaaaatcattgaattatacactacacatgggtgacttgtgtggtatgtgaatcataagtcaataaagcccttctttaaaaacgccgactctacaaagcgtttcagaaagttaccctccagctgatacagggaacccgacccaaccactaattcctcagtcattcattcaacaaatattttcctgctttgcaataagtgaattcgaggaacacaaactttaacccagccttggggggtggttagggagggctcctggaggaggagatgttggagctgagagttgattactgtagggaaatgagcaagatgaaaggggtggaggtgcctataatcccagctactccggaggctgaggcaggagaatctcttgaacccgggaggcagaggttgcagtgagccaggattgcaccgctgcactccagcctgggcaacagagtgagactctgtcagaaagaaagagagagagagagaggaaggaaggaaggaaagaaggaaggaaaagaaagaaagaaagaaagaaagaaagaaagaaagaaagaaagaaagaaagaaagagaaagaaagaaagaaagagagagaaaaagaaaagagaaagaggtggagaaccatgttccaggtggaggggacagcacttccgaaggcctagaagtaagagagcaagtgtctgttagatgtctttcctttggtatctaaggcctgtattggacaacaatggtgacaatcttggtgcctgagaaatcccaggcccttgccttcatgggggacaccgacacatcaccacacatgagagcctgtgctgtatgggggaggcacaggcaggggtcaggctgcggtgggggaagatcagagggcatgcatgcccagaggagacacttgatccagtctgagagatgatcagggaaggctttctggagaaagagacaaagctctgaacagtctctgtttgggcgtctagatccctttgagaatctgtcgaaagctcattcacagaagaaaaactcacacaaacaaaggttgcaggcaatttccaagtattctcatccttcctcggaatcttctgtgggtatcccattcccaccccgcccccacaaccaccttgctgcataaatgtcccaggaattgcgacagggctggccaacagtgctggaaaactgcaatcgaatttcagcatcctgggagtgggatctttcttctgagatctcgcccgccgccgggcctggagggtgtgccccatggatctaattgttatcctgcctgcatcccctgcactaacccccagacctggcgttggggcttctggcactggccgggaccgacgcgttctcctctggaatgttcagagggaggtagtccaggcggtcaccgcgctcgccagacaagcaacgcagcgccccccgatggccaccgcgcgccggcgcagcccaagccggctgcgccccctagtggccgatgctcgtaaagtcttcagcgagccccgagcttttgctagccagcccttggttttgcaggctggggtcatcgtcgttgtcatcgttggcatcatcatcatcatcatcatcatcatcatcatcatcaatagctagtaggcttggcgtggtggcccacacctgtaatcctagcactttgggaggccgaggcaggtggatcacttgaggccagaagttcgagaccagcctggccaacatggtgaaaccccgtctctacaaaacagtacaaaaaagcaaccgggtgtggtggtgggcacctgtaatcccagctactcgggaggctgaggcatgagaatagcttgaacctggaaggcggaggttacagtgaactgagatcgaaccactgcactccagcctgggcaacagagtgagactctgtctcaaaaaaaaaaaaaaaaaaaaagctatgatcatccaggcagtgattcatgcctgtaaccgcagggctttgggaggccaaggcaggaggatgtcatgaggccatgagttctagactaacctgggcgacagagcaagagctgtctctataaaacaaaacaaaatgctatagaccagacgcagtagctcacacctataatcccagtgctttgggaggccaaggcaggcggatcacctgaggtcaggagttcgagaccagcctggccaacatggtgaaaccccatctctacactttgaaaaccactactttaactgtctctcccacagggagagctgcctccttcactctcagctcatttggttcacatgttgaccctactggaagactccagacctggccagtgagagaatcccttcccttcagcctttgcaactggcttagggacaggtgtgtccctgaagctggactgatgagcattggctctggaactactgctgattgttgaagaaggggcttcctagacatactgtgctgaaagggcacaggcttggaacactgggttcttacatgggagaagagctggcctgaaaatgcagccaacacgaggggaagcagcgctgggagaggaacagagagactggtgcctggcgcacgtcatctgcacctctgtctcacccttctccagaacatttctttgcatcttacatcaaatttgtcagcaaattctgtcggctctagttagaaaaggtgtcccattctgtgttggaggaaaagcgatgctgtctatcaggacgttaccgggtcagttgacagaactggaatacgaacagtcgattagaaaaattcacatactaatattaaatttactgaagttgatcactatattgtggtcatgtaagagaatatccctattcttttgttttgctttgttctgtttttgagacggagtctcgctctgttgcccaggctggagtgcagtggtgcaatctcagctcctgcaacctcctcctcctgggttcaagcaattctcctgcctcagcctcccgagtagctgggactacaggtgtgtgccaccatgcccagctaattttcgtattaatatttccagtacagacggggtttcaccatgttggccagggttctcgaacttacggcctcaagtgatctgcccacctttgcttcccaaagtgctgggaccacaggtgtaagccacgtcgcctggccagaaatatccctattcttttcttttcttttcttttttgagatggagttttgctcttgttgcccaggctggagtgcaatggcgaaatctcggctcactgaagcctctgcctctggggttcgagaaattctcctgccttagcctcctgagtagctgggattacaggcacccaccaccacacccagctaattttttgtatttttagtagagacagagttttgccatgttagccaggctggtcttgaacttctgacctcaggtgatccgcccccctcggcctcccaaagtgttgggattacaggcatgagccaccgctcccggccaaatatcccaattcttaagaaatacatagggcggctgagcacattacctcatgcttgtaatctcagcactttgggacactgaggtgggacgatcccttgagcccaagagttcaagaccaacctgggcaacatggcaagaacccatctctcaaaaactttaaaaatgatccaggtgtggtggctgcagtgtcaaggctgcagtgagccaggatcatgccactgcactccagcctgggtgacagagtgagaccctgtctctacggaaaaaaagaaagaaaagaaaaagagatacattacagaagtttttgtttgtttgtttgtttgtttgtttgagacagtcttgctctgtcgcccaggctggagtgcagagtgcagttcagttcactgaaatcctggttcactgcaacctcacctcccgggttcaagtgattctcctgcctcagcctcctgagtagccaggactacaggtgtgtgccaccacacccaggtaatttttgtatttttagtagagacaaggtttcttcatcttggctgggctggtctcgaacttctgacctcagatgattcgcctgccttggccttccgaagttctgagattataggcatgagccaccgtgcccagcccattatagaagcttttaggggtgatgttgtttgtaacttagcctcaaagatttttttaaaaatcagaaaaagaaaatacataacagagaagagagcaagtgataaaggaaatgaggtagaaatgttaccaatccatgaacctgagtataaaatacaagagagttctttgtcctattatttgtcttgcaactttttggtaaatttgaaattatttccaaaggaaaagttaaaacactttctaaaattgagagatctaaaatcttaggccaggcgtggtggctcacacctgtaatcccagcactttgggaggctgaggcaggtggatcacatgaggtcaggagttggagaccagcccggccaatatggtgaaaacctgtctctgctaaaaatacaaaaatcagctgggcatggtgggaggtgcttgtaatctcagctactcaggcacgagaatcgcttgaacctggggagcggaggttgcagtgagccgagatggcgccactgcactccaccctgggtaacagagcaagactccaactcaaaaataatatgaaataaaataaaatcttagacaacactatcattataagtaggattggggagatcaaaaacaacatgctctgtgctccaggtactgtttggtgtgatgttgaaaatattagattttaaacttgattttttcttttttttttttttgaggcagagtgttgctctgtcgcccaggctggagtgcagtggcgtcatctcggctcactgtaacctccgcctcctaggttcaagcgactcttctgcctcagcctcctgagtagctgggactacaggtgtgcgccaccacacctggctaatttttgtatttttagtagagacggggtttcaccatattggccaggctgatctcgaactcctgacctcatgatctgccctcctcggcctcccaaagtgctgagataacaggcgtgagccactgcgcctagcctagacttgattttttaaatttctgtcctccaatagaaatagacctgatttttaaaaatgtgcatggccaggcacagtaattcatgcctataatcctaggactttgggaggccaatgtgggaggatcacttgagcccaggagttggagatcaacctggacaacatagctagaccctgtctctacaaaaataaaaatattaggtgggtgtggtcttgtacacctgtagtgccagctacacagggggttgaggagggaggatccattgagaccaggagttccagcctgcaacgagctaggatctcaccactgcactgcaggctgggcaacagagcaagaccctgcctcaaaaaaaaaaaaaaaaaaaaaagcatgttaaaatttcaaagattgctacaaaaagaaagaaatggtgcatagattccaaactagtagagggaaaacaaagaataagaaaacataagacaaaactaaacaaaaaatgcagccaattcaaagaagcttaaatgagcatgaaaaaataaataaacgtagaaaaatgaggacactagaatcacaaagcaaaatggtagaagtaaataaaatacatcattaatcacatacacaaaaaatgtattcagggccaggtatggtggctcacacctgtaatcccagcactttgtgaggctggggcagacggatcacttgaggcaacaagtttgagactagcctggccaacatggtgaaaccccatctctaccaaaaatacaaaaaaaaaaaaaaaaaaaaagaagaagaagttatccaggcattgtggtgcacgcctgtaatcccagctacttgggaggctgaggtaggagaattgcttgaatccaggaagtggaggttgcagtgagctcagattgcaccactgcactccagcctgggtgacagagtgagactccctctcaaaaacaaacaaagaaaaaatgtattcagaggccaaatacttcttaccacctccatagctgccacccatatcttaccctcctcctagggtttggagttcctactcctggccccacaatctctccccgtcatgcagatagagggactctgggaataaccaagtcaactcaactccctctgctgctcaaaactttcctgtggctccatctcactcaaagattattttgctcattttttttttgtttttttttgagacagggtctcactctgttgcccaggctggagtgcagtggtgcaagtcatgactcactgcagcctagacttcctcggctcaagccatcctccaggcctccagggctaccacaacccagccaattttttgatagagacgaggcctcccttattgcccaagctggtcttgaacttatgggctcaacttatctacccacctcggcctcccaaaatgctgggattccaggcctgagccactgtgttccgccaatctcattcaaagtaagagccaaagtcctcactgtggtccagcagactctgcaccgtctgcttcttgttccctctctgacctcacctcccctccaccccccacacgctcctctgcagcaacacggatctccttgttgtttctctaagatccagacatgttcccatcccaccccaggaccttggcactggctgttccctctgcctaggggactctttccactaggttcagtttctctttttttttttttttgagacggagttttttcactcttgttgcccagactggagtgcagtggcacgatctcggctcacagcaagctcctcctctcaggttcaggcaattctcctgcctcagcctcccgagtagctgggactacaggcacccgccaccacccccagctaatttttgtattattagtagagacggggtttcaccatgtggccaggctggtctcgaactgctgatctcaagtgatccatctacctcagtctcccaaagtgctgggattacaggtgtgagccaccgcgcctggcccccttggctcagtccctcaccaccagcagaagaatggataaactgatcccacagtggcctgcaatgtgtgtgtgctgcaatgagaataacctgtaactgcacacgacaaccaggatgaatttttttcacggtgctgactgaaaaaagccagatgctaaaaagaacatattttcagattgcatttacatgaattcaaaacaggcacaaccaatctatgctgtcagaagtgagggtgataatgaccttggagggagggcaggtgataagggagcgccgagggggcttctggggggctggagatattgcttcttgggctggtggctggtgacacaggtgtgttcaatcggtgataattcctccagcggttacacttacggtttgtactttcctgaatatgggtctacttcaagaaaccactgctgaaaacgaggagctgcacccaccacccagtgtcccctccatccagtttaagcaacaggacattctcagccccccggggtccctccctcccaatgcaatgtgcttttatgtcttcaaggggcctggccaccaccttggttgcttcccccagcttgctatgcatgtcaccccacgcatgttccaggcagctccacagaggtccccagagagaaggcaggctgtgggtgggaggaagggcaggggtgggggacgtggcttcccagcggagcccgggcagggggaggaaacatttctgcaacaccccacaccaagtcctgacttcgggggggctttgcagggtgaattctaagtgcccagggccccatgtgaaggggtcaactgtgtctcactctgtgtctgtctctctgtctgtctctcttcctgtttcctccctccctcccagtctctctctcctcaagaagaaaaaaactcacaactatttacataacaattttatatcagactgtaacccaaaaacccaagttccaacttcgaatttttcaataaactttgaataaatagtagtgctatttttctttctttttttttttagtactgtaatgtttacttttaaatttaatttttactggcagactttacgtatatgaggttcaagaaaagaaaatctccactgtttacatttcttttctgactattaatattgttcatttcatttcctgattattactggcaataattttgtcctatggggaagggaggcgcttggcaggcctcgagttatcctttgagacatgggggtctgtggtcagatttagaaagtgcttgtgggtggctgggtgggagtgggtcatgggagacataagggcaggctggagtccagggaggaggccggccgggcagggacccaggcagcggagatggacagggccatggagaatctgggaggcgcaggccctggggactgtggattggggaaggagataattttattttattatatttttttgagacagagtttcagtctgtctccaagactgtagtgcaatggcacgatctctgctcactgcaacctctgcctcccgagttcaagggattttcctgcctcagcctcccaagtagctgggattacaggcacgcaccctcacacccagctaatttttaatatttttggtagagacggggtttcaccatattggccaggctggtctcatactcctgacctcaagcgatccacctgccccggcctcccaaagtgctgggattacaggcatgagccacgacgtctggcctaattttttgtatttttaggagagacagggttttgccatgttggccaggctagtcttgaactcctggcctcaagtgatccgcacacctcaacctcccaaagtgctgggattacaggtgtgagccactacgccccgccaagggaaggggagaatttgagtgctattgaaggtagaaaacacagaacataggctgggtgtggtggctcatatctctaatcccagcactttgggaggccaaagtgggaggctggcttaatcccaggagttcgagatcagcctgggcaacatagtgagacccctgtctctacaaaaaattaaaaattagccaggcatggtggcacccacctgtactcccaaggctacttaggaggctgaggtgggaggatcgctggatcccaggagatctgaggtgagccgtgatcatgccactgcactccagcctgggcagcagagtaagaccctgtctccaaaaataaaaaataatttaaaataaagcgcggaacactgtcaggttgtctgtgggggagcagcgggcaggcgaccaggagggctggctgctctgtgcagggctagtgcctgggttgggatgactcggcctcagagctgtaaataaagaaggaagagggccgggcgcggtggttcacgcctgtaatcccagcactttgggagggcgaggtggactaatcacctgaggtcgggagttcgagacaagtctgaccaacatagagaaatcccatctctgctaaaaatacaaaattagccgggtgtggtggcacatgcctgtaatcccagctactcgggaggctgaggcaggagaatcgcttgaacccgggaggcagagatcacagtaagccgagatcacaacactgcactctagcctgggcagcaagaacaaaactccatctcgaaaaaaaaaaaaaaaaagtagaagaaagaaagagctgccacagttgagagcttcctggatgcctttgttaactcttggggttttcacatacccatcttacagatggggaagctgaggctcagagagaggaaggcccaagtggaagagcaagcctggagcccaggtctcctcctgcctaatcctgggcttttgctggtggactcccgcccaatcttgaataacttcaggccttaaacaacctcccacgattgcttttcctcccattagaacaataaatgcagatacaaaaaccaactatgcttgtctacaaagcccaagagaatttttgaagtaataaaactgtcatgaatgcagactgtggtgattacatgacttacgaatttttcaaaactcagaatcgagccagatgaaatgatcctgcctgcaatcccagcactttgggaggctatggcaggaggatcatttgaggccaggagttcaagaccagcctggccaacatggtgaaaccccgtctctactaaaaatacaaaaattagctgggtgtggaggcacatgcctgtaatcccagctatttcagcagctaaggcaagaaaattgtttgaacccggaggctgaggttgcagtgagaaatgatcgtgccactgcactgtagaccctgtctcaaaaaccaaaaccaaaaccaaaaaaagaaaaaaaaacccttaagaatcaggattatacacgagcacacgcacactcacccataaattttaccatttgtagaaaagaagaagaaaattcaaaaccaaatacaatgaatgtactttctctggattccaattcacaccaaacaactgtaaaaatctgtttttgagacaattggggaacacaaactggcttttaggagatattaaggaatttcggttaattatgctagacttgataatgccattgtggttgggtaatgcccacataccatgccttggctgggcgtggtgactcacacctgtagtcccagcattttgggaggcccagatgggaggattgcttgagctcaagagttcgagaccaacctaagcaacatagtgagacctcatctctacacaaaattttaaaaaacattagccgagtgtggtggggtgcgcctgtaatctcagctactcaggaggctgaggtgggaggagtacttaaacccaggagtttgaggttgcagtgagctgagattgtgtcactgcactccagcctgagcgacagagcaagaccgtcaaaaaaataaagaaagaaaagaaaatgtaccctattcttctcttcaaggtgctcattgaagtatttacaaatgaaacaacataatacctctcacctgtaattttttaaaatttttatttattaattttttagagatggggtctcattctgttgctcaggctggagtgcagtggcatgatcacagctcactgtaacctcaaacttctaggctcaagtgatccgcccgcctcagtctcccaaatagctgagattacaagcactggccaccatgctttgctaatgttttaaaaattatttttgtagacacaagactatattgcccaggctggtctcagactcctggcctcaagtgatcctcccaacttggcctcccaaagtcctggaattacaggcctgagtcaccgtgcctggccaatttttttttttttttgaggtggagtctcgctctgtcgcctaggctggagtgctgtggcctgatctcggctcactgcaagctccgcctcctgggttcacgtcattctcctgcctcagcctcccgagtagctgagactacaggcccccaccacgcctggctaattttttgtatttttagtagagacggggtttcaccatgttagccgggatggtctcgatctcctgaccttgtgatctgcctgcctcggcctcccaaagtgctgggattacagacgtgagccactgcgtcctgccctgcctggcctattttttatttttatgttttttacagacagggcctcactatgctgcccggactcgtctcaaactctgggctcaagcgatccttctgcctcagcctcctgagtagctgggactacagggtgtgattttttttcaagtattcaaaaaaatggggaggggaaagaagatgaaatggcaaaatattgatggttcttgaacctgggtgatgagtatatgtaagcttattgtctcttgactttttgtgtctgtttgaaaattttcatgatataagagtttaaaaactgaaaataaaatttattgaaggagagtttgggccgggtgcaatggctcgcgtttataatcccagcactttgggaggccaaggcaggtggatcacctgaggtcagaagttcaagatcagcctggccgacatgacaaaacactgtctctaccaaaaatacaagaatcaggctgggtgcggtggctcatgcctgtaatcccagcagcactttgggaggctgaggtgggtggatcacaaggtcaggagttcaagaccagcctggccaagatggtgaaaccccgtctctactaaaaatacgaaaattagccaggtgttgtggtgggcgcctgtaaattccagcaacttgggaggctgaggcagagaattgcttgaacctgggaggcagaggttgcagcgagccgagattgcgccactgcactccaacctgggggacagggcgagactccttctcaaaaaaaaaaaaaagaaaaagaaaaagaaaaattagccaagcgtggtggcaggcacctgtaatcacagctactcgggagctgaggcatgagaatcgcttgaacccgggagacggaggttgcagtgagccgagatcgtgccactgcactccagcctggggtatagagcgaggctctatctcaaaaaaaaaaaaaaaaaaaaaaaaaaaaaaaaaaggagagtttggcgttcatcaagcaacatttggaaactacagagaactagcaaggcaaagacgcccctcccatccctaagtcctgtaccactatggttgttgacattttgatatatttcctttcagccttttctattctctgtgagctttttaaatgcgtgtgttgtgtgtgtgcacgtgtgtgttttacataactgtgtgagcaaattgtttatacagccctgttttttcccctcttaaaatgtaagctgttcccctgttaaaacatctttggataaacacaatctctaatgtttgcactatgtttattcaaaagggcagcattgccagaggttgaagctgtttccaatttcctcccattataaataatcccctcatggacctccttgtgtgtcccagaggagggattcttgggccaaagggtgcggaccctttggcagagctcatggtcaaaaatctcggcagagtttcagcctgacccgaagctaagatgacctcatacggttaaagtgagattggactgctatttatttcctgaacgtttgattcatgactcaggcttgtcaggggatcttttttttttttttcttctgtatgtgcttggttgcaagaaacagaaatccattcacactagctcacgtcaaaagggcaagagaatcgcttgaacgcgggaggcggaggttgcagtgagccgagatcgcgccactgcactccagcccgggcgacagtgcgagactccgtctcaataacaaacaaacaaacaaaaaaagggcggggggtgttgtatccttcagcaagtaaagctcatattcagcttggactagctgtctgcggagcatgttgttggggtatatctaggctatgttgattgttgaaatactgaattatttccgtattggttggtaaatcattctgtctcaagtcgctccaatgacgtcctccaccaaggccactctagctgccctggtccctctcccaggacttcccagacctctccacaatgcaggaagtaaagggtcaatatcgggcacagaagtgaaccatgctgctcggtcagtgcccaaagtattggttgtgaaattcattttttcttttcttttttttttttttggagacaaattctcactatggaactcaagcgatcctcctgccttggcctcccaatgtactgggattaaggtgtgagccactgcacctggactgcttgtaaaatatttgatgattcgcctagatgcaagtaccagaacagctgactcaaatggcttaaattgtaaggggaatctgtggctggaaaatccacacttgtgctggcttcagccaaagttgacacaatggctcagccatgtcaccaaggactttcttttctttctttccttctttctttcttctttctttttttctttcttttctctctctttctttctctctctcttttttttttttttttttgacgtagtctggttctgttgcccaggctggagtgcagtggcacaatcttggctcactgcggcaacctccaattccccggaattcaagtgattctcctgccttagcctcccgagtagctgggactataggtatgtgccaccacgcccagctaatttttgtatttttatttttttatttattatttttttaatcattcttgggtgtttctcgcagagggggatttggcagggtcacaggacaatagtggagggaaggtcagcagataaacaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccattcaaccctgagtggacacagcacatgtttcagagagcacagggttgggggcaaggtcacagatcaacaggatcccaaggcagaagaatttttcttagtacagaacaaaatgaaaagtctcccatgtctacctcttcctacacagacacagcaaccatccgatttctcaatcttttctccacctttccccattttctattccacaaaactgccattgtcatcatggcccgttctcaatgagctgttgagtacacctcccagacggggtggtggccgggcagaggggctcctcacttcccagtaggggcggccgggcagaggcgcccctcacctcccggacggggcggctggccgggcggggggccgacccccccacctccctcccggacggggcggctggccgggcagaggggctcctcacttcccagtaggggcggccgggcagaggcgcccctgacctcccggacggggcggctggccgggcggggggccgacccccccacctccctcccggacggggcggctggccgggcagaggggctcctcacttcccagtaggggcggccgggcagaggcgcccctcacctcccggacggggcggctggccgggtggggggctgacccccccacctccctcccggacggggcggctggccgggcggggggctgacccccccacctccctcccggacggggcggctggccgggtggggggctgacccccacctccctcccggacggggtggctgccgggcggagacgctcctcacttcccagacggggtggctgccgggcaaaggggctcctcacttctcagacggtgtggctgccgggctgaggggctcctcacttctcagacggggcggttgccaggcagagggtctcctcacttctcagacggggtggccgggcagagacgctcctcacatcccagacggggcggcagggcagaggcgctccccacatctcagatgatgggcggcctggcagagatgctcctcacttcctagatgggatggcggccgggcagagatgctcctcactttccagactgggcagccaggcagagaggctcctcacatcccagacgatgggtggcccggcagagacgctcctcacttcccagacggggtggcggccgggcagaggctgcaatctcggcactttggggggccaaggcaggcagctgggaggtggaggttgtagcgagccgagatcacgccactgcactccagcctgggcaccattgagcactgagtgaacgcaactccgtctgccatcccggcacctcgggaggccgaggctggcggatcactcgcggttaggagctggagaccagcccagccaacacagcgaaaccccgtctccaccaaaaaaatacgaaaaccagtcaggcgtggcggcgcctgcaatcccaggtactcggcaggctgaggcaggagaatcaggcagggaggttgcagtgagccgagatggcagcagtacagtccagctttggctcagcatcagagggagaccgtggaaagagagggagagggaaaccgtggggagagggagagggagagcgagagcgcatcagagtattttaacagcagaattgatcaaacagaagaaagaattagtgagtttgaagacaggctatttgaaaatacacagtcaggccgggcccagtggttcatgcctataatcccagcattttgggaggctgaggtgggcggatcacctgaggtcaggagttcaagaccagcctggccaacatggcaaaaccccgtttctagtacaagtacaaaaattagccaggaaatggtggcatgtacctgtagtcccaactactcaggaggctgaggcacaagaatgacttgaacccaggaggcagaggttgcaatgagccgagtttgttccattgcactccagcctgggtgacagagtgaggctctttaaaaaaaaaaaaaaaaagtccaggcgcagtggctcatgcctgtaatcccagcactttgggaggcggaggcgggcaaatcacctgagatcgggagttcaagaccagcgtgaccaacatggagaaatcctgtctctactaaaaatataaaattagccgggcgtggtggcaaatgcctgtaatctcagctactcaggaggctgaggcaggagaattgcttgaacccaggaggcagaggttgtggtgagccaagatggtgccattgcactccagcctgggcaacaagagtgaaactcttgtctcaaaaaaaaaaaaaaaaaaaaaaaaagatgaaagaaagaaagagaaagacacagagacaaaaaaagaagagaaagaaaaagaaagaggaagaaagaaagagagaaaagaataaaaaaaaagaggaagaaagaaaggaaagaaaaaaagaaaagagaaaggagggagggaaggaaggaagaaaggaaggaacgaacaaacacagtcacaggagacaaaagagaaaaagttttttgtatttttaatagagaccgggttttaccatgttggccaggctggtcttgaactcctgacctcaagtgatctgcctgccttggcctcccaaagtgctgggattacaagcgtgagcccccgtgcccggccagaccccatttctcccatttctttttatctctctgctttatcttttccagtggaagctcccaagatgactacctccagcctctggccacagggaaccttcttcatatcccacaagcaaaggagagcacctttggctcaggtttcctgccaaagtaatgaggctaactctgattgggccaacagagggcacgtgttcatgcaagagccaatcactgtgatcggagggacagaaggaaaaaccataatggccagattggctgagccaagtcacatgttcctcaaatagtaaggtgagtggctcacactagggctgcaggagccacacctgggttcaatttccattccaaacaaatttctgtcttctctgccctgtttcccgtcagtaacatagagataatgaaggcactcatatggtagagttcccgtgaggattaaatgagttagttaatgcaaagcactgagaatggtgcccatatacagaaagcgttcaaatgttagcttctgttatcgtcattacatggccattctcacctcttaaggctgcttttctttgcaagctccttatgctccacacatttccctctttctagttactattgcttggtaacaaatcactccaaaatatggtggcccaaaacaacaacagtcatcctattatttttcactgtgtctgtggtcagagttttgggaaggacccaactgggcagttctgacttggaatttcatatggttgcagtcacttggcagctgaaactggaacagggaagcagctgggggtggatggatctctctctctctctctttctcttactctctccccactccatgttgtctcagggcttctcaatgtggtctctccacgcgaactggtttgggctccctcacatcatgacagcctctgggcagttggacttacatggcagcttcggcatccagtacaagtgttttatgaaagtagcagccgcgactgggtgcggttgctcacgcctgtaatcccaacactttgggagcttgaggcaggtggatcacctgaggtcgggagtccaagaccagcctggaaaacatggtgaaatcccgtctctactaaaaatacaaaaattagctgggcatggtagcacgggcctgcggtcccagctactcaggaggctgaggcaggagaatcgcttgaacctgggaggcggaggttgcagtgagccgagatcacgccactgcactccagcctgggcgacagagtgagactccatcgaaatgaaagaaagaaagagaaagagagaaagagagaaagaaagaaggaaggaaagaaagaaagagagagaaagaaagaaaggagggaaggagggagggaaggagggaaggaaggaaggaaggaaggaaagaaggaaaggaaggggccttatctcttctgatctagccttggaatcatgcagcattacttctgccacatagtcttggttacaagcaagtcacaaactgcctagatgtaaggggagcttacacaaattccacttcccaatggaaggggtatcaaagttctagaggaatatatgggctgggaaacattgttgccaccatctttggaagttacatctggcttttactggtactgatttaagacccacctctatacagcctgtgcccaataacatccacctgggcacagctccaactccctgttctaaacagaaatagtttattgagaaatctcaatcccttgggatcaaaaacatgacagtttccatttgttctgcagtgactgctctctacctgctatctattgttttccaagactatgatccaaaatcacagcatccaagactgtgtcttttttatttatttattgttttcgagacagggtctcactctgtcgcccagactgtagtgcagtggcgcaatctccactcaccgcaacctctgcctcccaggctcaagagattctcctgcctcagcctcctgcgtagctgggattacaggtgcacaccactactgcctggctaatttttgaattttcagtagagacagggtttcaccatgttggccaggctggtcttgaactcctgacctcaaatgatccacccaccttggcctcacaaagtgctgggattacaggcatgagccaccacagctggccccaagactgtgtctttattattattattatttcgagacagagtctagctctgtctcctaggctggagtgcagtggtgcgatctcagctcactgaaaccttcaccttgccagttcaagccattctcgtgcctcagcctccggagtagctgggattacaggcacgcaccactacacccggctaattttcgtattttcagtagggacggggtttccccatattggccaggctggtcttgaactcctgacctcaagtgatccacccacctcggcctcccaaaggtggctcacagaagtgagccaccacgcccggcctgtctattatttttatttttaaaaataaatgtattgttaatttcctttttcctgttggataaaggctacagggaaacttaatgacaattttgctagagtctttaatatttctctcattttcattcatttaaaaacatagccttcaggccaggcacggtggctcacacctataatcccagcactttgggaggccgaggcaggtggatcacttgaggtcaggagttcaagaccagcctggccaacatggtgaaaccccgtctctactaaaaatacaaaaatttaaaaattagccgggcatagtggcatgtgcctgtagtcccagctactcaggaggctgaggcaggagaatcacctgaacccaggaggtggaggttgcagtgagctgagatcgcaccactgcactccagcctgagcgacagagtgagactcggtctcaaacaaacaaatacacaaaaaaacccacaaagttcatggggagggaaaggtgtagcctctcttgagaactttcctctctcattttccccccaaacaggatgatgcttcctttctcttctctatccttttctggacagccttgggtcctctcagtaacttatttgggtaagtggctggttataatggcagtgacctcgaacttaggctctggggtagttggccacttttgtcctcagttttgggcagtagtagcaattctggaagttctgggaaaaatccaatttgacatcatagtacgaacctgaaggaaaggtcttgatcccaaagagctctcggcagctcatggctgtgactgtcatccagggcctgggcctggggaaatgtttggggatctgtgggatcctttagggactgtcacagtgactcgggaaatgttgctataggcaactgttctggaactttttggtcttaggactcttttacattcttaatagtatcaaggaccccaaacagcttttatttgtgtgggttataacttttttttttttttgagatggagtttcattctagtcgcccaggctggagtgcaatagtgtgatcttggctcactgcaacctctgccccacaggttcaagcaatcctcccacctcagcctcccgagtagctgagactacaggtgtgcgccatcacgcctggctaatttttttgtatttttattagagacagggtttcaccgtgtcggccaggctggtctccaactcctgacttcaggtcatccatccgcctcggcctttcaaagtgctgggattacaggcatgagccactgcgcccggcctgtgtgggttatagctattgatatttaccatattagaacttaaaactggccatttaaacaaattttttattattgattgatttttgagacagggtctcaccctgttgcccagagtggagtgcagtggcatgatcacagctcactgcagcctcgaactcctggcctcaagtgatcctctcgcttcagctttctgagtggttgggactacaggtgtgcaccaccacacccaactaatttattgatttttttgtatacacagtcttgctgtgttgcctaggctaaaactgacaatattaaaatccagccatccacaagcacacactggcagagcaattatgtcatcacatgtcatggagccattgtatgctcataagtgaatgagattgaaatacgcaaaaaaagtcctggaattattatgaaagtaattttggccgagcgcggtggctcacgcctgtaatcccagcactttgggaggccgaggcgggcggatcacgaggtcaggagatcaagaccatcctggctaacatggtgaaaccccgtctctactaaaattacaaaaaaaaattagccgggggtggtggcgggcgcctgtagtcccagctactccggaggctgaggcaggagaatcgcggagcttgcagtgagccgagattgcgccattgcactccagcttgggcgacagagcaagactccatctcaaaaaaaaaaaaaaaaaagaaagtaattttaactttgtggacaaagtctcagggacctccaggaaaccctggaccaaactttgaaacttgctctaaaagcatttgtaagcctgggatggttaggtatgttcaacgtgcagccatgtaggagatagtgcatgtcaaaagggcccctttggatgggttaatatcactttggttcagttatctgtggcctggaaggggtcccttgtgtacaaacaaagctggggagtaggagggaagaatttttttttttttttgagacagagtcttgctctcgtcccccaggctggtgtgcaggggtgtgatcttggctcaccgcaacctctgcctcccaggttcaagcaattctcctgccttagcctccctagtagctgggattacaggcacctgccaccacacttggctacgttttgtatttttagtagagacggggttttgccatgttggccaggctggtcaaccctgacctcaggtgatccaccctccttggcctcccaaagcgcagggatttacaggcgtgagccactgcgcctggcccaggagggaggaatcttaaccataatgctcatcttgtactatctactggaaaccctaaaaatacagagaccctggagccactttctgggttctgctccccttcattgctttctagctgtgtgactttgggccaagttccttaaccactctgtggctcagtttctcttctggaaaatggagacaataatagtatctgcctctcagcgttgttataagagttaccttggcacacagtgttatgtgggtgtttacgctgagtattactgttattacagtaatcattcaaatccaagaagccatcaaaggtaagatgcattgttttttattttattttattttattttttatttttattttgagacagagtcctgctctgttgcccaggtgggagtgcagtggtgcgatctcagctcactacgacctccacctcccaggttcaaacaattctcctgcttcagcctcctgagtagctgggattacaggtgtgccctaccacgcctaagttttgtatttttagtagagatggggtttcaccatgttggccaggctggtctcaaactcctgacctcaagtgattcacccgcctcagcctcccaaaatgctgggattacaagggtgagccaccacacccagccaagatgcattgtcacttcaaatccccaggggagaaaaaacagctgccagttaaactatgacacataatcatttataagacacatcctgatttccaagtcattgaaaaggggtggggaggataaataagtcaattaaaaatggttttattcggcctggcgtggtggcttacgtctgtaatgccagcactttgggaggctgaggtgggtggatcacgaggtcaggaagttcgagaccagcctggccaacgtggtgaaaccccgcctctactaaaaatacaaaaaattagccatgcctagtggcacaggcctgtaatcccagctactcgggaggctgaggcaagagaatctcttgaacccagaaggcagagattgcagtgagccaagatcgtgccattgcactccagcctgggtgacagagcaagactccatctcgagaaaaaaaaaaaaaaagatggttttattctggaataggagagtatttaaggaaagaaaataaaaaataaaggaaaaaaagaaattttaaaattaaaaaaaatgatttcatgtgtacagaagaatttagcaacaaggatgtccaccacagcacttgtcataatggcaaacctgctgaaaccccaaccattatggattaactgcacaagttaagagagagccacaaaatattgtgactgaaggggttttcaaagattgttccaaaaccctggggatcctcaaaagtgaatcaatttctctctttcataatctttttcatcttcactctcctatgagtgcataaagcaaaaaataataagcaggtgacttctaaaaagaaaaaacctctggaaactatgcctcaaccccattctagagcctgctgggccactgctcagaaaaggaaatgtgtgccccaggctgacccactcttaagacaacccattctccacatgcctctgccttcagttacacagctgtacccagagaggggtcttgtctttactcagctgtccatccaccctccctgtgttgcaatgaatacatccctggtatcttctgtgcctggccccagcccaccccctcctcagagctccagccacacattccagtgccttcgagactcacctgctttgaaaggtagatccttacattcaattttcttgttgaaatacttcatgtggtttcttttttcttgacaagcctctgaccaacacttggtggtcattcttaaaccgttgatttttccccttcacacctattcccctccacgatcacaatcttcccagtctcaaaaatgtctctacctgggacctcaagccagaaacccaggcaatatggaggcacttcctctcctcattccacatatgccactcatggacaattcctgctttcctctaaggcagatgcttaccacacccctacccccacccttctatttagaaaatttctaaacatgcaaaagaattgcaagaaaagtagaagatataataatatactgtggatatggattcactaggtttttgcagttttttgtttgtttttgttttgagacggagtcttgctctgtcacccaggctggagtgcaatggtgcgatctcagtgcaagctctgcctcccaggttcaagcaattctccctgcttcaaccttccaagtagctgggattacaggcacccaccaccatgcccagctaatttttgtatttttcagtggagatagggttttgttacgttggccagctggtcttgacctcctgacctcaggtgatccgcctgccttggcctcccaaagtgctggattacaggcgtgaaccgtcacacctggccttttgtgtttttgtttttgttttggtttgtgtgtgtgtgtttgtttgtttgtttttgagatggggtttcactctgttgcccaggctggagtgcagtggtgtgatcacagctcactacagcctcaacctgctaggctcaagcgatcctcccttctaagcctcttgagtagctgagactgcaggtgcccaccaccacacccagctaatttttgtattttttgtagagacgggttttcaccatgttgcccaggctgttctcaaactcctgggctcaagtgattccccccaccttggcctcccaaagtgcttggattacaaacgtgagccactgtgctgggcctatttctttgtgttttaagaggcagggtctttctctgttgcccaggtggagtacgatgattcgatcatagctcactgcagcctcaaactcctaggcacgggagaccctcctgcatcagcctcccgagtagcccagactacaggtgtgtgccactatacctagctaatgaaaagaatttttttgtagacatgagttcttgctatgttgtctaggctggtctggaacctccagcctcaggcgatccccttgcttcagtcttccaaagcagtggggttacaggcgtgaaccacctggcctggtctcgctctttctctttcgacatctacattgatctgtagctatctacgtactatagatatctaaaagttatgtaaatgtctacttaagcttataggtatgagctactgtatgctatctaagcttataggtatgagctacaaattgctattgtgttatgaggggaattcaaaaagttgatggagaatggaattaagagataaaaattttaaaaaaatctttatttctcaccataaactccatcaaggtcaaagtcaagacacttttgtaaaccattataccagccatttagtctatccctaaataactgagggtcccaggaatttaatcatgtcagtgcagcctttttacattttctctttctttttttttttgatggaatctcactctgttgctcaggctggagtgcagtggtatgatctcggctcactgcaacctctgcctcccgggttcaagccattctcctgcctcagcctcctgagtagctgggactacaggcatgtaccaccacatctggctaatttttttttttttagtagagacgaggttttgccaagttggccgggctggtctcaaactcctaacttcaggtgatctgcccgcctcggcctcccaaagtgctagcattacagttgtgagccactgtgccaggccttttttttttgttcttgttgcccaggctggagtgcagtggcatgatctcggctcactgcaacctccacctcctgggttcaagtgattctcctgcctcagcctcccgactagctgggattacagtcatgagccaccacacctggctaatttttgtatttttttttttagtagagacagggttttgccatgttggtcaggctggtcttgaactcccaacctcaggtgatccaccctccttggcctcccaaaatgctaagattacaggcgtgagccactccgcccagcccagtctttttacattcttaactgaagaaaaatgggtgcccttcaaagattttaagattaggaaacaaaaagtcagaaggagccaaattgggactgccaagtggatgcctaataatttcccatcaaaacttgtaaaattgctcgtttgatgaggagaatgagcagaagcgtagacatgctggagaagaactctctggtgaagcttcccaggcttatttctactaaagcttcggctaactttctcaaaacactctcataataagcagatcttattattctttgaccctacagaaagtcaaccagcaaaatgccttgagcatccccaaaaactgttgctatgacctttgctcttgaccagtgtgcttctgccttgactggatcacttccccgctcttggcaatcattctctgattacgatttgtctttaggattgcacccgtgaaaccatgtatcatttcccgttacaatccttcaaggaaatgcattaggatctcgatctcacttgtttagcatgtccatggaaagctctgctcttctctgcagctgatctgggtgcgaccgttctggcacccatcgagtggaaagtttgtttagctttaatttcagtcagaattgtgtaagctgaggccgggtgaggtggctcaagcctgtaatcccagcagttcgggagaatgaggcaggaggatcacttgtgtccaggagttcaagaccagcctgggaaacctaatgagacccccatctctacaaaaaatacgaaaatttagtgtcaggcctctgagcccaagctaagccatcatatcgcctgtgacctgcacgtatacatccaggtggcctgaagcaactgaagagccacaaaagaagtgaaaatagccttaactgatgacattccaccattgtgatttgtttctgccccaccctaatgatcaatgtactttgtaatctcccccacccttaagaagtttctttgtaattcttcccatccttgagaatgaactttgtgagatccgccccctgcccgcaaaacattgctccaaactccaccgcctatcccaaaacctataagaattaatcataatcccaccaccctttgctgactctctttttggactcagcctgcctgcacccaggtgaaataaacagccttgttgctcacacaaagcctgtttggtggtctcttcacagggacgcgtgtgacacttagctgaacgtgctggcacgcacctgtagttccaactacttcggaggctgaggtggaaggatcgcttgagcccaggaggttgaggctgcagtgagccacaatctcaccactgtactcagcctgggcgacagtatgagaaccctgtctcaaaaaaaaaaaaaaaaaaaaagaaaaaagaattgtgtaagctgtaccaattgagatgtctatggtgttggctattgtttctgctgttaactgtaagtcctctttaattaaggcataaacaaatttaatttttttctgcaaaaattgatgtggatggtatgccacggtgggcttcatcttcagcatgacccaggctggagtgcagtggcgcaatcacggctcactgcaacctctacctcccagactcaagtgatcctcccacctcagcctcctgagtagctggaactacaagtgtgcaccaccacacccagctaatttatgtattttttgtacggatggggtttcaccatgttacccaggctggtctcgaattcctgagctccaatgatccgctggcctcagcctcccaaagtattgggattacaggcgtgaaccaccacgcccgaacttgtcccttcttaaaacgagttatccatttgtaaatggctgatttctctggggcactgtcaagtttttggaaaacatcaatgatttcatcattcttccacccaagcttcaccatcaatttgatgcttgttcttgcttcaattttagaattcgtgttgctctgataggggctcttttcaatctgatgttttattttagtgtctcaaactaggtcctgttcagacaagttataacaagttagtatgaatttattttggtgcaaaaaaaaaaaattgaactccatgcatagttttttcataatacacattttccatgaatttttggaagacccttctttttctctcttttttttttttttttttttttctgtagagatggtcttgttctgtcacccaggctggagtgcagtggcacaatcatggctcactgcagcctcgacctcctgggctcaagcaatcctcccacctcagcttcctgttactgggactataggtgtgcaccaccacaccttgataattaaaaaaaaaaatttttttgtagagatcggggtgggggggggggtctcactatattttccaggctggtcttgaactcctggcctcaagtgaccctccccaatcagcctcccaaagtgctgggattacaggcacaagccacagtgcctggccaataaatgttttttgaatgaatgaagaaattagtgcatggacatattttcccatcagctattacaataaccagactctgagttccctgaaggcagggcctgtgagtttcattgtgtaccctcagccctcaatagctgataaaaataaatattcagaaaatgcttgtaaaatgaagaagagaatatgtatgggacagaaagtagaccagtggttgcctggggctgggacagggaatagggagcgactgcaaatgggcagaagggatcttttcagagtgatggaaatgttcttgaattggctgtgatggctgcagaactgtaaaatcacccaaaatcagtaaatcacttataataggtgaattttatgacaggtagattatacctcagtaaagctgttaaaacaaaaccagtgttgagggaggaaataaatgttaccttggtaatactgaggactcagtagttatcaagacagctctcttccctgccctcatgaggttttctgtgcagggcggggacgccgacccattatagacagtgacagcctagagccatcacggcgggatggtggggaggggacaggcagaggggtgggaggccaggatgggggaagcccgggcagagaggttgggctggaatggggggcacaggcagaggggtcggggcggggactggtgaggtgcaggcagagggcaaaggggtcgaggtccagatgggagatgtacaggcagacggatccgggccagggcggggaggtacaggcccagttgaggagcataaggaaccataggagcccaaaggaggcgcccgaccagtcagggaggacttcccagaggggtgaatggctgagccagggcctgaagtataatgttcatgaatgcgacagcaatttttttttttttgagaccgagtctccctctatcactggagtgcagtggctcgatttcggctcactacaacctccacctaccgggttcaagcgattctcctgcctcagcctcccgggtagctgggactacaggcgcccgcccccacgtccggctaatgtttgcatttttagtcgagacggggtttcgccattttggccaggctggtcttgaacccctgacctcaggtaatgcacctgcctcggcctcccaaagtgctgggattacaggcgtgagccaccgcgcccagcacaattttaatttttcttaaacacttttcctgccttagcaaagtcttggaggggcggccgcccaggcgccgcgagcgcaagaccacgagggggcgctgcttcaccacgctagaggagcgctcccggcaaccacgtggccccacaggtgcgggaggtcggcgagcgcgcctggagggtggacggggtaggttacggccaggcagcccggggtggcgcgtggccctgcgcagggcagtggaagcagcgggccccagggagcatgagacgccgcgctccgtggggcggtcccagcgctgtgcagcgaggggccagggctgtccccggtcctgcgcggccggctcccggagaaactgtggcctcctgggggtggagctttaaaaaacaaaacaaaacaaaacaaaatcacccaacaggaagcggggtgggggcgcccggctctgtcacccgtggaatgcatcaaaatagcctgcgcggccgggggcggttccgggaaagggggcggccgcgaggggcggggctgcaacggagggcggagcttcccaaagaacgggagagaggagtgcagaaccggccccagggggacgtcctcctgttcccccaaaatgacctcgataggcgtttgtgtgggatggggttcctgggtagggcggggctttccagtgcgatgtccctggtctattcttagcgggctgaaacctggagaagtgggagtgggcggttgatccgggttccagggcctctttggagcgaaaagctccgtggagagccagagtctccatcctgggcgtgggagggaagatgggaaatgggccaagaggagcctgtgggaggtcctccccgggacggcccccttccatctgcatccgcgggaaccgttgtgggggcagctctgagaaaggctgctctgggcgcaaggaagggtggttgaacgaagagatcagtcagggacggggtgggggtgccttgactgtgtcccagatttgctgcgtgcttctgacctcagttccccatccttccagttgctcaaatcctctctgtcccagtcctaggccgtttctgcccctctcacttcctagtggtttgcagaggggaaacactgaggctgcctcttctgtgcctggccctgtgttggcacggggaagtgactgagacgccccagacccccttctccctcacagtccagtgaggcagacagacctgccaccagatagtgacagcccagaatgagtcattgagaggacgggggaggcacaggcagatgggtcagggctgggataggggaggagaggggcctgtaggatccaaggggagagtgggtgctgctgactcagtctgggggggttgaagagccgtctgaacttagaccaaaaaggtaggatggggccgggcgcctgttatcccagcactttgggaggcagagacaagaggatcgcttgaggccaggagttccagaccagattggacaacatagcgagacccccatctctacaaaataatttaaaaatcagttgggcatagtggtgtacgcctatagtcccagctactcaggaggctgaggtgggaagattggttgagcccaggaattcaaggctgcaatgagctatgattgccccactgcagtccagcccgggtgacagcgtgagacccctgcctccaaaataataatagcaaaaatactgggctgggcgcggcggctcacacctgtaatcccaacactttgggaagccgaggtgggcaaatcgctgaggtcaggagttccagaccagcctggccaacatggtgaaacccagtctctactaaaaatacaaaaattagctaggtgtggtggcaaggcatctgtagttccagctacttgggaggctgaggcaggagaatcgcttgagcccaggaggtggaggctgcaatgagatcacaccactgcactccagcctgggcgacagagtgagactctgtctcaaaaaataaataaatcctaatgaaaggataggatggggctcagggccttggaagagggtcaccttctaacaaaaggtatttgaacggagtcctaaagaatgagtagcattcttctgggaagtgctgtgtcaccatgaaggaaagggtgttctaggcagaaagaatgacaaatgcaaaagcttggaggtgagagtgtggcttatctctggcagggtcctctgctccctgcctgggacattcccccaacgtcataccccgacagagctgtcttaggtctcctagtcctggagtccacaagcaggttaatgggatgtaatggaaacgctatagagggcagacccctaggcttgttccctcacagcctgatgcttttttgtttttgagacagagtctcgctctgttgcccaggctggagtgcagtggcgtgatcttggctcactgcaaccttggcctcccaggttcaagcgattgtcctgcctcagcttcccaagtagctgagattacaggcacccaccaccacgcctggctaagttttgtatttttagtagagacggagtttcaccatgttggccaggctagtctcgaactcccaacctcaggtaatccacttgcctcggcttcccaaagtgctgggattaaaggcatgagccaccacgcccagccttttgttttgtctgcttttttagacagggtctcacgctgtcaccctcacaggctggagtgcagtgactgcatcacggaccactgcagcctggacctcccaggctcaagcaatcctcccacctcaacctctccagcagctgggactacaggcgtatgccaccacgcccacctaattttaaaattattttgtagagacagggtctcattatgttgcccaggctggcctcgaactcctgggctcaagcgatcctcccaccttggcctcccgaagtgctgggcttacaggtgagagccacctcagtgcccggcctcagcctgatggtttcacgactgcctttgactttggcccacactaatcaaatgtatcaccgtgggccactgactcaacttccccatctatccagtgggaatagaatattggactgcacaaaatcaaccacctcattttaaagagctataacgaaacaaaatttatatgttatatatttttaaaaatacatcttatagattaaaggactcaagaaacaatagcaagactgtaaaacgttgccacatcaggtgacactttggccagagttatggaaaatgctgacaaaacaagaatttgccagcatgggcaaagatatactgttaccaacccaacttgttaaaatattaatattgagctaattagaagtagtttttttttttttgttttttttttttgagacagagtctcattctgttgcccaggcaggagtgcaatggtgtgacctcaggtcactgcaacctctgctgcctgggttcaagcgattcttgtgcctcagcctcctggaattacaggcgcctgccaccacgcccggctaatttttgaatttttggtagagactgggtttctccatggcggccaggctggtctcgaactcctgaccttaagtgatctgcctgcctcgacctcccaaagtgctgggattacaggcgtgagccactgtgcccagccagcagttcataatatttgttttttattccttgattctaactctgtaaggaatagaaatggggtgattaattttggctcctttaatacccgcctggcaggtgctgagtcagtggtacctgttaccacgctatgactcagaggttatcactcagggacatccttctgactctgtttcagtccatgactcaggaggaaaggttctagggcctcaccagcgccctacacacaccctcgtttcccgaggggcaccattatattcttcctttattggctgtccttgtataatacaaatcttcaagtttagatacaaaaaaaatctggaaataaaagatagaaaagtacccgccaggcaccccttccccttcttcccggacccctccccagcgggcaccgacatgaggtaactgcgcatcttctcctcctcctccctccccgcctcctgggcccccaacttggccctagcccccatgagaccttggcccaaggaagacatgggccccaaagttggagggtgatggaggtgagtacagcattggcagtgaacgcagcgtgtgggcagagggcagcccttagcttgtgctgtgcatggggggaggagacgaccagatttggggtgggcactggtgtggggcccaggagagcccctaagcaagtgtcccccctccatcctgtccaacactggaaggtgggggtcaacagaaactgggaaatggagtgtgaaccaagggcattcagtgggattgtggccaacccctgtgtctggggttggggaagggggcattgccagcctaagtccggcagtgaaatggttcccttagccaggctgggtccgtccctgaattccatcctgttgcaacctgggcgtcacaatgttggttctgtagtggttgtcggggtggttgagggaggggggtgttgagagggtgttgaggggcgcagccccagggagcagggttaggggagaggttgtgcctccacccccaggcgtggggcacctggagatgatggagtgggtggggcggctcgggctacagttcaatctcgaatgtcttttgcaggtcgccagaaaaggggttggagggtttctctacagtggctttgccttctaatgccgcccactgggcctcaaaggggtccaactctggagctggggcaggcgctggctcagggggccagggggccccattggggcgagggcgggcctggctcccaggggcactgggtatggcagggggcgggaaggccccagctttcccaagcagagtggcaggctgaggctggagctgggcggctgagcagaaggcgtttgccaccatctgtgagggtgtgatgcccaccacgggcacccggggcatcggtgggtagcccaagcccgggtaggcgggcacaaaagggggctgcatgtgtgggggtggcaggaacacggccacttgggcaggtgcagcgtcaaagggccccacgggggcggggaaaggctgcagggcaggaggcatggtgggcactggggccactgaggctgcttgctgctgttgctgctgctgctgctgctgctgttgctgttgctgctgctgctgctgctgggccttggccacctgtgacacctcctccagccatcgctcagcctctgaaggtgtccgcttgtgcccaggctggaaggcagctgcagggggcacggagggctcaccccaggcagaagtccctggagagaggagagggacaggtgagggagggggcaacggcttcagcagaagaaccccaggctgggaactggcatggtgtggggatcagagcacggactctggagccagaccgcctgcctctgaatcccagctctgcacctcccgagcttgctgacttcaggcgagtgacttcaccattctggaccacgtaatggttctggagtgctaagaacatacaattctgagcttctaaggccctatgatgagaaagtctaagtaggactgacatcctcctagccccactacccttctgactggtcagtgccaagcctaggattcactattttgttttgtttgttgtctgttcgttttgagagtctcactctgttgcccagggatgccctatctgggctcactgcaacctctgcctcccaggttcaagcaattctcgtgcctcagtctcctgagtagctgggattacaggcgcgcaccattacgcccggctgattttttgtatttttagtagaggcgaggtttcaacatgttgcccaggctggactcgaactcctacctcaagtaatccccctgcctcggcttcccaaagtactaggattacaggcatgagccaccacacccagctggtattcactattttgaatatcacccttgattttctaataccttcagaatgtattgttttaggagttaagattctaagattgcatggatctttctccacatgtataatagtatcctaacatagtaaggtttgacagtttagggttgggctttctaagattctcgagttctacatttacagtgtttgagattctaaggtgcacctgtggttctaggataagcccatggctctaagatgatacatggttctcaaggattccatggttctgaaacgatctctgggctctaaggtgatcccaagattctagcatgagcacatgcctctgaggtgaccccgtggctctacaatgatcccgtatctctaagataattcatggttctaaagtgatcacttgattctcaggtaatcccacggctctaagatgatccttggttctaggatgatcctctggttctaagatgatctgtgtttctaaggtgatcctatggctctaagatgatccatagttctaggatgatcccatggttctaaggggatcccgtggctctaaaatgatcaagggctctgagatgatccatggctctaaggtgatcacatgattctcagataattccatggctctaagatgatccatggttctaggataatcccctggctctaagatgatccctgggtctaaggtgatcacatgattcttaggtaatcccatggctctaagatgaaccacggatctaggatgatcccctggctctaagatgatccatggctctaagatgatcacacgattctcagataatcccatggttctaggctgatcccctggctctaagatgatccctgcttttaaggtgattgcatgattctcaggtaatcccgtggctctaagatgatccatggatctaggatgatcccctggctctaagatgatccatggttctaaagtaatcgcatgattctcaggtaatcccatggctctaagatgatccatggttgtaggatgatcccctggctctgaggtgatccatggttctaagcctgtaggatcccaagacccctggagtgtctgggtagccctggatagaggaggggatacaggagggacatgcagggtgtctgcccagcagaaagcctggctttacctgttgtggcaggtggtggccctggtgctggcgctccagcactggcaaaagatgaactgatctgtgtgcacagagcgttgatgctgtcactgtcgccggcaccaggaggctccatctcaggcactgggaagcagggcaggacagaggtcagcaaacaggcccagaccctgccgcagccccgccctctaccccccgccctcggtggccctctcttctgacctggatcctttcccactgtagtaactaagtggccatgaccgccaaataactgtgcatatgtgtgtgtcacatctgagcggcagaacgactgagttagtgtgtgacatttgacggctgacagtaacagggaccccaaaccaaggacctcaagatttgtgtgtggccacaaaccagaagagttgtctaaaacacttagcaacatatattagtttaaaaatatgcccaaccacgtgcatggcaaggacatgggcccagtctgaatgggctgtggcatggccgctcactggctggggatcatttgtccagctttgggcagagaagtggggtcctgtccaccccacccaggctgatctatggcaaacatcatgagagcagagatacacctgtgccagcaaggcaggagacactgagcaggcatggcggccccgtgagggcttgtgatgagcaaaggtaccatccaaataaacaattaaggactcccatgtgtggtgtgtgacacatgtctgtgatgttttagccaatggctgtctccaagggaccatgtagatgctccagtgaacatcagaactggccgtaggagtctgcaggtgtgctgcgggggcgcccgggtgggacagccaatgtcgacaatgattgtgcatgttcgtccaaacaggaatgcagcttctgagagtgtgactggggtccgggtgtgtggcgtcctgatgcgtgactcccaggtgtgagtgcatctgccgtgtgatgtatgagtgtgtttgcaggtgtgcatggatctgcacatgtggctttggagtttgcataaatctgggtgtgtggtctgtgaaggtgtgtaaatctgagtgtgtttcttttttaaaaatatttatttatttatttttgacacagggtctcgctctgttgcccaggctggagtgtagtggcacaatcttagttcactgcaacctccgcctcccaggctcaagcaatcttcctgagtagctgggactacaggtgcatgccactgcacctggttaacaataaaggttaatttttaaaatttttttgtggagatgagatcttgctgtggtgcccaggctggtctcaaactcctgagctcaagtgatcctcctgcctcagcctcccaaagtgctgggattacaggtgtgagccaccacgtctggccatgtgtgacttctgactctgtgattttggagtgtgtgtgacttgagagtatacctgactgtgagtttgtgacttctgagcatgtgtgacatgtgtgtgggggggtgcaatgtctatgtgagtatgtgaaatttctgtgtggcaagtcacatccaaataagcgtgcagctggccacaggagtgtgaggcttccaaataaagcctggagctcctgccacctctcaggagcacgtgtgacagcctggggagtgtgcctgatgtcaggctgcgttaaggacagcaaatgtgagggtgtgtgatggcccaggaatgctgcaggcatggagggagcgtgcgtcactctcaacaagggttggcatctgctcaccggttaccttttggagcagcccagcctcctggatacagctcaacaccctcccacatcaatccccgaggacataacgggtaggcgatgcagatttggcatccaggagcaagagagtgggccagaccacatgggttcactggctgctggtggggaagacagcccttctgcctgcctccctcaggcaaaaactcagtagcgtgccacctctatcttccttccctatggcagagattgctcttaatttttttttttttgagacaggttctcactctgttgcccaggctggagtgaagtggcgcaatcttagctcactgcagctgggctgaagcaattctccttcctcagcctcctgagtagctgagactacaggcatgcgccaccacaccaggctagtttttgtatttttttgtagagacagggtttagccaagtgatctgcctcaagggatccttgagcccctcttgggctcaagtgatctgcctgccttggcctcccaaagtgctgagaccacaggcatgagccactgcacttggctagacatcacttttagatcatgatgtacaccacctgtctcctttccctggcccatggcagacattagtaatcaaacacaatgcttttttcccccacggagctctttgctcaccttacccgtcacctgtccagacctcatggctcccctggccaagtgctagaagaaacagatccttgtaactccatcttccctccaagtaaaacaggacctttatatctcctctctcttaccaccagccatggcagacaatgccaatcaattatgatgtgctgctccttttcgtgatgggtactgcctctgctcaactctgtgcctacaacaaatgttcctgatccatcactatgcccctgacccctgggatctctgagctcactggtgccctgcatgtcatgtttctactgaccaaattctgggagtctcccctttgggtaaaaacataacactcttgctactctttcttctcccttgcccatgacacacactgcatggagatcatgactgctaaccgatcatgatgtgtgccatctttctcgtttcccttaaccacagcagacattgctaattgatcatgacttgtgatgccttttttgccatcccttgatgctccctgccctaggcagacatttctaatccatcacaatcctcctttcctcagagcctgaggaatacctgtatccttcacctagccagatctcataggtgaatgcttggagggagggagcattaacctgtagtgcttcagtctactcctgagcaaaaacaggccaatggtgttgcttctcttctttccctggcccatggcaaacttccttatccatgcccgacttctttgtccatagccgactttgctaactaatcatgatgtgtgtcatctttctccttttccttaaccacaccagacattgctaactgatcataacttgtgatgcctttctactatcccttactcagagcagacacggcttatcaatcctgatttgtgctatctctttcctctcccttgcccactgctaatcgatcatgacatgttcccactctctctcctttgcccatggcagacagtgcaaatcaatcacagtgtgaaccatctcgcctccatcccttgcccacatcagatagtgccaattgattccaacgccgtttcccactgggcccagggctcacctgtgcccttcacctggaagtcagtgcggcgctgcagcgtggatggcagctcattcagccgtaggctcagctgccgtttgaaaggcgagttcttctggctgagtgctgggaacccacggaaggagccctggcgaaccagctgctccaggggtgcatggcgccgggggatggcggccgcagtggtgcctgcagccacaggggtgcctgcctcacccttctcaccaggggatgtggtggctggtgtcggggacacgtgcccaggctgggcagggccaggagccacagtgggggcagctgctgcctctgctggagacatgggggagatggatggttagatatctcaccatggcatgcaaggcctctcccacctggttctcttgatcattctccaaggctgccttccttgcccagtgagtcctgcccttaagacaagctagtcaaagccctgagatatccctcacccccacccaagtcatgatccccttccccaggaggctcttggaaaaccgtcccccaggctcaccctctgtgtctctccagcttcctgcccctttctgtcttgccctgctgctctcaccattcttccctctaagaccctctatctccctttttgatcacttaacaaacactgatttggtgctggaacaacgtatcaggcattgttccagatgcttttcaagtattcactcattcaatccacacgactacccatggggttgatatgattctcattttacagatgtggaaactgagacccagcaaaggcccagtgtgatgtgcccaagtcctactgctaggaaggggcaggctgactctggaccccatgctccaaatgactgcccgccctgtctccctctgacctgtctctccatatcatgttgtctttctctgtcactgtctgttcccctctgtctcccacctcccagccttcttcccacctcaagtagctcctcccttcctccagtcattctagacatggcttggatgcttcatcctctgagaagctttccagattgttccaggctctcagcctggctccctccactctgagactgttctctgtactctgggaggaaacctgacctccccactcagcaggagaggcaggctcttcaagggcagggctgccctcacccctgccaaaccacaaacacacatctaccctcctaccagggcctccccgagctgcagcctgaatgccatggagattaaactcaggcccagagagaccctcccataaggatgtcgcccagtgctgcttctcccatcgtgacacaatggggcacccatgcaggcctggcccaaggcagtggaaacagcccaggacaggttggaaccaggaaacctcctctgcctccactcactgtgtgacctcaggaagtcccctcccctctctgggctccagtttgccaactagaaacctgggataaacatctcagtgctacctcccaagcctctgagagggtcagtttaggtagtgaatgttaaatgttttggaatccaacaaaagcatgcctggacacagtggttcacgcctgtaatcccagcactttgagaggccaaggtggggggatcagttgaagccaggagttcaagaccagccttgccaacatggtgaaacaccatctctactaaaaatacaaaaaaagattagctgggtgtggtggcacacgcctgggattccagctactctggaggctgaggcaggagaatcgcttgaacccgggaggcagaggttgcagtgagccgagatcgcaccattgcactccagcctgggcaacagagcaagactctgtctcaaaaaaaaaaaaaaaaaaaaaaaaaaaacttagctggacgtggtggcgggcacctgtaatcccagctactcaggaggctgaggcacataaatgtcttgaaaccaggaggcagaggttgcagtgagctgagatggcactactgcattccagcctgggtgacggagagagactctgtctcaaaaaaaagatgttcttttttttccccttacaatccaggatctaattcaggatcacatctcgcatctattaatacctgttaaacctcattagtctcccttaatcaggatcagttcccaatggtaaattctttgtaaattctaagtagtgttttcccaaagaacttaaattcacagtaaaataaataaataaattaattaattaataaataaattaattatatggtgactcaggacatacatacaattatattttaaacacacacacacacacacacacacacacacacacacacagagttttagaacttacaaagtatttaaccatttacctagaaagttagttttaccaagtatgtttcaaactttcttgatggcaatccagagttaagagatatcttttatatggtagcctcttacacacgcgtgcacacacacacacactctctctctcaaataaaagtatcattaaattatatttaacccttatgccattcaatatactctgatattttctatgattctctatttcattgtttttaaattaatttttttttttttgagacagggtctcactctgtctgtcacccaggttggagtgtggtggtgcaatcgcagctctctgcagcctctacctcctgggcccaggtgatcctcctgcctcagcctcctgaggagctgggaccacaggttgcaccaccacgcctggctaattttttaatgatttgtagagacagggtttccctatgttgcccaggttgatttcatttttttaaatgtcagccacatgggctgggcccagttgtccacacctataataccagtgctttgggaggctgaggtgggaggatcgtttgaggccaggagtttgagaccagcctgggaaacacagcaagatcccagttctacaaaatataaaaataaaattagccagatgctgtggcttgtgcctatattcccagctacatggaggctgaggcaggaggattacttgagcccagcagtttgaggctgcagggagctgtgatcgcaccactgcactccagcctgggcaacagaattagacctcaactcagacaacaaaacaaaacaaaaaatgccagccacagtacagcaaattggcttcccaggtcttaatacacaggttgaaaagccctgagaaggccaggcgcggtggctcacctgaggtcaggagttgaggccaacatggtgaaaccccatctctactaaaaatacaaaaattagccatgcctgtaatcctaactactcgggaggctgaggcagaattgcttgaacccgggaggtggaggttgcagtgagcctagatcacaccattgcactccagcctggttgacagagcgagactccgtctcaaaaaaaaaaaaaaacaaacaaaaaaagaaaagcctcgaaaagccccgaattggtggatagtaaaccacttggtgcgcccttagaatattgcccagagaactctttccatggaagttgctttatttatttatttattttttgagacagagtctcactctattgcccaggctggagtgcagtggcgtgatctcggctcactgcaacctctgcctccagggttccagcgatcctcctgcctcagcctcccaagtagttgggattacaggcgtaggccaccatgcccagttaatttttgtattttagtagagacgaggttttaccatgtcgcgcaggctggtctcaaacttctgacctcaagcgatccgcccatctcggcctctcaaagtgctgggattacaggtgtgaaccatggcgcttggcctgaagcatcccctactttgtaaactgttgagtctctgcacctctgcagctccctgccccctgatactgggtaccctgtgcccactctgtgctctgctggcgcttggggcaacacccacctttcttcttgtccggggcctctcgctcagcaggccgcccacccccagacaggcggaaggagccctcgcgggcgaagctggtgcggctggcatcgaaggcggccgtgaccccacattccttctcccgtcgctgttttcgctccaggcaggcggcaaaagcacagcccacagcgtggctcagcctctcgccctatggggagaggatgggcgggggggttagaggcgctgggcagtgcggacaggggccaggggcactgggttatagccgctaggtactgggttgccccggatgagtctctgcacttctctgggcctcagtgctcatctgcaccacaaggaagggctggaaatgacagctccaagtgagagaagccagaaaccaatgagcctgttgtgtggctccacttactggaaattccagaagaggcaaaactaatctatggttctatggtgaaaaaaagtcagaacaggccaggagtggtggctctcgcctgtaatcctagcactttgggaggctgaggcgggaggttcccttgagcccaggaggtcaaggctgcaagcagctatgatggggcaactgcactccagcttgggtgacagagtgagattctgtctaaaaaaaagtcagaatagtggtagtttcctatggggtggggaatgactggaaagtgtatgaggggatgaccgggtggtggcaatgttctagatattgataggggtttcgttacatgggtgtatgcagttgtcgaaattcgttgactgtacaccaaaggcttgtgcattgctatagactaaacgtgtgtgtctctctcagttctaacgctgaagccctcatccacaatgtgctggtatttggaggtgggcctttgagagacaataaaagttatgttagatcatgaggtggggtcctcatgatggaattaatgcccttatttatttattttttttctgggacagggtcttactctgatgctatgctggagtgcagcggcatgacctcagctcactgcaacctccacctcccggcttcaagcgattctcctgcctcagcctctcaagtagctgggattataggcatgcgccaccacacctggttaatttgtgtatttttagtagagatgggatttcatgatgttggtgaggctggactcgaactcctgacctcgagtgatctgcccacctgggcctcccaaagtgttggggttacaggcgtgagccaccatgcccggccgggattaatgcccttaaaaaggaaaaagagacaagagatctcccctccaccttgagaggtgaagatacagaaagcaggcatccatctgcaaaccaggaaaagagccctcaccaagaaatgagccatttgacaccctgacctcaggtatccagcctctagaactgcggcaaacaaatgtttgtcatttaagccacacagtctacactatctggttatagcagcctgaactaagacaatcatatctttcgctttgaagaaaaaaaaaatctggaaacaaatacgaattctacttaatggtgtgcatgctgaagtagtccaatgcctacaaatttgacatacaaaaagaatcagacggtgtgactgcagtcgctcacacctgtaatcccaggaggccgaggcaggcaaatcacttgaggccaggagttcgagaccagcctggccaacatggtgaaaccccatctctactaaaaatacaaaaatctgccaggtgtggtggcgtgcacctgtaatcccagctactagggaggctgtggcaggagaatcacttgaaggttggaggcggaggtttcagtgagccaagatcgtgccactgtactccagcctggggcaacagagcgagactccgtctcaaaaaaaggaatgaaccactgatacaagcagctcacgcctgtaatccgagcactttgggaggccaaggtgactggatcgcttgagtccaggagttcgagaccagcctgggcaacatggcgaaatcccatctctacaaaaaattagccaggcatggtggcgtgtgcctgtagtcctagctactcaggaagctgaggtgggagaatcgcttgaacccaggttgcagtgaggcgagattgcaccactgcacaccagccttgatgacagagagagacgctgtgtcattaaataaattaattaaaaacattgccgggcagggtggctcacacctataatcccagcactttgggaggcagaggcaggcggatcacctgaggtcgggagttcaagaccaacctgacctgcatggagaaaccccatctctactaaaaatataaaattagccagctgtgatggtgcatgtctgtaatcccagctacttgggagactgagacaggagaattgcttgaacccgggaggcagaggttgcagtgagctgagattgctccattgcactccagcctgggtaacaagagcgaaactctgtttcaaaaacaaacaaaaacacaacattacactaagtaaagaagccagacacatattatatgattccattaaatgaaatatccagaataggtaaatccacagagagacaaagcagattagtggttgccagggggctggggtagtggggagacggggtgaattgatgaatggagatggggtctcctttagcggtgatgaaaatgtttcaggactgctaaagtcaatggttaaacaacactgaaaatgtattaaatgccattgaattgtacacttttaaatgattaattttatgtgaatttcacctcaagaaaaaaaaaaagttgaacttggggaattctactgcttaatggttaaaacttccttcagaagtaggaaggaattcatccaacatcacattctttacccacaggagatggtcctgctcttttctggctttcattctcatccccaaatagttttttttttttgttttcttgagacagagtctcgctctgtcacccaggctggagtgcagtggtgcagtcttggctcactgcaacctccgtctccagggttcatgtgattctccccacatgtgattctcctacctcagcctctggagtagctgggatcacaggcgcctgccaccatgcctgtctactttttttgtatttttagtagagatggggttttaccaggttggccaggctggtctcaaattcctgacctcaggtgatctgcctgccttggcctcccaaagtgttgggattacaggcgtgagccaccacgcctggccaatactcttttttttttgagatggagtctcactctgtctcccaggctggagtgcagtggtgcaattttggctcactgcaacctccacctcccgggctcaagtgattctcctgcctcagcctcccaagtagctgggactacaggtgtgcaccaccacacctagctaattttttgtatttcagtagagatggggttttaccatgttgtccagggtggtcttgaactcctgagctcaggcaatccccccgccccagcatcccaaagtgctgggattacaggtgtgagccactgcacccggccctcacgtgaatactctaaattgggaaccgtgagtatactttcttctccagatgaggaaactgggcacacagaggttagtgatgtgcctgaatgcaggaagcttgtaggggtggggctgggatgccagggcatgggagggaagagttggccagctgtggcaatactcacggagtccttcagtgccagaaaacagtggcagatccagcggcgggtagtcccgtcacgacagatataggagaaagccttgtccaggttgcggtcaggagcacaaaaggagaccttttcgatggtctggtcgaccagaagatcctaggaggggccggggaggccaggagaagagtgtgaactctctttcaagtgttcactcaacattctctctgtgtctgctgaaatccagtcctgaacagggtggggacccagcagtcactgagacaccctgtgccttgcctgcagagggctcccaggtcagcgggaagatcagacccatcacgagatgggcagcccagagtcatagccaggatgtgatgaaggaggcccaggcagaggggtcagggccaggaccatggaggcacaggcagaggggtcagggccagcatggcagaagccagcttggggtcagtcaggcaaggcttcctggagcaggtactaactaagaaaaaaagtcaaggaacagtattttgggctgagggaactgccttgcaaaggtggtgatcttgttctgttattaacccaactctcagcatgtcctgggcacctcctagggcagtctctgggctgggggcagcagattcaaggtgagcaaacagatacctagattgtggcctcatagtgcttccttctgactgggcagggtgacaataaacacttaatggctaacattcattgatctgagcagttacgatgtactagaccccatttaaatatatatatatatttttttcccagaaggagtcttgctctgtcacccaggctagagtgcagtggcatgatcttggcttactgaaacctctgcctcctgggttcaagtgattctcctacctaagcctcccaagtagctgggattacaggcacgcaccaccacgtctggctaatttttgtatttttagtagaggtggggtttcaccatcttggccaggctagtctcgaactcgtgacctcaggtgatccaactgccctggcctcccaaagtgctgggattacaggcgtgagccaccgtgcccagcccacatttaaatttttttaaaaattattttgtatagatggagccttgctatgtttcacaggctgatcttgaactcctggcttcaaggggccctcccagccaggcaccatttcaaataacttgcatatgctttctcacttaatggtagggtctattataatccctgtttttctgcgatggagtttagctcttgttgcccaggctggagtgcaatggcatgatctccgctcaccgcaacctccaagtctcgggttcaagagattctcctgcctcagcctcccaagtagctaggattacaggtacacaccaccatgtccagctaatttttgtatttttagtgagatgaggtttcaccatattgccaggctggtctcgaactcctgacctcaggtaatctaccctccttggcctcccaaagtactgggattacaggcgtgagccaccactcccggcctataatctgcatttttcagatgacacaactgagactcagagaggttaaatctcatgcccagggtccctaagctgggatcggagctgaggcagctgctccagtctctctcacccacagcctatgccagttcccaaataatcacagcatttattcctgagttatgacgacagagggagcacacggcatcgtctagaaggtccctgagggagggatgtgcagaggaggcgggaaggtgtcctccgccctgattccagcagggtgagcagacaggccccctggcgtccacccccacaggcctcctaccttggttttgtcgtccaccactcggagcccatcggctgacacccacaggacagacttcacggactttcggcccatctggagggaggcaaggggacaaaggagccgggtcagggtgcctctccctgtctgaccttgccccctccctcatggcagccccctcactcaccgccttcagcttcttcaccgcatcttcacacacgtgcattccccgggactcctctacctccacgtgacccaggtacttgggttggagggaatggggggggggacatgaaacagcacagtaatcactcattctcagtgtccactgagcatctgccatgcagtagacaatattttaagcacatgatgtgtatgcaatcatttattctgcatctcaactctacaaggtggagaccccattcctggtctagacatgcagatggggaaactgaagcacagcacgtagtagtgattccagtctagctatacccctgggccctaacccggcaccgtggtcctcccgagagctagtatttgcatctctgtgcgtgaggactggccacaggccatgcaaagctgctttgcctaccctaaagcagctagaaagagcctgggccagtccccacaatgactgaagggagttagggtacctgagccccagctccctcatctgcaggtgggatgactgttgcctcgagtttcccaggggaaagctcagttgcacactgctgcagttttaataacacaccctttactggctgccttactttcctcactcccaacaccatttcctgcacctcccaaatgaagccgtcacattcaaagtctgcttccagggaagggaacgtaaggtggtggaggggctgtgaccatggtcagtcatcacccagccaggaaggggctgagctccacagtccacgccgtgaatcctgctctccccctcacaggaatgtaaaagcgtagggtatggctaaaccagaacgtccttatcggtagagcgctttgcaaatgctacaccacatgacggcttccaggtaatagtttgctggtacaaaaaaatgtgttctggttttttgtttgttttcaagattgggtatcactctgttgcctaggctggagtgcaatggcacaattaaggctcactgcagcctcgaccttcctggactcgagtgagcctcccacctcagccttgtgagtagctgggactacaggtgcgtgccactgtgcctattttttgtagagacagggtctccctgtgtttgcccaggctggtcttgaactccttgactcaagcaatcctcccaccacagcctcccagtgtgctggaattacaggtgttagccaccactacgcttcaagtttttttttttttttttttgaaacagagtctgctctgtcgcccaggctggagtgcaatggcgctatctcggctcactgcaatctccgcctcccgggttcaagcgattctcctgtctcagcctccccagtagctgggactacaggcgcccgccaccacgcgaggctaatttgttgtatttctagtagagccggggtttcaccgtgatagccaggactgccggcctcacgctttttaacaccaacttcaaacgacttgggctggtgtcccagccaggccgcgcacccgcacagcacagcacagcggcccccgtccccctcgccccgccgcaccctgccgcgcccactcaccctgaccgggaagctgcacgtgcccttccgcaccgcgtcctcgtctgcctgccactggtgcgggcgcgacgcctcgggcacgtaggctggcttcctccgccgcaggctctgccgtaacttgttcatggtgcccgccccgtctggtgacacaggacagtgatgtgggtcaagggtgggggtcagaaggtatggagctcaacggggagggaccagtgctcagggagcatggggtcagataacaagataactggaagcggggttacagggtcagttggcagcgcacatgggatcagcagctcaagtcaggggacagtgaggaaaggagccaacagaagccaggggagtgcgaaagttgggcactggggattcagaaccatggggctaaggggctggaggtctggggtggggggtatggggcacaggcttcagggcattggagggcttcttgtccatgtggtggatgcttcctcattcccagggccgtgaggacacaaagggtgggaaatcagacgtaggagggcatgggggctagagggcatagaagtcaggggccgcacggtcaggggttttaggggtatgagagtcaggcagtggggtgatcagagggcagcagaggagttgggaattgcatgggtcaggggtgcaggagtcaggtgcatggggtatgggggatggtggcttgtctctggaactctgagacacagccattctgtgacaactgtgtctagtccccaggcaaggcttctggggccttgcactgatgactgtcagaggcagggctgtgggtgtgtggagtgggtgtggaggtggccagccactaggacagtcttcactgatcagccatcctgacagtcccaggactgggagcaccaacagcagagggggatgtgtgtgtgtgtgtgtgtgtaagcaagtgtgcaatggtatgttctggaacgctgtctatgaggggctctatatctgagcaggcaagtaatgtctgtgagtgtgtttctgaaggctgtctggaagaacgcatgtctgagaatggtgtaggatagtgtgcatctgtgaatgctgtccgtgagggatgcatccaaaagtgtggttgttgtctgtgagtctgaggccctgaaggttgttgctgaagggcatcgatgtctgagagtggttgtgagtgtgtgtctgctgtctatatccgagagtgccgttgttgtctatgagaacttgtggctcagtgcctgttgcctgtgagggtgtgtctgtgtttgagatgaggctgtcgtctttcaaagggtgtgtccatggctgttatccatgtagctatgtctctgtgtgaaggtgtggctattgtctgtgatgacattgcctcggagagtgcatctgagggatctacaagactgtctgtgtccaagagtgcagctgttggcggtgagcctgtgtgactgtggctgttgccttagagtgtgggtgtgtgggtattgcacagagggtgtatctgtgtgcagtggtgcatccgttagggtgtgtgggaacatgaggttgtctttgagagtgttttcatgagggttatttgtaagggtgtgactgttgcctgagagagtgctgggtgttctttgtgagactgtgtgcatgtttgtacacaaccgtagctccctgtgagagcctgggcccgcgggtgctgtctgtgaaagtgtggctgtcgtctgtagaaatgtgggtctgtgtgtgttgtcagactgtgcaagagtgtgtctcttttttttttttttgagacgaaattttgctcttgttgcgaatgaaggctggagtgcaatggcatgatctcagctcacagcaacctctgcctcccaggttcaagcgattctcctgcctcagcctcccaagtagctgggattacaggcatgtgccaccacccctggctaattttgtatttttagtacagacagagtttctccatgttggtcaggctggtctcgaactcctgacctcctgacctcaggtgatcctcccgcctcagcctcccaaagtgctgggattataggcattgagccaccacgcccggccaagagtgtgtctcttgagagtgtgtatctcagagtctgtatccagcgagcgtctgtggtcacctgtgaatctgggtgttgtctaggaatgtgtgtgtgtgtatatatgtgtgtacattagtgtgatgaacatctacttcagcatgtgttcaagtgtgtcactgcattagcaagtgtgtgtgatctgaaagagatctgtgtgtgtgtgtgtgtctctaaccgtatatctacagatgtgagtgtttgtgactctgcacttgaggctgtaagggtgtcagggtgtgggtctgtgcatgagtggggctctgacagtcgctgaggaagatgcctgagggtgtccactcatgagtgtgattaagcctgtgtgaccgcagggacagctcctctggtcgtgttaggcaagaccacatacccattcagccctgccctgtcccaggctggtcgctcacctggctccgtcctgcaggtttctgggggccccggggccccacagggggctgggggcaggtgccgctcaggcctccggggtccgccctgcccgagtaggggaggagaaggtgagaagtttgtctgggttggggctcagtctgatacttcaccccgactttggacttcggccagccccctccatcttgggctccctgatgagagtcctagttgtcctagcaactgttaccagggagatcagccacctggttccaagggcccaggaaggagtaggtatgtttggggggcatatgttggggatgtgctggcctcaggagagcctcactgcccaaggggatgctgtgagagtggccttgccctccagatgcatgtgcagggctacacacatacgcagccaactcatatctacaggtgtccctagcatctggccacagctttacacacttggttacacatagacgcaatcacagctacacacctcagatcgcagatacacagaaacagtcccaaacccagaaagctgccactgtaaacatctggccgcatattcatgttcggacacacacagtctctactacagacatttgtccacatacatgcagcatgtcctggcccactcagttcccaccgcagacacctggtcacaccacccactgaaatttggtcacttccacattctcagccacatacaaagaacggggcccagacaacccactagagccgcacatgcgtggccacacccagtgacagctatgtttcctcaggcacccgcccaagatactgacgtcacaggcccacccagatagaatcacacccagagttcacccatttgctgacccagtcccacacgacacaatcgcagctatatacacttgttacacgtatctccactgctgcacactgacatctggtcacagatgcacacggacagacacacatttggtcccacggagaccacatgcagcctcaaccagaccagtcacaccaacacactgtttttggccacacactgtcacgtggtcacacatactcagtcataggtgctcaaatctggtcacagcatcagtcacacagtctattgtgacagccacactcatagggacagtcctctatacacaaccacatacagaggtactgatggccacagcatgcaaccacccacaaacaactgtccccaaggatggcctccctcaagacacagccatggtggtcctataggcatggccacagcatcaatgtccatgtgacccagccacctatcatggtcataatcccgtcacccagtgtccatacacagctgtaccatgtcactccaatgcagcgaaacatatagcctttcttcatgcaggcacacaagtcacacagacaagtatgtggccatatagaatcacagccccgtgtggacaggtgcacaaagtcacaagccccacctacacacatatctcacacatagagacataagcacacagttttagtttcccatgtcacaagtacacagtccagtgtatgcacataaatgcagtgaccaggatgtggcatttatagctgggtttacacagaagacacaggtcctgacggagccatgtgcacagtcaggggccaatctcatcaccaaatcacacattctaacacaggtagtccagcacatacacacagagacaaaaacaggcacacagctacagaccccacactctcacagatgatgacatagacacagacatctatagtcacccagggtcagacatgcaggtcaaacacaataatatggaagccataatcatatacacacccctacaaacataatttcctggcaccagcacggagacacatccacagctttagtcacacacaaataatccctctaaatagccagggtttcagacaccaagtcaaatacataacccaaatcacacatgcaaggctagaaatacacacaatcactcgttatcacacacactgctagatacagtcatttatattaatacaacctcagtcacacactcagacatggttctgcatacgcaaccactgtcatacacaatactgtcacacaaccccagtcacacagatacaatgtgacttcagacacaatggcagtcacacaggcagagttgcacagacaggtgtgtgtcactcacgtatataaaatcaggtagagacacaactataaacattgtcagtcactgggagggtcacacatgcacatggagccatagacaactccactcacacacagtcacaactccacccacgtcctgtcagtcacacgcatgcccacccccagactcaacacatactgtcacatacccagttatagttaccgaggcatacaaacacagggtcacaaaagacacaatcacagacaaccacagtcacaaccatgcacacctccaaacacagacagactaaaaagtacaggattctgcacagtgagcagctgtcacacacacatacacagtcacacagatactagggtcacagacacaatcatagacaccaccacaagcacacacacacgacacaggattcctcagtcaccagctgcagcgtacgcgtgcacacacacacacacatacacagccacagtcacacaaacacagggcacactcctagccacctccccatcgcttgcttccccccttccccccaggtaccacacacaaagccgccgtcaccgtcaccgcagacactgcagacagacagggctgcacccggtccctggggagccgcccccagcccagcctgtatcctggagcctgagggccaggtccaggagggggaagcggtgcattgtctgcgggtggggccactcttggacagctgggcagccctggggacaaagggggtggcctagggatcctgtgccctgggacccatgaccaagccagggggtggagggattgggggttgcctgaaattgtccttattttattcaggctggggtggggtggagtcccaggcacagggaccttgttttgagaagtggctgtgcctgggactccgcccaaggattggggggatgctgtgcccagggtgcctctgagacctgggggcaggctgtgcttggagtccccctaggccttggcgtggtgggaacgctgtacccagtgaccccatcctcgagaccgtacctaggcagttgttgtgctgagacccccttaggcagggagaggtagggattgtgtctggaatcgcgtcctggaggccttggtttgatacgggggctgtgtccccgaccccatctccatccccatctagttctaggagttgtacctagaatctctgtacccctgtccttggttttgtggggggtctgtgtccaggatccctatttccagggccttggcaagagatggcccaggggtctgtgcccaagaccccggtctcctgggctttggcttgttgggcctgttcctggacccccacccgggaccaccctctccagccttggcaccctctctccatccgggcgccgcggccgcctcccaccctccgccacatcagcagcggcgccgcccccgacccgagcccccctctcccgcccttctcacgctggccgccgcgctgcgggacatccagggcccgggcgcccccgcctcccgcggccctggctgggcctggctccccgactgctgctgctgcggtggtggcggcggcagctcggtctgacgccggccagggcccggggccgggggatggtgcgggatgcacgcgcgcgagcctcctcggctcccgggaggattccgttccgggggcgggggagcgtccctcggcctcggctccccgccccgaccccgcccattggcccgccgcctgccgtatatgcagaaaggtcccgccccgccccgccccttggcctgaagaccccgcccacacgcagacaccatcccgccttccccggcgcggccaccgccccgccacgcccattggccagtcccgagctgagcatgcctatgaacatcgccgagtcctttcccccggtccgtcctggcctgggctccctttccctccgtgacacctgacctcagatcccctccagctccgcgtccgaaagagttgcttctagaggcgccttgccgccccacacccccaaatcttcccgctaaccccatggttccccagcccggcgggggcgggcaggagcggtgacgaggcactttttgcaaggaaggggagggggtgtgaacagaccaaggcgtcctgatgcgcgatcggttcggctttgggaacgggggcgccggggtgggaccagggcgcggttctggccgctacggcgcccccgccgccccctacgccaatcctgtgggtttggcaaagcagctggcagggacattagagcccattagtaaaggcgaggttgccaggagtgaggggagggagcccgcagccaagaggcactgtgtgtgtgggaggcctcagctgccacagcccttagagcaggtggtcagcacgtgggggggggtggggaaggcgagaaggaggttcccggcaacagatggcacaggcagggctgaggctttcagacaagggggtgcaggggggctggccatctgcacctggaggccgctggcgagctcctccccctctccccccaaccctagcgcagagaaaagagctgggggaaggggagggctgcttttattccgattgaagattcagctcccccccaccccgcacccctacctctccaacacactgagaggctggaacccgggagaaggcagctgttggaggagcgagggaggcagaggtgaggactgcctccaggaccatctcgccttccacgggaggcttgaggcagaggtgagggctccccctgatgagtctgatctgcataacgacatgcagcggcccaaggctccctcaaatgttggcaaagataagttaaccgcttccctggcttagttttcggatacccactttcgagtggggaggtgcaggatctagggcacgaagttgggagttccccagccccagggatcctgagctcctgggccaaggaggagagccaggcaagactgggaagcccaagggggctcctctgacccagggcagacggggaagggataagaggcactacagcagggtacggcctgctctggggactgaatcccccatggaggctgtcatgagggatccacccaggagtcccctttggtggggaggctgccggcagtggctgcgtctggctggcgactggcccagtagcggtggtaggtgtcctggaagaggtccctgcaggcgatgtgggctcggaggtgggcacaggccaggaaagcccctgccagcttgcggtgcagggcataggtctcctcgggtgggggacacagccggtgccgcagcagcaccgggatgaggtcctgtatgcggcgggccgtttcccccgacccaaagtcataagggccctgggtggcgaaaggctcccccaggatcatcactgcctccacgtgggcgtcggagaatgcctgggagtgggggtggggggagagcaaaggcagccagtgtggacatagagccctctgcataacccagaaacaacgtgtagcctccactccctccagtctccaccatcaacacaagccccgactcccccaagagtgagcagcttccactccttccagagcctggactagacaagcagctgcctattccccgagtaagcaccccttacttccctgcagagcctccaacagacaagtagccctctactctctctacaacagagaagcggccttcgcccctcctgaacagatccacggtgcccatgttcccagggttcctgagaagtcaggcagccacctccccccactcctccccaaagcccccagcatggataagcatccctcctagagtcacccactccccaggtatagacacacagcctccattctccctagtggagacaagcccctcctagagtcccccagtccccgggtatcgacatgccccatcacctactcgagttcctcagtggagataagcagccccccactgcaccccaccaacagacaccagcccctttctcccccagtgtctccccaccttggtttcaaagcctgtgaggaatttgaggtccctggacttctgcaggacacagtctctgtctccatcagctgcagccttcaccacctggggagacgggtgggagttagagggacaaaggccggggctcaagggggctctggagaagaggagctggaagcctgggcctcccatgtacccacctccattctcatctgcctgggccctccctcacctggaagcttcctgctcccaactttgcctgtctgtgactcccttaaagactcttccagagcctgggagtggaggtggttagggatcctgcccacctagagggcaatgtggggcctgcccgggaactgccacagtgaaacagtgcaagaaacaagtgtcagggctgctggtgagtccaaacactcaactggacgggtatgagcgtgacccagaactgcatccaaagttgccaggggccatctctctgtctcccacactggactttgagccccgtgagagcaaggatcccagttgggtttagtcgcacggggcacagggtctggtgctgaggaggccctgggtgaaccttgctttagcaaatgagcgtgtggagcagagcagctctccagccatgagctcacacacacagggcaggagttgtgggcatggagagatgcccagctggggccagctggctagggttccgtcccagctctgccacctgccggcctgttgggcctccactttctcatctgtgaagtggggatgccaaaagtgccttcctcatggggctgttgggaggataaagaaggtgatgcctgtggcctgcctggcgctgtgtctcatgcaggaagcaccgtaaatactataatctaccattattaagaggtgggcacgggggtccctatttcacagaaggggaagcagacccggagtagggagtggaaccgggtcacaccactcactactgggttgcccttttttgtgaggagggggattcgggattaattccattccctctctgctccccctgcccgagcatgtctggccatctgtcaggcctgtggtcctcagaagccagggcttttataaagtggcaaaggctgcaggcctgggtgggaggggagggtgtcagtggggagaagaagggtctcagctttgtgcctcagtggccggcagagagggccccaaggggcctgtgtgtaaggggttatcacctcctcttcctgttcctgtctcagaggtggccccaccagtccccagaccagacccctagacacctctgtccccaacttctagcccgtctgctcccatggactgccagctgggcatctcccagccacccctctgcccctctccacagctgccctgagctcccacccccaggcttagagaccacttcttattcagagggagttttcaaattctgactgcatcccttccctgctcaaatcttttccacggctccgcagggcacctggatacagtacgagctgtagagcgcagcctctaaaatccgcagtgatttgctgcagcctcgtcactgtgctgccccccgacctgccctcccactcagaatgggagcttcctttgggtgctctagggccttactactaccagtgtggtccccatacgggtagccctggcctcacatgggtgcgtgtcagaacatagcatcttacacccacctccagaccagaatctgcacttgcacaggatcaccaggcttcacatgcacatccaagtctgagaaatgctgctctaatgggactgtcccttatcactgcctcgtcttggcacatgttgtctatctgaaataccctccgagccttttcccagttggcccccctcatccctcaggtctcaatgctgacactccctcctccgggaagccctccttggctccccaggctggctcaggctcctcctctgggctcacagtgccctgcgcttcccccatcccagccctgtccatgctaggtcctcactgtcgcctcactgactatgagttctgtgaggaggggcctaggctgggcccaggtcactgctgtgtctccagctttgcctagaatagagctgggcacagaggaggggtcagaaaacatttagattggctgggcaggagggctcatgcctgtaatcccagcactttgggaggccaaggcaggaggatcacctgaggtcagcagttcgagaccagcctgggcaacatggtgaaaccccatctctactaaaaatacaaaaattagccgggcgtggtggcacatgcctgtaatcccagctactcgagaagctgaggcaggagaatcgcttgaacccaggaggtggaggttgtggtgagccaagatcgagccactgcactccagcctgggcgacagagcaagactctgtctcaattaaaaaaaaaaaaaagaaaagaaaaaaaaggaaaagaaaacgtttagattgatggctacatgattagaggacccagggcaaatgtccaatctcaacaacgaatgaaatggtattctctgcagcctctaagacaaactgaccccagtgggtgggagagacgagccaagtacgtgaggaaacacctcctgtagacagagttgcccccagggacacacacctgggctggacaccagggccctggacagcatggggtctggagaggtgacaagatggtcactgtgggcctttctcttaggagggagctgggcagtggttggaagcttgggctctggacctggaccacctgaatttgaatcccaagtctgctgtgatcttgggcacgtgacttaacccctccaggacccagttacttcatctggcctaaaacgggcctaaaaacggtccttttcgcattgtgttgctgtgtggtttatatcggttcctatacgtaagcactaaagatggagcctgacatattaagtgcttggtgtgtgctagttgctacgagtattactattatttattatttcttaattccagaaactgcatttcgccttcttactcctctgcctcagtatatggcttgagcctcagcctttcagaggccctggggtctgggggagactcacctcgatgtaatggtctgtgaactctgtcccaaactcccggcttgcaccaaagtccagcagggtcacctggaagcaaggaatggtgaaaggaatgctgggatcccattcacaccctcactgtctattggggcagccaggatctgtctccctccccatgaccctgcctgctcctgccagagtccctgcctggctgcctcactgggctcctggcctcatctcccgtttccaaccgaccccctagctgaaggcttttttttcttttagttctaattgtttatgcaataacaaattttagacccagaaaaaaaatccagagaataataaaagatatagaaaagtaaaatgaatactcacaaatcctgccacccagcctaaaatataaaatactccccatccggccacacgtggtggctcacacctctaaccctagcactttgggaggccaaggtgggtggatcatgaggtcaggagtttgagaccagcctggccatcatggtgaaatcccgtctctactaaaaatacaaaaattagccaggtgcggtggtgcgtgcctgtaatcccagctacttgggaggctgaggcaggagaatggcttgaacctgggaggtggaggttgcagtgagccgagattgtgctattgtactccagcctgggcaacagagtgagactccgtctcaaaaaaaaaaaaaaatactccccatccacgtggagcctcctgtgtatcccgccctgatcacacctcccccaggtccccagggtcacctctttcctacaatcattattaaattatttcctgaaattctcatttatgcatttctatattttctatgtatgtgtacagctgtaaacaatctagagtaatggtgtgcatattttcaaacttcacagaagtggtatttactgtcgttctcctgcaacttgtgctttttgcccagcatggttttgcgatttatccctgttgccatgtgtcgctccagctcagtcactcatttcacactgctgtgtaaggacttcgttgcgtgaatctatcacagtgcatttatctggctgaggaatactgtggttgtttctgattttttttccttctttttaaatatgtttattagttataaactctgttctgacatttttttttttttttgagacagggtcttgatctgttgcccaggctggagtgcagtggcacgaccatggcttactacagcctcaaactcccacgctcaagtgatcctcccgcctcagcctcctgggtagctgggaccacaggtgcaccccaccacacctggctaagtttgaaacattttttgtagagatagggtttcactacgttgcccagactggtctcgaactactgggctccagtgatcctcccacctcagcctcccaaagtgcagagattacattagtgagtcatctcacctggcctgtttctaatttttgtgctcaatgaacacctgtaagaatgtttctagggcagtagagtttgaactgaccacaacccacagtaagaaattgtttctactggtggctgggcgtgatggctcacacctgtaatcccagcattttgggaggctgaggggggcggatcacctgaggtcaggagttcaagatcagactggccaacatggtgaaaccccacctatactaaaaatacaaaattagctgggtgtggtggcgcacgcctgtagtcccagctagttgggaggctgaggcaggagaatcacttgaacccaggaggtggaggttgcagtgagtcgagattgtgccactgcactccagcctccattctcaacaagagcaaaactctatctctaaacaaacaaataaataaaattatatatatatatatatatatatatatatagagagagagagagagagagagagagtttctactgggcgttcatgcaaatgcacacaaaaacgtcaaacaaaagtgtcccaaggccaggcgcaatggctcatggctgtaatcccagcacttcgggaggccaaggcaggcggatcacctgaggtcaggagttcgagaccagcctggccaacatggtgaaaccccatctctactaaaaatacaaaaattagctgggcgtggtggcaggcacctgtaatcccagctacttgggaggctgaggcaggagaattgcttgaacctgggaggcggagggtgcagtgagccgagatcacaccattgcactccagcctgggtgacaagagcgagacttcatctcggggaaaaaaaagaaaagtgtcccaaaataatatggatccctcccatctgtgacatactgacatcatctattccataaaaaaaaaaaaagtgctggtggatcatgaggtcaggagttcaagactagcctggccaaaatggtgaaacctcatctctactaaagatacaaaaaattagccaggtgtggtggcgggcacctgtaatcccagctacttgggaggctgaggtgggagaatttctcgaaccccagaggcagaggcttcagtgagctgagatcgtgccattgcactccagcctgggcaacaagagcaaaactctgtctcaaaaaaaaaaaaaagtcctgttggaaaccaataaattgatgtcaggactcctatcacttggagttgggtaaacatatccctggggtatgaacctaggaggggacacagggtgtatgcacattgtggagaaagaagaggcaagggactagggctggagactccgtggggccacaggatgctcagcaaagggttcagacttgattctgagggggctgtgaagctgtaggagggatatcttgaccagatttgtgctttagaaagacccctctggggacaaaatggggatagaccactttggaaaacagttggcaacccctgataaaggtaatgatgtatccaccaacactcaagtctgcaggtccaaaccttcactacaggtctttgtacaccccctgccctctgctgggaacactctgctctctggatggttgtctccttttcccctgtcccatctcagccttttttttttttttaaatagagcccaggctggttttaaattcctggcctcaagggatcctcctgcctcagcatcccaaagtgctggaattacaggcatgagccaccatgctcagcctctggtctcagctttttttttttttctttggtgagacagagtctcgccctgtcacccaggttggagtgcagtggcatgatcttggttcgctgcagcctcgacctcctgggctcaagccatcctcccacctcagcctctcagtggcctcagctttaatggcacttgctttaagaagcctcccttaatccctgctcaccccacagtgagtcaggcactttccctgggatcccataacttcctgtgcctcccccaactccagcccccacccctctgcctgtgcctcccccatcccggtcctgacccatctggcctgtgctcctcattgtggttggtacgaaccactctaggtcatcgctgtgtggtgccaggtccccccactggactgggcactctgagaaggcaagggtcagggatgtctttttattgctgtcaagaggcaagggcaggtgatggtgtgttctgtatggatgacgagtgtggtagtgatggctaactgccttccaaaagctgtacttcccctctgtgctgtcacattgccaccaggaaccactgcctagccagggactccattttccagcatgccttgcacctaggcaggctatgtcactagctctcactgatggaatggcaacggaagtgctatgtccattgccccaggatgagcttagtgtatttagcctgggcattactttttccacttctggccaaagcagttaggcagggggctaccctcaacctctcttaccctttccttgggaagcagagcgttgcaaggccccagggatggcaaaaccacaaaagggaaggagctgggtccctgaatcccacttggaaaagggccacccctgcctattgtgggggtaatcaactgctattttgttgagctaccgaaatatcaaggaatatttgttacaacagcaaatgtacccagacacacatcactaggaaccaacctggtggctggaggcatcatacaggaagttggcccagttggggtcagtctgcatgaatcggaactcaaacagctcccgcagacacagcgtcaggagctggaagcaaatctggggtggggagaattaacaggcatctcagtgtgatctcccttgtggtgccactgctggcctggagagagaccatgagcctggcccacccgccctggggccatgcccttcccactgtgccaccagacagcatacctggttccgcaggtcctggcttaggccctggcactggtccagggggacccctccagccagctccatgcccagcacccgtgtcgtgcacagctccttaaccacggctgggacccggaagaaggggtcatttgccagcagctgcctggggcagaaggaaagggaggaaggggacttcgtgcttcaggcttgtgacccagcttgagacctcctccttgtgctctcagaagtcctccatgaacagtctgcgccatgccctccctcctgcccatctcttgctgctgtctgccctgggtgatgaccaacacctggctggactgagatttaacaagtatctccaaggtgcctactctgcacctgcctctcaactgggtgctgctggaaacccagtcaccacttccctacccgaaacccttctatgacttctggttgccccagaaggatgagttcaggtgtatttcagcctttgcattactttttcattaatttattaattcattcatcccaaaagtagtcactggtatcaaaatctgtgaagtctatagtgatacccatgggtcagtccttgcaatcactactctgaacacaggtaaatagcgggaaagaatcttctgtgtccatccttgtgctaaatgatgctggggacagagcagtgaatgaaccagtccctggacctggcccccaaccccgagaccacactctcacaaacagacggattatagattgaggtaagtgtgttggctgggtgcagtggcttacacctgtaatcccagcagtttgagacaccgaggtgggagaatcgcttgagcccaggagttcaagaccagcctgggcaacatggtgaaacctcgtctttatgaaaaactacaaaaattagctgggcgtggtggcctgcgcctatgttcccagctactcaggaggctgaggtgggaaaatcacttgagcctgggaggtcgaggctggagtgagctgtgatctcaccactgtattccagcctggaaaacagagcgagaccctgtctcaaaaaaataaaagaagtatgtctatgtaagatttgggaggcagagaatatgagcaaggtctctggttgaggatccgtgggatggctggaggagagtgagggagaacaggagggtggtgtggggttttatcctaagggcacccgggaatcctgtgggggacgggggggtctctggcccatcaccccacttcacactctcttccttgagctcctcctttggggtttgtatttttgtccttgttgataaaaatctctgatgtttgaactgctcctcatgaaccggagccatgtggggcatttaatcggcacactcccctttcatcctcactccacagtgaggggatgctctctggaggccactgtacagattgtatggatgcaggccacgtaacctgttaagggctgacccaggatttcatcccgcatctgagtctagggtgtgtgcctctaaccactgccttctactgagctttcactctctgtctacagagcttggcacgttttaggctgtcaataaatgtgtgagccacactgtggaggaccccactgaggtgccattaattgctacacatcaggatgcttccaagatataaccacattgttccacgtagccatagttcatgcattttcactgctacgtaatattccacagatgaatataccgttgcttatttgtccaccctgctggataaacttgatggatatgggttgtttccagtttggggctgttgcagacaagcgttctagaacagtctgatgatggatactggcaaaagggtccctagagtgttcagcagggagggcaatcgttggcttgcagagttcttgaatataagggcttatgtgatgatgccaacctgttctcaaaatgcttctaccaccttctcccacaacctgcagtgcagaagggatgctgtggagcccactctctctacctcccagtgctgtgggctccttgctctccgtcagtccagcagaggtaagagtgtgtcattgtgtaattaattaagtgcctgtgtcggaccacagtggatatgcggcccatctgacacaggagaagactgccgcttgggaagtgacctgcccaagctcacagaggacaaagggggtaagcgaagtgatggatgagtggaaggatggatgaacaatgattgaaagaaagagagagtttgccctaccccacagctccagctgccagaagctgagggggcagctacttcccacccagcctgggagggagggaaggagggaagctcagggcactcagctcacctgaaattctgggcacaagccgcctcacgacggtagtcacactcccaagccagctcctgctgcaaggcctgcaggctctgctcggcaaacaggcctgtgggggaggtgtgtcagccagggaagggccccgagcgcccactggtggatgccttctcctgccaaccctctctctcctgtctcccgcgctgtccctcacagctcctactctaggcctgtgacccacatctgtccctctcctgtctcccacgcagctcctgctcctgcctctgacctgcatctgtccctctcctgtctcccccgctgccccttgcagctcctgctccaggcctctgacctgcatcgtatcatctgtctacacatttccctcgtccttcatagcaccacagggttccccggggtcttccgaagccttttctgggctctgtctcttgcacatgcctgcaccccagtccctggcagctctttcagagtcacgcctgcctctattctgtctccttttcatactgactctcacgttgctcactgcatctgaatctcacttctgggcctggctttccctctccctctccgtctcttacacaaacacaacccaccctcctttctctaggcgcctcctcacgtgccatccccaggactgtccttttctgctctgccgcacctacaatcccgctccctgcatgcacaccctctgagagtctgctgctgtatcttggccctgggctcccgtctctctaaggctctgttaaaatccataaggcttttcccacacctgctcatgctccctgggctctccccgccttggtgtcctttctgtctctgacactcccgctcacaacgccagcacaccctgcccgtgctctcctctgggctccccctcctgctttctctctctgggcatagcccctttgggaggtcagcagaggagtgggtgggcgcctcacccgcgggcagggccgcgctcatcttgagtaccgccagcaggttctggacatcgctctgaatgctctgggctatgccggggtactgtaaagggagaagggagggagagaaggtgggagtcacttctctgggctagcagggtgcccgcccctaccctgcccagcctcccctctcacctggatcttcacggccacctccgtcccgtccctcagcaggccctggtgcacctgcccaattgaggcagcggcaaagggcacctcctccaaggaggccaccttggcctgccagtccctgccgagctcctcttcaagaactctctgcggggagtggtcacagccatcatcattgtggcagagttcattccccaggctgagtgctttacgtgttgataaccccagcactcaccacatgcagggccctgggctaagctcttggctgccaccccctttgcagatgaggaaactgaggctcagagagagctcccttgtgcaagtgactagccaggcactggcagactgtgaggctcagcctagcctgtgtcccctgaacctggactccctgggctgttgtggggattgctattatttatagttaagactcataacaatcctttttttttttttttttttgagacagggtcttgttctgtagcccaggctggagtgtagtggcactctcacggctcactgcagcatcaacctcccgagctcaagccatcctcctgcctcctgaatagctgggactacagatatgtgccactgtgcccagctaattattactgttattatgtttagtagagacaaggtctcactatgttgcccagggtggtctggaagtcctgagctcaaatgatcctcccaccgcagcctcccaaagtgctgggattacaggcatgagccattacacgtggccaaccatcctttattatgctcttatatgttattattaactcacagttactgtgatcaagaagagctaaaatttacaatgggcttcctgtgggccaggtcctattcagagtggcccagagagaggcagtacctgcctggggccgcatggcgaataagcagaacagggatgggagagcaggtgggtggcggaatcctggattccatgccttacccagttattagaacagctcatgtcctgggcacttacttggtgccaggcgccgtgccaacctattacagacacgatctcctttaattaaaaaaacagtaataatcacagctaacacctgcatgttgctcacagtgaatcaggggccgttctcagtgctcagtgtaaactgatttatgtgaccctatgatgtgggtgtggctgtcactatcgccatttcccagggaagggaactgaagcacagagaggtcaggctgctgttccctgttttaatcaccacagcaatgctaggaggtgtgtactctggtgattcccattttacagatggggaaagtgaggctcagaggcagggagtggggcagtgaagccaggaaggtcaggcaggtcagaggagatggagcctgcctccctcaccgccctcccccactgggcactcaccagcatctgccagcggggcatgaagtcggcgctctggcggacccgctcaaagatgtgctgcagctgagggctgatgaagctgttgtcttgggagacagtggaaccaggggggaagtaagcgaagaggtgaggagggaccccgtgtgagtatctgaagttggggcctgttggtgggtagggctggggtagaagggaggtcaggggtcaggagtcgagggtcatgctgtaccctggatgctgagcatctggccaaccttgagggcggcccctcgaactgtacataaggtctgcacaatccgctcggcattggcctccgacaggaaggggctggagtccagcccagaaccaccctctggggagagaacaaccattagaattataaccacaaatatcatcactgcggccaggcccggcggcccacgcctgtaatcccagcactttgggagacccaggcgggatgaacacaggagtctgagaccagtctgggcaacagtgacatcctttctctattaaaaaaaatatatatcatctgcacaccaacaggaatggctaaaacgaaatacggaggccaggcttgttggctcgcgcctataatcccaacactgggaggtcaatgcaggaggatggcttgagtcaaggagttccagaccagcctggataacaaagcaagaccctgtctctaattagctgggtgtatggtggcatgtgcctgtagtcccagctactcaggaggctgaggcaggaggattgcttgagccctggaggttgaggctgcaatgaactgtgatcgcaccattgcactccagcctgggcaaccaagcaataccctgtctcgggaaaaaaaaaaaaaaaagaaaagaaagaaagaaaaataaacataagttaaaacaaattgcaggagagtcaaagagtaactactacagtattgaatagatgagctatcactacacacaacagcccaggtgggtctcaaaaatgtaatgctgagtgaaagcagccgggtgcatgaaatcatcacccagttacactgatataaagagcaaaacaaggccaaacaaatccagggtccaggagccaagacagaggtcacccttggtgaggggagtgaccaggtggggacatgagaagggcaacgattctcgaaatgtgggccagggacccctgagggtccctgagaccattcagaggggtctgagagatcaaactatttttatcgtaacattaagatgttattttcccctttctgctctcatgcgtctaagaacattcactggagttctccagaggctccaggacatgtgaggacaccatcagactgacagctgatggaatgtgtacgtgtgtactcttgtgttttaaaaatatctctgtatttttgttgtgtttttgagacagtgtctcactctatcacccaggctggagtgcagcggcacaatcatggctcactgcaacctcctgggcccaagtgatcctcctgccttaacctcctgagtagctgggactacaggtatgcaccaccatgcttggctaatatttttactttctgtaaaaatggggcctcgctatgtcactcagactggtctcaaattcctgacctcaagggatcctcccgccccagcttcccaaagtgttgggattacaggcgtaagccaccgcacctggcctaaaaatttctcagttttaatttctaatatggtagacattgataactataatccacataaataagctctttggaggctcattctaacagttttattgagaaataattcacatactgttataattcaccatttaagttatataattcaatgattattagtatattcacagagttgtgcaagcatcaccagaatcaattttaggacagttacaccacgcaaaaaagaaacctggctgggagtgctggctcatgcctgtaatcccagcactttgggacgtcggggcgggatcacttgagttcatgagtttgagaccagcctgggcaatatggtaaaaccctgtttctacaaaaaatacaaaaattagctgggcatggtggtgcatgcctatagtcccagctactcaggaggctgaggcaggaagatcacttggacccaggaggtagaggttacagtgagccgtgatcccgccaccgcactccagcctgggtgacagagtgagaccttgtctcaaaaaaagaaaaaaaaaaaagaaaccctatacccattagcagtcactcttcatttcccccaatctccccaggccttgacaaccactaatctactttatgtccctacagattttcctattttgtacatttcatattaatggaatcattcaatatgtggtttttcagtgtgtggttgcttttacttagtgttttttttttttgaaacagtgtctcgctctgtctccaaggctggagtacaatggcatgatcttggttcactgcaacctccgcctccccggttcaagcaattctcctgccttaccccccacccaagtagctggggttacaggcacccaccaccacatccagctaattttttgtatttttagtagaatcaggatttcgccatattggacaggctggtcttgaactcctcacctcaggtgatccacctgcctcggcctgccaaagtgctgggattacaggcgtgagccaccgcaaccggcctcacttagtgttttcaagattcatctacattgcagcatgtgtcagcccttcattcctttttatggctggataatattccattgtatgcctatcacattttattcatccattcatcagttttaaaggtgtgcagggatcctgagaccaagaagtttgagagcagctattctagggtttcttactctgggtacggacaaatgtactttgtgaaaacgcaccaagccatacacttacaggtatgtgtacctgggtttatatgcaaattacatttcagctttggcaaaacatttatttttctttttttaatatgaacactttatgaatttatgtgtcatctttgcacagaggccgtgctaattttctctgtctcattccaatttttatttatatgtgatgctgaagcaagcacagcacaacaaagcaattcttaagcctttattaattttgaaggccagttgtggtggctcacagctgtaatcccagcactttaggaggccagggtaagaggatctcttgagcccagaagttcaaagctgcagtgagccatgactcaccactgcactctagcctgggtgacagcaagactctatctcttaaaataaaatttaaaataaaataaaatgttattgagctcattattgagctgttccaggcagaatcctaagtgtctttgtggattctctggctgaatgcccacaatcacacctggaagtaagaattatcaatcctttaattttcaaggtgagaaagcaagagactcaaagaggtgcagcacctatccctgggtcacatagcatggaagtggcagagtggggtgtagactcaggctggtcatacccaaaccctaatcctccacctaccctcaactttttattttgaaaaaaatatccaggctgggtgtggtggctcatgcctgtaatcccagcactttgggaggctgagcagggaggatcacttgaggctaggagttccagaccggcctgggcaacatggcaaaaccttctctctacaaaaaatacaaaaaattagccaggcgtggtggcacgtgtctataatcccagctatgcaggaggctgaggtgggaggatcacctgagcctggggaagtcaaggctgcagtgagccatgatcgtaccattgcattccagcctgggtgacacagcaagaccctgtctccaaaaaaaaaaagacagaaaaaaatccaaacagaaaagttgcatgcataatatcgtgaacacctgcacacccgcacaccctccacctcgattcaacatggtaccattctgcttcgtctgctttatctgtgcgtgtacccacacacttactattattattttttgctgacccacttgcatgtagttgcacttaagctttgctaagaataaggatattcccctacacaatcacagtaccataatcacatgtaagaaaattaacaccaattccttaacatctgccatgcagtccatattcagattttccatttgtctccaaaaagtctttcatggctgttttttccaaactgatatgcaatcatggttcagatgctccatttgtcaaataatgtctctttcatctgtttttaatctggaatatccacccccacttgtttcaagacattgacttgttgagggaacctccacctaccctcaactttttattttattttttataacggcaaatgctacacatctaattccatctctgatgtacacagaccctcgctggcacttctattttcagctttctgctcctgctccaagttaggaactcccaatggcttaaatgttttctcagataatctaatatgtggctgggcacagtggctcatgctataatcccagcactttgggaggccaaggcgggtggatcatgaggtcaggagttgaagaccagcctggccaacatggtgaaaccgtctctactaaaaatacaaaaattaaccaagcgtggtggcatgtgcctataatcccagctacttgggaggctgaggcaggagaattgcttaaacctgggaggtggaggttgcagtgagccaagactgcgccattgtactccagcctgagcgacagagtgagactccatctcaaattaaaaaaaaaaaatctaatatgcttctcttaatcatgaccctgatagtggtttaggcctgttcaaagcacttcacaatgactctaagaggtgggtaaattatcaccacctccatcttccgcatgaggaaactggagctcagaggggtgaagtcacttgccccaggtcacacagcaaagccaggatctgaacccaggcagtgtggctcactcacctgactgcagacgacctcctggcatggacttcttagccatctcggccagtactcctagccccaagcccacagccagtcctggagtgcaagagaagaacatgagtgcccgtttgcctggggtgcccccagcctaacccactctcctttctcagtttttctccaagaatgcttgtctcccaactctttttattttttcttttgtttgagatggagtctcgctctgttgcccaggctggagtgtggtggcacaatcttggttcactgcaacctccacctcccgggttcaagcgattctcctgcctcagcctcccgagtagctgggattacaggcacgcgcccaccacgcctggctaaattttttttgtatttttagtagagacggggtttcaccgtgttggccaggctggtctcgaactcctgacctcaggtgatcctcccgcctcggcctcccaaagtgctgggattataggcatgagacaccgcactcagtcttgtctcccaattctttgtatctctgtttccaaatcagtctctgactctgttcctgcatggggctccctttaaggtgcagcttcctctttctttaaccctttctccatgtctctgtctccctcccagcagttctcagccctggctgcacattccagttaccagaggagctccggtaaatataatgcccagactccactccccaaattctgattaaattggcctgagacatgggaatttgtgttttgttaaaaagcacctcagataattctaatgtgcgatcaaggttaagaaggcccctcggccgggcacagtggctcatgcctatgatcccagcactttgggaggccgaggtgggcagatcatctgaagtcaggaggtcgagaccagcctggccaacgtggagaaaccctgtctctactaaaaatacaaaatcagccgggcgtggtggtgcatgcctgtaatcccagctactcaagaggctgaggcaggagaatcgcttgaacctgggagacagaggttgcggtgagccgagatcacaccattgcactccagcctgggaaacaagagagaaactccgtctcaaaaaaaaaaaaaaaaagaaggccccgaaacaattgtcctaggtccctctgtcttctggtgactcttctttctctgtccacacctcctcttctttatgcttatctctttttctttttagagacagggtctcactctgtcgcccaggctggagtgcaatggcaccatcataactcactgcagcctcaaattcctgagctcaagtgatcctcttccctcagcctcctgagtagctgagaccacacgtgcataccaccacgctcacctaattttttattgagacagggtctcactatgttgcccaggctggtctggaactccagaactcacgtgatcctcctgcctcggcctcccaaagtgctgggattacaggtgtgggctaccaggcttgacctctccctttctaagccagttatttctgcatctcttgccttctccccatttgtcctatctatctagatctctttctctcttttcctccctttgatgttatctctccatctctgatttcctctgctacctctcctcttgtccctaaagacgtctcgggtctctctccttcttttgttgtcaccatctctgtcaattaccatatctggcattttctgtgtcctttctacatgtcaggggctgttcccagtgccttctgtgcacttactaatttttgtccttacatgaggtctccaccttcataaaatgtattttacagaaaacttccattttacagaaaaggaaattgaggttcagagaagtgaagtaatttgcccagagtcacgtcacaaagcgaggacacagcagagtcatcccttaatccctgggcagcctggtgccaaagctctaaccagtgcctttgactgcctctgcagactgtgaatctatctatgtatatatatatagattttttttttttgagacagagtttcactcttgctgcccaggctggagtgcaatggtgcgatcttggttcactgcaatctctacctcctgggtacaagcaagtgaatctatatttttaaaacttgaaccaggcggggcatggtagctcacgcctataatcccagcactttgggaggctgaggcaggtggatcacgaggtgaggagttcgagatcagcttgaccaacatggtgaaaccccatctctactaaaaatacagaaattagcggtcaggcacagtggctcacgtctgtaatcctggcactttaggaggccaaggcaggcggatcatgaggtcaggagatcgagaccatcctggccaacatggtgaaaccctgtctccctaagaaaaaaaaaaaaaaaaacaacaaaaaatagttggacgtggtggcgtgcatctgtaacccccgctactcaggaggctgaggcaggagaattgcttgaacctgggaggtggaggttgcagtgagctgagatcatgccactgcactccagtctgggtgacagagcaagactccatctcaaaaacaaaaacaaaaacaaaacttgaaccatgatcagcaataagaagacatttacgttataaccagtgcagaccaaggtatatgtttcacgaaacaagactttcccttgctctacaggtgcatgcatattttctgtcctattcttctttttaaatgctggttgcaacccactaaattatttcatgtgacttgcacgacatccggctgtgtaacccaaagtcaagtgacctcccctttgttagcctcaatgtcctcatgtacggaatagaacactcattgattcagtgcagcatgtgtgtatatgggacactgcacattacaggttctcagtaaatgttattatggccaggagcagtggctcatgcctagaaccccagcacttcgggaggccgaggcaggaggatcgcttgagaccaacttgagcaacatagggagaccctatctctacaaaaaaatattttttaaattacccaggccaggcacagtggcttcccgggcaccgtggctcacatctgtaaatcccaacactttgggaggccgaagtgggtggatcacttgaggtcaggagttcaagaccagcctggccaacatggtgaaattctgtctctactaaaagtacaaaaattagccaggcggccaggtgtggtggctcacgcctgtaatcccagcacttgggaaggctgaggcaggcggatcacaaagcgaggacatagcagagtcatcccttgaaccctgggcagcctggtgccaaagctctaccactgcctgacaaacatggtgaaaccccgtctctactaaaaatacaaaaattagccgggtgtggtggatgcatgcctgtaatcccagctacttgggagtgtgaggcaggagaatcggcttgaacccgggaggtggaggttgcagtgagccaagatggcgccactgcactccagcctgggtgacagagggatactccgtctcaaaaaaaaaaaaaaaaaaatagccaggcatagtggcaggcacctgtaatcccagctactcaggaggctgtggcaggagaattgcttgaatgtaggaggcagaggttgcaatgagccaagacggtgccactgcactccagcctgggcaacagagcgagactctgtctcaaaggaaaaaaaaaaaaaaattacccgggcggatggtgcacaactgtggtcccagctactcaggaggctgaggcgagagaattgcttgagcccagaagtttgaggctgcagtgatcgcgccactgcactccagcctgggtgacaaagtgagactctgtctcaaaaaaaaaaaaaaaaattatcatttatgttcctcttgatttctcttggccttgcaggcccttgcctttgtctctctttttttgtctctctctctttcctgtctctgattcatcctctcccttgcttcaatctgtaaatgttgccctttctaattgaggtcacaccaagatcccccaaagtcacacctacccccaaagttggccaagcggctgatgcgggaggcaggcaccttgcgttctcgagagcggtcactcagctgggaaatggggacaaggtctgagggtgggaaagtgggcatcgtggccagagagtgcagccactctgccaccacacccttccccaagctcagggggccagcagtattcgtggggtgttgctgggtgggtgggggtaatgatacctggggccggggtgtcttcctgggacgggcctcccgtgccctgcgaatgtcctcctcacccaggcctctcccaggcccatcctggtaaaacttttgggcccaagaacctccacatggtccctgcaagagatatactttgataaggaggggaggacatgggatcacctggcccttctggacccctcctgtccttttaccctctgaagtctccctcagcctcttccccttggggacctgctccctagcctcttacccagcggtggggcccaggccccagggccccacaaggccaaccaacagtctggcccagctgtccaccggtcccccgaagtaggccccccaccttcagccacattgcctggaggagaagaggagggattattcaggtgggagttgcctggcttcttggccaggcccccatgttcctctgtgtccaccctctctcattcccacccactaaatacttcctccactattaatagattcccacagtttctcctggttctcccctggttgctaggaacccttagctcttcctgcaggcctcccaaaacccactttcccccctctcccaaccctcgccgttgctagggtccgcccccgttactagacacccacagttcctcaggggctacctctccacttctctgaaacccccctcgttgctatgcaccgccttcccctctgttggccctgtaggcacccgcaattcctccggtgtccccccgcccgatccccaccccgttactgagcacctccccccgttgctaagcacccactgctctcagcctccgagtccgcaacctgctggggccctcctgtgcttccgggagcctctgtatgcccgcccccttcggccagccaaccaatggcgcggcagcaactcgctgactggcacggagtttctccatctgtgcgcgggggaaggggccagagagaggccctagcgatggagcctggaattaaggtgggcatcgggggcccatctggggggattgctgaggcatcgcgggagcgcaagcgtgccctgtctgcccttccactcacctcaaagtgccttgtacgcgacagacccttctgttccctacactttcttgcaaagtctcccttacaaacctgacgcctctcttctgcacgaatatcttgccaaccccccttcttatttttacccctggcaggctcaacacaattcctttgaatacacattcttcccagcaaacatttcccttgcacatggagcccctccactttgcacaaacacccacattctggcacccaacctccttccttgtgcatataaatcccttgccttgcttgccaactgtttcccgctcacgccttcaactctctcgtgtacataccctcccccccacttgcacactgagtctctgttccctcacacaccctgttctctccgctccccgcccacctgcggaatggtgcgactgggagccctctgcttcctgtaggcctgacatttcccagaacacactcagccaagattcccagtgctgagtcaggaaccttggcatcccagaaaaccacctcccatcctatctttctttctctgtgtgtgtgtgtgtgtgtgtgtgtgcgcgcgcgcgttggaaagcctgctctccccacaacaggctgactcacccacttctcacagtctggaatagaggcatctggttggggcatctttcccaccactgggagagggagggatgccgatgcttcccccacagaaccccgctgccccaacccctcttgtcgggaatcaggcctctttgtctctattcccgtactgtgtgacctcagaaaagccctcatccctctctggacttctccttcccaacctgtatgaggtactaggtgggctcctgccttcgatttctctcattggtctgtgaaaaggtgactcattttagagccttggacctgtgcccaatgtctcccccagttgactcaatttcaagacaaaccacctcctctcccccttcagaggtgaacggctgactcaggagctgggtggcagctcagccttgggggcaaaatatatcaaaatcacctaggacaataaataatcctactgggctacaactgacagtgccaggcccggggccacatgcttcacatacatgagatcatgtcagatagccccgtagctgcaagcaaagggtagaggccggcagatctctgagcctctgttctcacgtgtaaaatggagccctggcatccctattgttcagccttggagaatgaactgaaatcgtgtcagggtctgatacagaacaagtactctatacatgacagagatttcttacggaaaatccaatggagcgtaagtcatttgctcagggtagcggagctgggaactggcagaggaaggtctcgaacccacacccgtgggaactccaagtctttgaaagtccgtcccgcccctgcgtgaagaatgggcgcgcggctggactcgtcaccgggggttttagggaaggttgaacttgtcattcaaccgtgtagggagggtggtttagtcacaggggtggagggagggggcccactggcgcgccaccaggaccccggtgccctagatgcatacctggccgcgcttcggatgctcagaggcaaaccccctccccttgggaggcggagtcggacaaggctccgtctcgttccgttccgtcacgtccggtggggcggggcttcggggctttcccctttgaggggagtatcggttaacccttgcgcggcggggcgggctggagctccgcgggccggaacccgggagtcggggctccccagccacacccctcgcgggatttaaagggataggaggggccgggtcggccgaagcccgaaccgaaggagcgggcatgaggcgctgcccgtgccgtgggagcctgaacgaggcggaggccggggcgctgcccgcggcggcccgcatgggactggaggcgccgcgaggagggcggcggcggcagccgggacagcagcgacctgggcccggcgcaggggccccggcggggcggccggaggggggcgggccctgggcccggacagaggggtccagcctccacagcgagcctgagagggccggcctcgggcctgcgccggggacagagagtccgcaggcagaattctggacagacggacagactgagcccgcggcagctggccttggagtagagaccgagaggcccaagcaaaagacggagccagacaggtccagcctccggacgcatctagaatggagctggtcagagctggagacgacttgtctttggacggagaccgggacagatggcctttggactgatccgcacaggtccgacctccagtttcagcccgaggaggccagcccctggacacagccaggggttcatgggccctggacagagctggaaacgcatgggtcacagactcagccagagagggtcaagtcctgggctgataacctctggacccaccagaacagttccagcctccagactcacccagaaggagcctgtccctcaaaagagccaagtgctgatggctcctggaaagaattgtatactgatggctccaggacacaacaggatattgaaggtccctggacagagccatatactgatggctcccagaaaaaacaggatactgaagcagccaggaaacagcctggcactggtggtttccaaatacaacaggatactgatggctcctggacacaacctagcactgacggttcccagacagcacctgggacagactgcctcttgggagagcctgaggatggcccattagaggaaccagagcctggagaattgctgactcacctgtactctcacctgaagtgtagccccctgtgccctgtgccccgcctcatcattacccctgagacccctgagcctgaggcccagccagtgggacccccctcccgggttgaggggggcagcggcggcttctcctctgcctcttctttcgacgagtctgaggatgacgtggtggccgggggcggaggtgccagcgatcccgaggacaggtctggggtgagtgggacccatcctgcccttgagccacatcacgcaaaactccttattcctccgcctttgcttaggaagttctctacccatttactgttagttgcccaccagcaatttcatctccgggaacctcttccatccactgacctcctccctctgacagccaggttactaattcactccaggcaccccttcttaattctctcctttccccttcctgcactgtctccactcctgggggtctacaatggagggccactgaccgactttatggggcccaggaacccctgaaattgtaggactaaggagcctgccgtgcgtgttcacatacattgttgcagcgacggtctgacatctgtctaatcctctggaatctgaagaacactgggatagggagacaggcagggtgcactttctaccccacatttcttaatctggggcctgtggctgtcttaaagggtcacaaaccttcaccaccccttagcctatttgtatgtacatttttctagggagcaaagcgattcccccagcttgcatcagattcgcaaaagggctgtgtgaccccaaacgccaaaggatccttggcctagccgggagcccacctggtctcccctgtgacccctacatccccaaggagccccctgaacactcttgtttactttgtccaccgtcacccccagcttgggtgtggctctcccctggtgaatcaggaggaccggccgggcaggttcagggagggcgggacagagcagaggccggtgtgaaactggagagcctcacgtggggctgggagccgtggggctgggagccgagtccggagtccatcagcttgccagcttgcctgctgaggcctctttttgctctggggcccctggctggagtctgccctgagccccgcttcaccccacatgccttccttggggacgtgttcacacatgtggccctagctgtgagagacagacctgccttgacgtgcctgtgcctgtgtgcaggggctgaccctcctgggccccattgcttttttctctctgcctgccctctcacttccttggcatctcagaacagctgagctggaagtgggtgaataataataataataataataataataacaacaacaattagcactcactcatgtttagccctgtgctaagtgctgtgcttttattaactcactcactcctcgcagcaaccctaatgaggtagatactttttttttttttctgagacagtctcgctctgttgcccaggctggagtgcagtggtgccatctcggctcactgcagccttcacttcctgggttcaagtgattgtcctgcctcagcctcctgagtaggtgagacaccaggtgcccgccaccacacctggctcatttctgtattttgagtagagacagggtttcatcatgttggccaggctgacctcaagactcctgacctcaagtgatccacccgcctcagcctcccgaagtgctggaattaccagcatgagccactgcacccggctgaggtagatactattattatccccttttacagatgaggaaactgaggcacagagaattcaagtcacttgtccaaggatatcaccttgcaagatgcagatcaaataattctaatccttactgtacaccagacactgttttaagttttttcttcttttttttttttagtcttttttgtcaatttttttctgtcattttttatcttaaaatagcatctatcttaattaagggctttgtatgtgttaactctgagtccttatgacagcactgagattgtctccattcaacagatgggggccaggctcagtggcccaagcctgtaatcccagcactttgggaggccgaggcagggggatcacctaaggtcaggagttcaagaccagcctggccaacatgatgaaaccctgtctctactaaaatacaaaaattagccaggcgtgtgatggcgggcacctgtaatcccagctacttgggaggctgaggcaggagaatcgcttgaatgtgggaggcggaggttgcagtgagccgagattgcaccactgcactccagcctgggtgacagagcaagactcggtctcaaaaaaaaaaaaacaaaaacaacagatggggaagctgaggcatagagaggtgagagattctttgcttagggttggccacgtcaagtcagagccaggattctaactcagttctggctgagtttcaaacccatcgtttaaaccctccagtgtgtagatctgttcctaacttgtctccctcccaggcctccctatctccagtttttttctcatccttccagaatctgcctccatggcccagctttgtctgctcaggtgccctctctgagagcagtcaggggctcagtgctgcgtttggagcctccggtctccaggcgccttcccagaatcgacttccttccctcctcctgaccccctctgctgtaactaggcctgagtacacactgttccttgaacactcttctgttgtcctgcctccacgcattgctcaagccgttcccactgcccagcatacccgtgcttccttctcccttaattttgaaatcctctctgaccgtaaaggctcagcttcctttctccctttcctccttccttccttctcaccatcctgtcctttttttaaaattgtgttctctttctttttctttttttggtagggatggggtcttgctatcttgcccaggctggtcttgaactcctgggctcaagtgatcctcctgcctcagcctcccaaagtgctgggattacaggtgtgctatcctattcttctaatgagacaaaaatcactccctaagcccctaccatgtatttggtcctatgttagctttgctggggaaacagcagtgaccaagacactcatggagctcccaggcccatgaaagagacagaccaatcagccgacagtcacagttcagagtggtcaggaggatgtgtgagcccagaaggaggtcactggccagacacagggtatcagagagggcttcctggaggaaggggcatataagctgagaccattgtgaaaaaaatccgagaaagcccagataaaccattggatctaactttttttttttttttggagggcgtgcagtgcggagtttcactctgttgcctaggctgaagtgcagtggcgagatctcagttcactgcaacctcagcctcccgggttcaagtgattctcctgcctcggcctcccaagtagctgggactacaagcgcacgccaccacgcccggctaatttttttttttagtagagagtgggtttcactatgttggccaggctggttctgagctcctgacctcaggtgatccacccacctcagcctcccaaagtgctgggattacaagcatgagccactgtgcccggcccggatctaacataaaacagagagagaagatattatgtttatgattaaggggccaggtgtggtggcttatgcctataatcccagcactttgggaggctgaggtgggaggattgcctgagcccagaaatttgagaccaccctgggcaacacagtgagacctcatctctactaaaaggaaaaaagaactagctagatgtggtggtatatgcctgttttccagctattcgggaggctaaagcaggaggattgcttgagcctgggaggcagaggttgcagtgagccaagatcacacctctgcactccagcctgggcaacagagtgagatcctgtctcaaaaaaaaaaaaaattactattaaaaaaatcccaactaacattcttcagtccaagcatcaaagaatgtttctggagcgtctgtcgtgtgccaggccatgtgctggggacataatcataaccatgacaaccccagtctctgccatcatgagcttgtagtccagcaagtgtgtaggggataagccccaaggcactgggatccaaccagagctagaaccccagcgctgtgtggggtgatgttaggtaagtggcttcacctctcagaccctctgtttcctcctttgtaacaccgagacgataatctccctggggttgtgtttttagaaattaagtaggttgtggcacactgagtctttgtcattgtgtctggaatgttccaggtacttagtaaacaggaacctttattaatcatctcttccccagggccggcagctggattggggagggactagcccttcccgaaccctgactccctcttaaccttcccactgtcaaagagatgtctgtgaggtagctgggctctgagggggcgggagagatgaacacgtccctgatcctggggtaggagagggatgagccatacagtttccttctctggaggtgacccactcttccagtacttgctaagggacttgacatgcttgtggggtgcagaatgtattcctgtgtgtgtatgtgttcatgaatcagacactaaccctgtctcaaaaagctcatagtttagtcggcatgatacaacgaaaaccctgttttctatttgcttctctggttttctttctttctttcttatttttttgagtcagggtctcactgggtcacccagtctggagtgcagtggcacaatcactgctcactgcagcctcaccctccagggctcaagtgatcctcccaccttagcctcctgagtagctgggactacaggtgcacaccaccacgcctagctgattcttaattttttatttatttatttatttattttttgagatggagtctcgctttctcgcccaggctggagtgcagtggcacaatctcggctcactgcaacctctgcctctcaggttcacaccattctcctgcctcagcctcccgagtagctgggactgcaggtgcccgccaccatgcccagctaattttttgtatttttagtagagacggggtttcactgtgttagccaggatggtctcaatctcctgacctcatgatccgcctgcctcagcctcccaaagtgctgggattacaggcatgagccactgcgcccggcctaatttttaaacattttgtagagatggggtcttgctatattgcccaggctggcctcaatctcctgggctcagtggtcctcccaccttggtttcccagtgttgagattacaggcgtaagccatcgtacccgctgcttttgtggtttcctttgttctctctctctttgtctacttctgttcatctttttcttttcatcattactttgtatctctctcttttagtcttcctatatttgttttgttttttttttgtttttgagacagtcttgctctgtcgcccaggctggagtgcagtggcgtgttcttggctcaccgcaacctctgcctctcgggttcaaacgactctcatgcctcagcctcctgagtagctgggactacaggtgtgtatcaccacgcccggctaatttttgtatttttagtagaggtggggtttcaccatgttggccaggctggtctcaaactcctgacctcaggtgatctgcccacctcaccctcccgaaatgctgggattacaggtgagagccaccacacccggcctatttctttatcttttagcaattctatacctgtctctgtattagtcagggtaggtcaggattttctgcagtaacaaagagcccccaaatcttagtgacttagaacaacaaaggattattttgctttatactacacattggtcacaggtcagtggtgggtcaagggctctgcttcatgctgtcttcactcagggtcccaggctgagggagcttccatttcttttcttactccctcccccactggggaacaaacgtggcgagtcataaactggctcttaaggctcctacccgaaagtgacacttctgctcccagttaattggccaaagcaaggccaggtgtggtggctcacacctataatcccagcactttggagaccaagatgggaggattgcatgaggccaggagttcaagaccagcctgggcaacatagcaagatgccataaagtagctgggcatggtggcacacgcctgtagtcctgactactcaggaggctgggatgggagcatcgcttgagtccaggagttggaggttgcagtgagctatgatggtgccattgtactgcagtctggctgacggagtgagacccggtctctaaaacaaaacaaaacaaacaaaaaacaacaatccccagttcaggagggcagggacatccctgaactaatagctggagctatgtggtgaacagctgtcatgactgccacggtctctgtcgctgtctctgtctctctgtcatcactatgtcatctgcctctctgtttctgtctctctctgtggtccatctccatccatcaactcctgcctctataattttccccaaaaggtctgacctggtttagggacagagatggggcttccaatatttgacactggtccctacataatggcttatgcctgaaaccccaacactttgggagactgaggcaggtggactgcttgaggtcaggagctcaagaccagcttgggcaacatggtgaaaccccatctctactaaaaatacaaaaattagccagacatggtggcatgcacctgtagtcccagctactcggagggtgaggcaggaggatcacttgaacctcggaggcagaggttgcagtgagccaagatcataccactgcactctggactaggtgacagagaccctgtctcaattaaaaaaaaaaaaaaaagaagatttggaactgtgtatgtgcagtggaagatgtaagggttgtttgttaaaccacaacaaaacctagcctgtccttactcataagatccttactcataagagaaggacagagagacatagatacaaagaaagtgatgacagaggggagaaatgggcaaagaatttcccctatcttgctgagggctttgggcgatcatgtgattgccgaactaagtggggctcactcaccccaacaagcctgggaacagtgatcccaggaagcccctggccctggaaagactgcaggtcccctttcgttccctctcatggcagcacctctagcccctgccttccctggccttggccctgaccccaccctgctctgctccctacagagcaaaccctggaagaagctgaagacagttctgaagtattcaccctttgtggtctccttccgaaaacactacccttgggtccagctttctggacatgctggtaagtggggtggtggtggacagagctgggcagagtctcctgggccagggaaagggatgttctctgtagtttagttcccccacctagtgatgggcttggtgacagtccccaccttatgggactgttatgggcagtgcttaggcctgggcctggcccttcgtatgtcagggcagagggctattccttgttattctggtggagagagggagcttcagacggacctgagctcaaaccctagttctgctctgtattcatgggtgaccttgggcaagttactttcctttactgggcctcagctgccccatctgttaatgtggagataaccttacctcccttgcaggattaaccgaagagttaaaatcaaaatgtatataaagattgaggccaggcacggtggcttacatctgtaatcctagcactttggcaggccaaaacaggaggatcgcttgagcccaggagtttaagaccaacctaggcaatatagtgagaccccgtttcaattaaaaaaaaaaaaagattggctgggcgcagtggctcatgcctgtaatcccaggcctttgggaggccgaggtgggtggatcccctgaggtcaggagtttaagaccagcctggccaatgtggtgaaatcccttctctactaaaaataaaaaaattagccaggcatggtggcgggtgcctgtaatcccagctactcaggaggctgaggcaggagaatcacttgaacccgggaggtgaaggttgcagtgagccaagatcgcagcattgcactgcagcctgagtgacaagagcgaaacttcgtctcaaaaaaaaaaaagactgaggcacaggactcttatgaatggaaatgatcactccttgccttctccgcgtgttcagagcatagcaggccttcagtaagtagcagctgtaacattttgcccccaggaccttgcaaatgcctgcagcacagagtagcacaatggtggcatatagtgctcttttgaaaggctaggccataaatatcttaggttttgtgggcctatggtctctgtcgcaactactcagctctgctgttacagcaggaaagctgccatagacactatgtaaacatatgagtgtgtctgtattccaataaaactttatttgcaaaaaccagcagcaggctggactagaaaagtgtgcccattggcaaatggcgacagtaagagaatttgtctgccttgtttgtctttagatctgacctggaaataaggcaagaccccctaaaactgtgtcaacacaagtttgccatctatatgtgttctggttactattgctgagtttattaaaaaaacaaaactcagcagagcacagcggctcatggctgtagttctagcactttgggaggctgaggtgggaggatcacttgagcccaggagtttgagaccagcctgggtgacatagtgagaccttgtctctacgaaaaataaagaaattaactgggcatggtggcatgcacctgtggtcccaggtacttgggagctgagggtgggaagatcacttgagtccaggaggtctgggatgcagtgagccttgatcgcaccacagtactctagcctagatgaccgagtgagaccctgtctcaaaaataaatacgccaggggcggtggctcacgcctgtaatcccagcactttgggaggccaaggcaggcggatcacgaggtcaggagatcgagaccatcctggctaacaaagtgaaaccccgtctctactaaaaatacaaaaaattagccgggcatggtggcgggcgcctgtaatcccagctactcgggaggctgaggcaggagaatggcgtgaacccgggaggcggaggttgcagtgagccgagatagcaccactgcactccagcctgggtgacagagcgagactctgtctcaaaaataaataaataaataaaataaaataaaataaatacaaaacaaaacccaaaaaccccaagacttagtgatggttaaaaaaaataacttagtagaataaaacaatcattttattgacctcagaagttttgggtcagcaattcagacaggatggcagggatggcttttcactgctccacaatatctgggaactcagctagggacacctggaggctacaagctgaaaacttttggaggttcattcacttgcatgcgtggtgattgatgctggctattggctggggtctcagatggggctgccacgtggctttgccatgtagctccttgagcttcctcacagtatggtggcctcagagtactcagatttctaacaagacagctcaggactaccttgaaggtaagtgtctctgccaacagaagtcatgcagtagcacttctgccataaaccgtcccatatttaatgggagagcaatctattttttatttattcatttaattttgagataggatctcagtgttgcccaggcgggagtgcagtggtgcagtcatagctcattgcagccttgaactcctggccttaaaggatcctcctgcctcaacctcccagagtgttgggattataggcatgagccactgcacccagcttgggagagcaattaagagtatactccttgatgggaaaattctataagagcaggatcaggaatgggtctgagacccactgtgatcaggagatactgttgtagccaactttggaaaatgtaatccaggctggacataatcacagcactttgggaggctgagatgggaggatcatttgaggccaggagttcgagaccagcctgggcatcatagcaacactaccccaacccgacattacacattttttttttttagattagctgggagtggtgggggtgcacacctgtagtcctagctactctggtggttgaggtgggaggatcacctgaatctaggagcttgaggctgcagtgagctatgattataccactcaatttcagcctgggtgacagagtgagatcctctcaaataaaaaaagaaaaagaaaatataatctgaaaatacagctggccactagaagaaattagaattattataatctatacactctgcgtggtccaggaaatgcttgatctataaattaacttaaataggccctggttgttagcaccttgggattccaggcagcagcaaaagcaaatccactctggagagtgacgctctggacttagatctcacagaattcccacagatgatcacatgtgtacaatcaaaaattacaaaacaaattaggatatcctgtgctgggaacaggacttggtttaagcaacagagaacagaaattcgccctttccttaccaacccctgggcaagacttttaagagttgcggcctgggcacagtggctcacgcttgtaatcccagcactttgggaggccaaggcgggtggatcatctgaggtcaggagttcaagaccagcctgaccaacaaggtgaaaccctgtctctactaaaaatacaaaaattagctgggcttggtggtgtgcacctgtaatcccagctacttgagaggctgaagcaggagaattgcttgaacccgggaggcggaggttgcagtgagccgagatcgcaccattgcactccagcctgggcaacagagtgggactccatctcaaaaaaataaaataaaatcttaaaatcattaaaaaaaaagttgcagggagagaaagcattgcaggagggtcgggcaagataatggtggccaggatctgatgcaaacaggcagctgcggagaggttctcttcctgatccagttcctgatcctctcatttattctaccacctttagggaacttccaggcaggagaggatggtcggattctgaaacgtttctgtcagtgtgagcagcgcagcctggagcagctgatgaaagacccgctgcgacctttcgtgcctgcctactatggcatggtgctgcaggatggccagaccttcaaccagatggaagacctcctggctgactttgagggcccctccattatggactgcaagatgggcagcaggtggggctggggcagccctggggcagggatggagggcagggggtgggcattattgaaaatattggcctggccaggcgcggtggctcacgcctgtaatcccagcactttgggaggccgaggctggtggatcacctgaggttgggagttccaaaccagcctgaccaacgtggtgaagccctgtctctactaaaaatacaaaaaattagccaggcgtggtggtgcatgcctgtaatcccagctactcaggaggctgaggcaggagaattgcctgaacctgggaggtggaggttgcggtgagctgagattgagccattgcactccagcctgggcaacaagagtggaaccctgtctcaaaaaaaagaaaagaaaagaaaagaaaatagcgccaagtccattttcaaagtggttgtaccagtgtaccttcccaccaccatgtatgagtgttacagtcgtcccagattctcaccggtgcttggtactgtctgttttattaacttaaaccattccgatgggtttggaataggatccctttttttcctctttgagacagagtcttactctatcgcccaggctggagtgcagtggcacgatctcggctcactgcaatctccacctcctgggtttaagtgattctcctgtgtaaaccaagtagctaggattacaagcgcccgccaccacacctggctaatttttgtattattagtagagatggggtttcaccatgttggccaggctggtctggaactcctgacctcagatgatccacccactacggcctcccagaatgctgggattataggcgtgagccaccacgcctagtctgcaagaagtgaactctaattgctttctccagctgggttcagtggctctcacctataatcccaacactctgggaggctgtatacacacacacacacagacacacacacacagacacacacatagtcttcaactctcccatttcattcttttagtagtcttttgataaacatatttttataaacaaaatagtatttgtattattactcttttttgagacagagtctcattctgttgccccgactgaagtgaaggagcatgattatagctcacagtagccttgaactcctgggctcaggcggtcctttcaccttagcctccagagtagctgggactataggtatgtcccaccaggcttggctatattttattttttatagagacatggtctccctgtactgcccaggctggtcttgaacccctgagctcaagtgatcctccttcttctgccccctgagtagctgggactgtaggtgcatactatcacacctagctaattttcaattttttgtagagacaggctatttaggaaaagctctgaatccatgcttttccgttctcacaccactgccacaacaatgagagcacagaagacttctgtgaccaaatgtttaggttttttaaccccacacaccaagcaacggacaccagctggatgtcctccaattcaagtctaaaaccatctacctggaaatagtgtcagatcccacaggttggggactcagtccccaagactgccccccagttacagacaccagtcataagttcaggccttcgaaacttctgactgattggctccaagttggagttcccatgtcctcctctttgggttcaaataatttgctggagtggctcaaataactcagggggatacttacttacctttactggtttaatataaagcaggggtccccaacccctgggccgcagaccagtaccggtccatggcctcttaggaacggggccccaccgtaggaggccagcgagcattaccgcttgaactccacctcctgtcagactagcgggcaggagattctctggagggcgaaccctattgtgaactgtgcctgcaagggatctagattgtatgctcctatgagaatctaattgatacttgatgacctgaggtagaagagtttcatccagaaaccatccccctcacagtctgtgggaaaaattatcttctacaaaaccagtccctggttgggtactgctacaaaggatactgcaaaagatgaagagaagtgtagggcaaggtatgggggaaggggtacggagcttccatgccctccctgggcgcaccacgctccaggaacctccacagggtcagctatctggaagctcctgagcccaatccttggtttttttttttttttttttttttttgagagggagtcttgctctgtgtcccaggctggaatgcagtggcctgatccgcccgccttggcctcccaaagtgctgggattagagatgtgagccaccatgcccagcctaatccttgggtttttatggaggctttaagacaccagcattccttcccccagagtatagggtgggatcctctctggggagggtcctaagacccacagtcaggaaggtggggaaagattagagtcctgccttggggcaggtgaaaggagggcaggaggaggtcagagattttggtttcttgagccagcttctgagatctaacacacctaaaattataacaaaagattgtggctgggcgcagtggctcatgcctgtaatcccagcactttgggaggccaaggcaggcagatcacttgaggccagttcaagacaagcctgggcaacatggtgaaactctgactctactaaaaaaatacaaaaatgtgccaggtgtggtggtatactcttgtaattccagctactcaggaggctgaggtgggaggatcacttgaacccgggaggtcctagaagttgcagtgagccaagatcacgccactgtactccagccttggtgacagagcaagactccatctcaaaaaaaaaaaaaaaaaaaggctgggcgtagtggctcacacttgtaaatgggaggcagaggcaggccgatcacgaggtcaggagttcgagaccagcctggccaacatcgtgaaaccccatctctactaaaaatacaaaaaattatccgggtgtggtggcgggcacctgtaatcccagctactcaggaggctgaggcaggagaatcgcttgaatccgcgaggcagaggttgcagtgacccgagatcaccaccattccattgcactctagctcgggcgggcgtcaaaaaaaaaaaaaaaaaaaaaaaaaagattgtaacaagggctatgagagttaccagccaggaaccatagagggaaaccaatatacattgtaactaacaccgggtctcgctatgttgccgaggctgatcttgactttctgggctcaagtgaacctcctgccttggcctcccaaagtgttggggttacagatgtgaacactgcacctggccctacctttaatttagtccagtttgtcagtttttttcattgtgtttaagaaatcttttcctactccaagatcatgcaaatattatgttttcttctacaagctttattgttttagctttcatatttagacctacattccagatagaattaatgtttctgtgtgtgggaggtagggatcaggttcatttttttccatatggattgttgacccagcactgtttattgaaaagacttccctttccccactgaattgcagtgcctagtcataactgcacaactctacgtgtgtgtctttgtggactctgttgtattctgtgcattgatttgcccattcttgtgccacactgtggctttatgtcttaatatcaggaagggtaagccctccagtttgttctacataatttcctttgtcacatcctctgtgtgctcaggacctatctggaagaggagctagtgaaggcacgggaacgtccccgtccccggaaggacatgtatgagaagatggtggctgtggaccctggggcccctacccctgaggagcatgcccagggtgcagtcaccaagccccgctacatgcagtggagggaaaccatgagctccacctctaccctgggcttccggatcgagggcatcaaggtgaggaccaggaaccgcctggcctgtcccgggaaggcctatagccagatcccaggcagggcttcttggggaaagccacgagagagtgcaggagacggcgtgggatgaaaggctggggctggggaagcccagcctctttcttacctccatacccatcagagcccaggttctgtttatccgtctgcctgagcatctcctgagtccatcttttcctcctctccttctccatggtcatgtcctagtccagccccatcttctcctgcctctgtctccctgggcatcctggccctggtctccctgcagcacaagagctggagggatccctttcccccagctatggagaaggtttctctttttctttcttgcattcctttttctttgtcaaattaaatataattgtatgaataggccaggcatggtggctcatgcctgtaatcccagcactttgggaggccaaggcaggaggattgcttgaggccaggagtagttcaggactaagctggccaacatagggagcttctgtttctagaagaaaaaaaaattgtttgaatagctagtcacacagttcaaaagtcaaaataatattataaaagggtaaatgtggagaaatttctctcccaactctgctgctgtccttcccattctgtccctctatccctctacctgctcatggggaaccactttgtcagtttcttgtatttcttcctgttagtgccagtaaatggagttgtagatccgaaatgttcctcagtacgcatactgattggtactttgctattttcacttaatagtaacaccctgcagagttttccacatcattccatagaaagcttcctctttctgtttttttttcccccacagctatgtaacactgtttttgtgactgtgcctggtcccctatagacagacacctgggttgtttccattttttgccattagcaggcctcaaagaatcactttgtatataagacattttgcaagtatgcagattgagctgtagatttccagaagcaggactcctgggtcaaagctt

SEQ ID NO: 8 - protein, 85 aa, Mus musculus
GFLPTRRPEPRPDPGPQPEPRPRPEPRPRPESRPRPEPRPRPEPRPQPESQPRPESRPRPESQPWPEFPLPSIPAWTGPEIPESG

SEQ ID NO: 9 - protein, 73 aa, Mus musculus
GFLPTRRPEPRPDPGPQPEPRPRPEPRPRPESRPRPEPRPRPEPRPQPESQPWPEFPLPSIPAWTGPEIPESG

SEQ ID NO: 10 - protein, 47 aa, Rattus norvegicus
GFLPTRRPEPRPDPGPQPELRPEPQPRPEFPLPSIPAWTGPEIPESG

SEQ ID NO: 11 - protein, 37 aa, Canis lupus
GFLPTHRPEPPPEPRPGPELPLPSIPAWSGPEISESG

SEQ ID NO: 12 - protein, 37 aa, Bos taurus
GFLPTHRPEPRPEPRPGPELPLPSIPAWTGPGIPESG

SEQ ID NO: 13 - protein, 41 aa, Homo sapiens
GFLPTHRLEPRPEPRPDPRPGPELPLPSIPAWTGPEIPESG

(19) United States
(12) Patent Application Publication
(10) Pub. No.: US 2026/0193346 A1
(43) Pub. Date: Jul. 9, 2026

(54) MITIGATING TISSUE DAMAGE AND FIBROSIS VIA LATENT TRANSFORMING GROWTH FACTOR BETA BINDING PROTEIN (LTBP4)

(71) Applicant: IKAIKA THERAPEUTICS, LLC, Oak Park, IL (US)

(72) Inventors: Elizabeth McNally, Oak Park, IL (US); Ahlke Heydemann, Oak Park, IL (US); Ermelinda Ceco, Chicago, IL (US)

(21) Appl. No.: 19/559,080

(22) Filed: Mar. 6, 2026

Related U.S. Application Data
(63) Continuation of application No. 19/281,249, filed on Jul. 25, 2025, now abandoned.
Application No. 19/281,249 is a continuation of application No. 18/980,341, filed on Dec. 13, 2024, now abandoned.
Application No. 18/980,341 is a continuation of application No. 18/646,068, filed on Apr. 25, 2024, now abandoned.
Application No. 18/646,068 is a continuation of application No. 17/811,233, filed on Jul. 7, 2022, now abandoned.
Application No. 17/811,233 is a continuation of application No. 17/536,768, filed on Nov. 29, 2021, now abandoned.
Application No. 17/536,768 is a continuation of application No. 17/219,845, filed on Mar. 31, 2021, now abandoned.
Application No. 17/219,845 is a continuation of application No. 16/994,225, filed on Aug. 14, 2020, now abandoned.
Application No. 16/994,225 is a continuation of application No. 16/716,382, filed on Dec. 16, 2019, now abandoned.
Application No. 16/716,382 is a continuation of application No. 16/392,108, filed on Apr. 23, 2019, now abandoned.
Application No. 16/392,108 is a continuation of application No. 16/122,333, filed on Sep. 5, 2018, now abandoned.
Application No. 16/122,333 is a continuation of application No. 15/857,122, filed on Dec. 28, 2017, now abandoned.
Application No. 15/857,122 is a continuation of application No. 13/957,100, filed on Aug. 1, 2013, now Pat. No. 9,873,739.
(60) Provisional application No. 61/678,564, filed on Aug. 1, 2012.

(51) Int. Cl. C07K 16/26; A61K 38/10; A61K 39/00; A61K 39/395; A61K 45/06; C07K 7/08; C07K 16/18

(52) U.S. Cl. CPC C07K 16/26; A61K 38/10; A61K 39/39533; A61K 39/3955; A61K 45/06; C07K 7/08; C07K 16/18; A61K 2039/505; C07K 2317/34; C07K 2317/76

(57) ABSTRACT

The disclosure relates to compositions and methods of mitigating tissue damage and fibrosis in a patient by modulating latent transforming growth factor beta binding protein (LTBP4)-induced proteolysis of a TGFβ superfamily protein.

### CROSS REFERENCE TO RELATED APPLICATIONS

This application is a Continuation of U.S. application Ser. No. 19/281,249, filed Jul. 25, 2025, which is a Continuation of U.S. application Ser. No. 18/980,341, filed Dec. 13, 2024, which is a Continuation of U.S. application Ser. No. 18/646,068, filed Apr. 25, 2024, which is a Continuation of U.S. application Ser. No. 17/811,233, filed Jul. 7, 2022, which is a Continuation of U.S. application Ser. No. 17/536,768, filed Nov. 29, 2021, which is a Continuation of U.S. application Ser. No. 17/219,845, filed Mar. 31, 2021, which is a Continuation of U.S. application Ser. No. 16/994,225, filed Aug. 14, 2020, which is a Continuation of U.S. application Ser. No. 16/716,382, filed Dec. 16, 2019, which is a Continuation of U.S. application Ser. No. 16/392,108, filed Apr. 23, 2019, which is a Continuation of U.S. application Ser. No. 16/122,333, filed Sep. 5, 2018, which is a Continuation of U.S. application Ser. No. 15/857,122, filed Dec. 28, 2017, which is a Continuation of U.S. application Ser. No. 13/957,100, filed Aug. 1, 2013, which claims the priority benefit under 35 U.S.C. § 119 (e) of Provisional U.S. Patent Application No. 61/678,564, filed Aug. 1, 2012, the disclosure of which is incorporated herein by reference in its entirety.

### STATEMENT OF GOVERNMENT INTEREST

This invention was made with government support under Grant Number HL61322, awarded by the National Institutes of Health (NIH). The government has certain rights in the invention.

### FIELD

The disclosure relates to compositions and methods of mitigating tissue damage and fibrosis in a patient via latent transforming growth factor beta binding protein (LTBP4).

### SEQUENCE LISTING

This application contains, as a separate part of the disclosure, a Sequence Listing in computer-readable form (filename: 46577J_SeqListing.xml; created: Apr. 23, 2024; 170,014 bytes in size) which is incorporated by reference in its entirety.

### BACKGROUND

The transforming growth factor (TGF) beta superfamily proteins are key regulators of fibrosis in all parenchymal organs [Kisseleva et al., Proc Am Thorac Soc. 5:338-42 (2008)]. Duchenne Muscular Dystrophy (DMD) is characterized by progressive fibrosis that is accompanied by increased TGFβ signaling [Bernasconi et al., J Clin Invest. 96:1137-44 (1995); Chen et al., Neurology 65:826-34 (2005)]. In DMD, fibrosis not only contributes directly to muscle dysfunction but also inhibits regeneration. DMD is characterized by muscle membrane fragility that leads to progressive myofiber loss. With disease progression, DMD muscle is replaced by fibrosis. Although muscle is highly regenerative, regeneration in DMD is not sufficient to offset degeneration leading to muscle weakness. Glucocorticoid steroids are used to slow progression in DMD, but use of steroids is complicated by side effects including osteoporosis and weight gain (Bushby et al., 2010). Experimental therapies for DMD include approaches to increase dystrophin expression, modulate the inflammatory response, promote muscle growth and reduce fibrosis [Bushby et al., Lancet 374:1849-56 (2009)].

In recent years, biological compounds such as antibodies have shown efficacy for treating chronic diseases. For example, antibodies directed against TNFα (infliximab) or anti-TNF receptor (etanercept) are now in wide use for rheumatoid arthritis and other related disorders. While initially developed for its anti-cancer activity, the anti-VEGF antibody is now used to treat macular degeneration (bevacizumab). Thus, long-term use with biological compounds can be effective and safe. Consistent with therapeutic approaches comprising the administration of a biological compound such as an antibody is the fact that antibodies are readily detected in the matrix of dystrophic muscle, such as the muscle of DMD patients.

A number of approaches, including but not limited to angiotensin inhibition, either through the converting enzyme or the angiotensin receptor, aldosterone inhibition, and inhibition by antibodies directed against TGFβ have been or are being tested to reduce fibrosis in DMD. [Cohn et al., Nat Med. 13:204-10 (2007); Rafael-Fortney et al., Circulation. 124:582-8 (2011); Nelson et al., Am J Pathol. 178:2611-21 (2011)]. A major limitation of these approaches is that these drugs are systemically active and often have unwanted effects such as reduced blood pressure. Given the relative hypotension of DMD patients, especially advanced DMD patients, such approaches are limited.

### SUMMARY

Disclosed herein are compositions and methods for treating a transforming growth factor beta superfamily protein-related disease. Compositions according to the disclosure modulate the activity and/or proteolysis of latent TGFβ binding protein 4 (LTBP4). Methods according to the disclosure comprise administration of an effective amount of a modulator of LTBP4, with that effective amount being an amount sufficient to prevent, delay onset and/or treat a disorder according to the disclosure. The compositions and methods provided by the disclosure will improve one or more symptoms associated with disorders according to the disclosure in afflicted individuals, thereby improving their quality of life while alleviating the financial, psychological and physical burdens imposed on modern healthcare systems.

Accordingly, in one aspect the disclosure provides a method of treating a patient having a transforming growth factor beta (TGFβ) superfamily protein-related disease, comprising administering an effective amount of an agent that modulates proteolysis of latent TGFβ binding protein 4 (LTBP4) to a patient in need thereof.

A related aspect of the disclosure provides methods of delaying onset or preventing a transforming growth factor beta (TGFβ) superfamily protein-related disease, comprising administering an effective amount of an agent that modulates proteolysis of latent TGFβ binding protein 4 (LTBP4) to a patient in need thereof.

In various embodiments of the foregoing methods, the patient has a disease selected from the group consisting of Duchenne Muscular Dystrophy, Limb Girdle Muscular Dystrophy, Becker Muscular Dystrophy, myopathy, cystic fibrosis, pulmonary fibrosis, cardiomyopathy, acute lung injury, acute muscle injury, acute myocardial injury, radiation-induced injury and colon cancer.

In further embodiments, the agent is selected from the group consisting of an antibody, an inhibitory nucleic acid and a peptide.

In further aspects of the disclosure, the methods disclosed herein further comprise administering an effective amount of a second agent, wherein the second agent is selected from the group consisting of a modulator of an inflammatory response, a promoter of muscle growth, a chemotherapeutic agent and a modulator of fibrosis.

Another aspect of the disclosure is drawn to a method of treating a patient having a transforming growth factor beta (TGFβ)-related disease, comprising administering to the patient an effective amount of an agent that upregulates the activity of latent TGFβ binding protein 4 (LTBP4).

A further aspect of the disclosure provides a method of delaying onset or preventing a transforming growth factor beta (TGFβ)-related disease, comprising administering to the patient an effective amount of an agent that upregulates the activity of latent TGFβ binding protein 4 (LTBP4).

In some embodiments of the methods, LTBP4 interacts with a TGFβ superfamily protein, and in still further embodiments the TGFβ superfamily protein is selected from the group consisting of TGFβ, a growth and differentiation factor (GDF), activin, inhibin, and a bone morphogenetic protein. In specific embodiments, the GDF is myostatin.

In additional embodiments, the agent is selected from the group consisting of a peptide, an antibody and a polynucleotide capable of expressing a protein having LTBP4 activity, each as disclosed herein. In some embodiments, the polynucleotide is contained in a vector and in further embodiments the vector is a viral vector. The disclosure further contemplates embodiments wherein the viral vector is selected from the group consisting of a herpes virus vector, an adeno-associated virus (AAV) vector, an adeno virus vector, and a lentiviral vector. In one embodiment, the AAV vector is recombinant AAV9.

In some embodiments, the compositions and methods disclosed herein are for treating a transforming growth factor beta-related disease in a patient. In particular embodiments, the patient suffers from a disease selected from the group consisting of Duchenne Muscular Dystrophy (DMD), Limb Girdle Muscular Dystrophy (LGMD), Becker Muscular Dystrophy (BMD), myopathy, cystic fibrosis, pulmonary fibrosis, cardiomyopathy, acute lung injury, acute muscle injury, acute myocardial injury, radiation-induced injury and colon cancer.

An additional aspect of the disclosure is drawn to methods as disclosed above that further comprise administering a therapeutically effective amount of a second agent that is selected from the group consisting of a modulator of an inflammatory response, a promoter of muscle growth, a chemotherapeutic agent and a modulator of fibrosis.

In some embodiments, an isolated antibody is provided that specifically binds to a peptide comprising any one of the sequences set forth in SEQ ID NOs: 2-5. In further embodiments, the disclosure provides an isolated antibody that specifically binds to a peptide that is at least 70% identical to a peptide comprising any one of the sequences set forth in SEQ ID NOs: 2-5, wherein the antibody retains an ability to specifically bind to LTBP4 and to decrease the susceptibility of LTBP4 to proteolysis.

Still further embodiments of the disclosure provide a peptide comprising the sequence as set out in SEQ ID NOs: 2-5, or a peptide that is at least 70% identical to any one of the sequences as set out in SEQ ID NO: 2-5 that retains an ability to act as a substrate for a protease.

In another aspect, a pharmaceutical formulation is provided comprising an effective amount, such as a therapeutically effective amount, of an antibody and/or peptide of the disclosure, and a pharmaceutically acceptable carrier or diluent.

A further aspect of the disclosure provides a kit comprising an effective amount, such as a therapeutically effective amount, of an antibody and/or peptide of the disclosure, a pharmaceutically acceptable carrier or diluent and instructions for use.

In some embodiments, the formulation or the kit of the disclosure further comprises an effective amount, such as a therapeutically effective amount, of a second agent, wherein the second agent is selected from the group consisting of a modulator of an inflammatory response, a promoter of muscle growth, a chemotherapeutic agent and a modulator of fibrosis.

Other features and advantages of the present disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 depicts a model for action of LTBP4 (latent TGFβ binding protein 4). LTBP4 binds directly to TGFβ family member proteins. In the extracellular matrix, the complex of LTBP4 protein and TGFβ forms the large latent complex. With proteolysis, LTBP4 undergoes a conformational change which releases TGFβ, thereby making it available for release and binding TGFβ receptors on neighboring cells. TGFβ binding to its receptor results in TGFβ signaling in cells.

FIG. 2 depicts the gene structure of LTBP4. An insertion deletion polymorphism in Ltbp4 alters the proline-rich region in mice. The N-terminus of LTBP binds the extracellular matrix (ECM). The LTBP4 protein is composed of multiple epidermal growth factor (EGF) repeats interspersed with motifs containing 8 cysteine residues (8-Cys). The third 8-cys repeat binds TGFβ directly. The proline-rich region (labeled horizontal rectangle) separates the matrix-binding domain from the remainder of the protein. Mouse129 is protected against muscular dystrophy because of insertion of 12 amino acids in the proline-rich region. Muscular dystrophy in D2 strains of mice is more severe. Rat, dog, cow, and humans each harbor a larger deletion of the proline-rich region of LTBP4.

FIG. 3 depicts results of studies using fragments of human and mouse LTBP4 that were expressed and digested. Human LTBP4 is more readily cleaved than murine LTBP4. The amino acid positions indicated for TP and TP2E are based on the human isoform a LTBP4 sequence (SEQ ID NO: 1).

FIG. 4 depicts results of studies using a blocking antibody that was designed to recognize and bind the proline-rich region (Y) of LTBP4. When incubated with cell lysates expressing LTBP4, the presence of the antibody inhibits cleavage by plasmin. A nonspecific antibody did not inhibit cleavage.

FIG. 5 depicts that the proline-rich region of human LTBP4 is more easily cleaved than murine LTBP4.

FIG. 6 shows results of a study using a blocking antibody that inhibited cleavage of full-length LTBP4. A nonspecific blocking antibody showed no effect. Assays were conducted in triplicate with significant inhibition of proteolysis observed.

FIG. 7 shows results of a study wherein a bacterial artificial transgene expressing human LTBP4 (hLTBP4 Tg) was crossed into the mouse mdx model of Duchenne Muscular Dystrophy. (A) hLTBP4/mdx mice have enhanced fibrosis in their muscles as determined grossly through histology and by direct quantitation. When quantified, fibrotic area was increased in hLTBP4/mdx mice compared to littermate mdx mice. (B) hLTBP4/mdx mice have reduced grip strength. Grip strength was compared between hLTBP4/mdx mice and mdx mice to determine whether the human LTBP4 gene worsens the muscle disease seen in mdx mice. hLTBP4/mdx mice are weaker than mdx littermates (*). Grip strength was measured using the Treat NMD standard protocols.

FIG. 8 shows that LTBP4 forms a complex with myostatin. HEK293 cells were transfected with LTBP4 and epitope-tagged myostatin. LTBP4 was precipitated with two different anti-LTBP4 antibodies (lanes 3 and 5), and the precipitate was then immunoblotted with anti-myc antibody. Unprocessed myostatin was detected in the immunoprecipitate (arrow). The upper band in lanes 1 and 2 that migrates above 50 KDa is endogenous c-myc, which is 63 KDa.

FIG. 9 depicts the results of experiments testing the effects of cardiotoxin on both wild-type mice and transgenic mice that express human LTBP4. A) transgenic mice displayed enhanced injury after cardiotoxin injury seen as greater inflammatory mononuclear cell infiltrate and fibrosis and fat deposition into the injured muscle. B) LTBP4 protein levels are increased after injury.

FIG. 10 shows that anti-LTBP4 antibodies mitigate muscle injury in vivo. Compared to PBS-injected mice, LTBP4-831 antibody-treated mice showed reduced central nucleation (panel A) and reduced fibrosis (panel B) following cardiotoxin injection.

FIG. 11 shows that increased TGFβ signaling is associated with increased macrophage infiltration in hLTBP4/mdx muscle compared to mdx muscle. A) Muscles were stained with antibodies to activated macrophages using the F4/80 antibody. B) hLTBP4/mdx muscle shows an increase in cleaved LTBP4 protein compared to mdx, while little LTBP4 protein is seen in wild type and hLTBP4 muscle in the absence of injury or muscular dystrophy. C) Proteolytic cleavage and a conformational change in LTBP4 is associated with TGFβ release.

### DETAILED DESCRIPTION

The transforming growth factor beta (TGFβ) superfamily consists of more than 40 members including TGFβ, activins, inhibins, growth differentiation factors and bone morphogenetic proteins (BMPs). All members of this family share common sequence elements and structural motifs. They are multifunctional regulators of cell division, differentiation, migration, adhesion, organization and death, promoting extracellular matrix (ECM) production, tissue homeostasis and embryogenesis [Massague et al., Genes Dev 19:2783-810 (2005); Javelaud et al., Int J Biochem Cell Biol 36:1161-5 (2004); Moustakas et al., Immunol Lett 82:85-91 (2002)]. Among these proteins, TGFβ has a crucial role in tissue homeostasis and the disruption of the TGFβ pathway has been implicated in many human diseases, including cancer, autoimmune, fibrotic, and cardiovascular diseases [Ruiz-Ortega et al., Cardiovascular Research 74:196-206 (2007)].

TGFβ is synthesized as an inactive protein, named latent TGFβ, which consists of a main region and a latency associated peptide (LAP). This protein interacts with the latent TGFβ binding proteins (e.g., LTBP4) and is anchored in the extracellular matrix (ECM). TGFβ is activated following proteolysis of LTBP4, which results in release of TGFβ. Specifically, and as disclosed herein, the proline-rich region of LTBP4 is susceptible to proteolysis by a protease, and this proteolysis leads to release and activation of TGFβ.

Active TGFβ then binds its receptors and functions in autocrine and paracrine manners to exert its biological and pathological activities via Smad-dependent and independent signaling pathways [Lan, Int J Biol Sci 7 (7): 1056-1067 (2011); Derynck et al., Nature. 425:577-84 (2003)].

Thus, inhibition of the proteolysis of LTBP4 will inhibit the release of bound TGFβ, and the resulting sequestration of TGFβ will inhibit the downstream signaling effects of TGFβ, resulting in mitigation of TGFβ-related disease.

The working examples and experimental data disclosed therein demonstrate that the proline-rich region of LTBP4 is susceptible to proteolysis. These results support therapeutics and therapies directed to modulating the proteolysis of LTBP4 in a patient having a TGFβ-related disease.

The experimental results disclosed herein also demonstrate that proteolysis of LTBP4 can be inhibited by antibodies. Inhibition of LTBP4 proteolysis using pharmacological approaches is expected to provide an effective approach to the treatment of TGFβ-related diseases.

Experimental results disclosed herein additionally demonstrate that a fragment of human LTBP4 is more susceptible to proteolysis than the mouse LTBP4 sequence. Consequently, a phenomenon elucidated in the mouse is mirrored in humans, and inhibition of LTPB4 proteolysis is expected to provide an effective treatment for TGFβ-related diseases.

Unless otherwise defined herein, scientific and technical terms employed in the disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

As used in the disclosure, the term “treating” or “treatment” refers to an intervention performed with the intention of preventing the further development of or altering the pathology of a disease or infection. Accordingly, “treatment” refers to both therapeutic treatment and prophylactic or preventative measures. Of course, when “treatment” is used in conjunction with a form of the separate term “prophylaxis,” it is understood that “treatment” refers to the narrower meaning of altering the pathology of a disease or condition. “Preventing” refers to a preventative measure taken with a subject not having a condition or disease. A therapeutic agent may directly decrease the pathology of a disease, or render the disease more susceptible to treatment by another therapeutic agent(s) or, for example, the host's immune system. Treatment of patients suffering from clinical, biochemical, or subjective symptoms of a disease may include alleviating one or more of such symptoms or reducing the predisposition to the disease. Improvement after treatment may be manifested as a decrease or elimination of one or more of such symptoms.

As used herein, the phrase “effective amount” is meant to refer to an amount of a therapeutic (i.e., a therapeutically effective amount), prophylactic (i.e., a prophylactically effective amount), or symptom-mitigating (i.e., a symptom-mitigating effective amount) compound (e.g., agent or second agent) sufficient to modulate proteolysis of latent TGFβ binding protein 4 (LTBP4), such as would be appropriate for an embodiment of the disclosure in eliciting the desired therapeutic, prophylactic, or symptom-mitigating effect or response, including alleviating one or more of such symptoms of disease or reducing the predisposition to the disease.

As used herein, “hybridization” means the pairing of substantially complementary strands of polymeric compounds. One mechanism of pairing involves hydrogen bonding, which may be Watson-Crick, Hoogsteen or reversed Hoogsteen hydrogen bonding, between complementary nucleotide bases (nucleotides) of the strands of polymeric compounds. For example, adenine and thymine are complementary nucleotides which pair through the formation of hydrogen bonds. Hybridization can occur under varying circumstances.

An antisense compound is “specifically hybridizable” when binding of the compound to the target nucleic acid interferes with the normal function of the target nucleic acid to cause a modulation of function and/or activity, and there is a sufficient degree of complementarity to avoid non-specific binding of the antisense compound to non-target nucleic acid sequences under conditions in which specific binding is desired, i.e., under physiological conditions in the case of in vivo applications such as therapeutic treatment, and under conditions in which assays are performed in the case of in vitro assays.

As used herein, the phrase “stringent hybridization conditions” or “stringent conditions” refers to conditions under which a compound (e.g., agent) disclosed herein will hybridize to its target sequence, but to a minimal number of other sequences. Stringent conditions are sequence-dependent and will be different in different circumstances and in the context of this disclosure, “stringent conditions” under which polymeric compounds hybridize to a target sequence are determined by the nature and composition of the polymeric compounds and by the application(s) involved. In general, stringent hybridization conditions comprise low concentrations (<0.15M) of salts with inorganic cations such as Na⁺⁺ or K⁺⁺ (i.e., low ionic strength), temperatures higher than 20° C.-25° C. below the Tₘ of the polymeric compound: target sequence complex, and the presence of denaturants such as formamide, dimethylformamide, dimethyl sulfoxide, or the detergent sodium dodecyl sulfate (SDS). An example of a set of high stringency hybridization conditions is 0.1× sodium chloride-sodium citrate buffer (SSC)/0.1% (w/v) SDS at 60° C. for 30 minutes.

“Complementary,” as used herein, refers to the capacity for precise pairing between two nucleotides on one or two polymeric strands. Consistent with Watson-Crick base pairing rules (A binds T or U; G binds C; where A, G, C, T and U are the conventional ribo-, or deoxyribo-, nucleotide monophosphates). “Specifically hybridizable” and “complementary” are terms which are used to indicate a sufficient degree of precise nucleotide pairing or complementarity over a sufficient number of nucleotides such that stable and specific binding occurs between the polymeric compound and a target nucleic acid. The terms thus allow for base pairing gaps, but not to the extent that it prevents stable and specific binding.

It is understood in the art that the sequence of a polymeric compound need not be 100% complementary to that of its target nucleic acid to be specifically hybridizable. Moreover, a polynucleotide may hybridize over one or more segments such that intervening or adjacent segments are not involved in the hybridization event (e.g., a loop structure, mismatch or hairpin structure). The polymeric compounds of the present disclosure comprise at least about 70%, or at least about 75%, or at least about 80%, or at least about 85%, or at least about 90%, or at least about 95%, or at least about 99% sequence complementarity to a target region, within the target nucleic acid sequence to which they are targeted. For example, an antisense compound in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, an antisense compound which is 18 nucleotides in length having 4 (four) noncomplementary nucleotides which are flanked by two regions of complete complementarity with the target nucleic acid would have 77.8% overall complementarity with the target nucleic acid and would thus fall within the scope of the present disclosure. Percent complementarity of an antisense compound with a region of a target nucleic acid can be determined by use of routine sequence comparison software and algorithms, e.g., BLAST programs (basic local alignment search tools) and PowerBLAST programs known in the art [Altschul et al., J. Mol. Biol., 215:403-410 (1990); Zhang and Madden, Genome Res., 7:649-656 (1997)]. Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman [Adv. Appl. Math., 2:482-489 (1981)].

As used herein, the term “(Tₘ)” means melting temperature and refers to the temperature, under defined ionic strength, pH, and nucleic add concentration, at which 50% of the polynucleotides complementary to the target sequence hybridize to the target sequence at equilibrium. Typically, stringent conditions will be those in which the salt concentration is at least about 0.01 to 1.0 M sodium ion concentration (or other salts) at pH 7.0 to 8.3 and the temperature is at least about 30° C. for short polynucleotides (e.g., 10 to 50 nucleotides). Stringent conditions may also be achieved with the addition of destabilizing agents such as formamide.

As used herein, “modulation” of an activity means either an increase (stimulation) or a decrease (inhibition) in that activity. For example, and without limitation, a modulation of proteolysis can mean either an increase in proteolysis or a decrease in proteolysis.

### Latent TGFβ Binding Protein 4 (Ltbp4)

The present disclosure is directed in part to Ltbp4, the gene encoding latent TGFβ binding protein (LTBP4; GenBank Accession Number NP_001036009.1; SEQ ID NO: 1), which was identified in a genetic screen as a major genetic modifier of muscular dystrophy [Heydemann et al., J Clin Invest. 119:3703-12 (2009)]. This genetic screen was conducted using mice lacking the dystrophin-associated protein, γ-sarcoglycan (Sgcg null mice). The Sgcg model of limb girdle muscular dystrophy (LGMD) was selected because there was ample evidence from human LGMD of the importance of genetic modifiers affecting the severity of this disease [McNally et al., Am J Hum Genet. 59:1040-7 (1996)]. It was surprisingly found that modifiers identified for sarcoglycan-mediated muscular dystrophy similarly modify DMD. Disruption of the dystrophin glycoprotein complex, either in DMD or the sarcoglycan-associated LGMDs, leads to a fragile muscle membrane, enhanced myofiber breakdown, and replacement of normal muscle tissue by fibrosis. Early in pathology, fibrotic replacement is minimal, but in the advanced DMD patient, the muscle is nearly completely replaced by fibrosis.

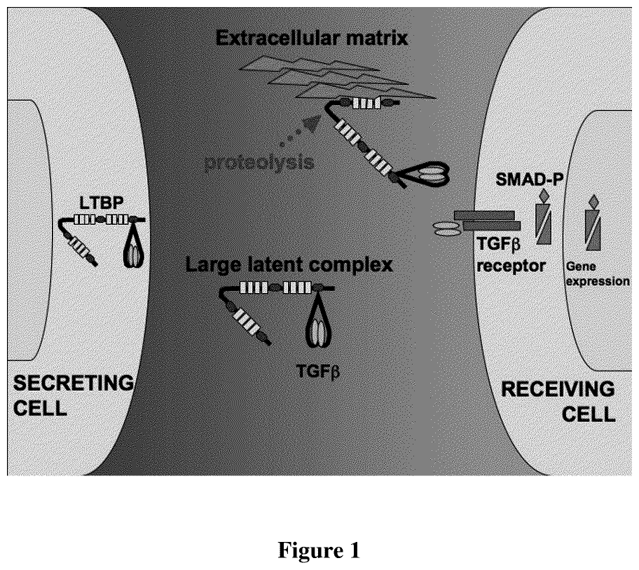

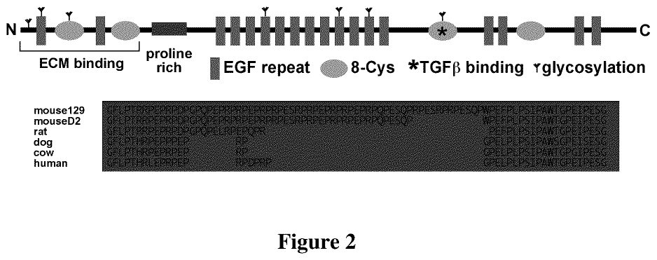

LTBP4 is located on human chromosome 19q13.1-q13.2, and is an extracellular matrix protein that binds and sequesters TGFβ (FIG. 1). LTBP4 modifies murine muscular dystrophy through a polymorphism in the Ltbp4 gene. There are two common variants of the Ltbp4 gene in mice. Most strains of mice, including the mdx mouse, have the Ltbp4 insertion allele (Ltbp4^{I/I}). Insertion of 36 base pairs (12 amino acids) into the proline-rich region of LTBP4 encoded by Ltbp4^{I/I} leads to milder disease. Deletion of 36 bp/12aa in the proline-rich region is associated with more severe disease (Ltbp4^{D/D}) (FIG. 2). It was found that the Ltbp4 genotype correlated strongly with two different aspects of muscular dystrophy pathology, i.e., membrane leakage and fibrosis, and these features define DMD pathology.

To assess muscle membrane leakage, Evans blue dye (EBD), which can complex with serum albumin, and thus is a measure of membrane permeability, was used. EBD is injected intraperitoneally and muscles from the injected animals are harvested approximately 8-40 hours later. Muscle membrane leakage was assessed by determining the amount of EBD in multiple different muscle groups, including quadriceps and other skeletal muscles. Hydroxyproline content was measured to quantify fibrosis, and this assay was also performed on multiple different muscle groups. The Ltbp4 genotype was found to account for nearly 40% of the variance in membrane leakage in quadriceps muscle [Swaggart et al., Physiol Genomics 43:24-31 (2011)]. Similarly, the Ltbp4 genotype also highly correlated with fibrosis in limb-based skeletal muscles where it also accounted for a significant amount of the variance. Ltbp4 is an unusually strong genetic modifier and acts both on membrane fragility as well as fibrosis. Accordingly, the present disclosure identifies LTBP4 as a target for therapy because it will stabilize the plasma membrane in addition to reducing fibrosis in patients in need thereof.

As discussed hereinabove, LTBP4 is a matrix-associated protein that binds and sequesters TGFβ. TGFβ in this form is the large latent complex, which requires further proteolysis to become fully active. It is expected that matrix-bound latent TGFβ is the least active form with regard to receptor engagement, and therefore represents an ideal step at which to inhibit TGFβ release. LTBP4, the fourth member of the LTBP carrier protein family, is highly expressed in heart, muscle, lung and colon [Saharinen et al., J Biol Chem. 273:18459-69 (1998)]. LTBP4 protein, like other members of this family, can be proteolyzed with plasmin, which results in TGFβ release [Saharinen et al., J Biol Chem. 273: 18459-69 (1998); Ge et al., J Cell Biol. 175:111-20 (2006)]. The 12-amino-acid insertion/deletion alters the susceptibility of LTBP4 to proteolysis, which in turn alters TGFβ release and its ability to bind TGFβ receptors and activate signaling. It is disclosed herein that inhibiting LTBP4 cleavage will hold TGFβ inactive and limit the downstream effects of TGFβ release.

### Agents

Methods of the disclosure contemplate treating a patient having a TGFβ-related disease comprising administering to the patient an effective amount of an agent that modulates proteolysis of LTBP4.

The term “agent” in this context refers to an antibody, an inhibitory nucleic acid, a peptide, and combinations thereof.

### Antibodies

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)₂, Fv, single chain antibodies, diabodies), camel bodies and recombinant peptides comprising the foregoing provided they exhibit the desired biological activity. Antibody fragments may be produced using recombinant DNA techniques or by enzymatic or chemical cleavage of intact antibodies and are described further below. Non-limiting examples of monoclonal antibodies include murine, chimeric, humanized, human, and human-engineered immunoglobulins, antibodies, chimeric fusion proteins having sequences derived from immunoglobulins, or muteins or derivatives thereof, each described further below. Multimers or aggregates of intact molecules and/or fragments, including chemically derivatized antibodies, are contemplated. Antibodies of any isotype class or subclass are contemplated.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. In contrast to conventional (polyclonal) antibody preparations that typically include different antibodies directed against different determinants (epitopes), each monoclonal antibody is directed against a single determinant on the antigen. In addition to their specificity, monoclonal antibodies are advantageous in that they are synthesized in a homogeneous culture, uncontaminated by other immunoglobulins with different specificities and characteristics.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the disclosure may be made by the hybridoma method first described by Kohler et al., Nature 256:495 (1975), or may be made by recombinant DNA methods (see, e.g., U.S. Pat. No. 4,816,567, incorporated herein by reference). The “monoclonal antibodies” may also be recombinant, chimeric, humanized, human, Human Engineered™, or antibody fragments, for example.

Antibodies described herein are discussed in Example 3. In certain embodiments, a variant of an antibody of the disclosure is contemplated. By “variant” is meant an antibody comprising one or more amino acid substitutions, amino acid deletions, or amino acid additions to a reference amino acid sequence. Variants include, but are not limited to, antibodies having an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any of the amino acid sequences of an antibody provided herein, provided that the antibody variant retains the ability to block and/or inhibit the proteolysis of LTBP4.

In further embodiments, an anti-LTBP4 antibody described herein specifically binds at least one peptide selected from the group consisting of peptides having a sequence set forth in SEQ ID NOs: 2-5, or a peptide selected from the group consisting of peptides having a sequence at least 70% identical to a peptide having a sequence set forth in SEQ ID NOs: 2-5. In additional embodiments, an anti-LTBP4 antibody described herein binds at least one epitope of LTBP4 with an affinity of 10⁻⁶ M, 10⁻⁷ M, 10⁻⁸ M, 10⁻⁹ M, 10⁻¹⁰ M, 10⁻¹¹ M, or 10⁻¹² M or less (lower meaning higher binding affinity), or optionally binds all of LTBP4 with an affinity of 10⁻⁶ M, 10⁻⁷ M, 10⁻⁸ M, 10⁻⁹ M 10⁻¹⁰ M, 10⁻¹¹ M, or 10⁻¹² M or less. In other embodiments, an antibody described herein “specifically binds” to LTBP4 with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% higher affinity compared to binding to a non-target protein.

Antibodies described hereinbelow are suitable for use in the methods described herein. Additional antibodies are also contemplated, provided the antibody possesses the property of modulating the proteolysis or upregulating the activity of LTBP4. Such antibodies may, for example, be humanized according to known techniques and modified and/or formulated to allow delivery and intracellular contact with LTBP4.

### Peptides

The disclosure provides peptides that have the ability to act as a substrate for a protease (i.e., “a protease-substrate peptide”). The protease, as discussed herein, means a protease that can cleave LTBP4. In one embodiment, the protease is a serine protease. In further embodiments, the protease is selected from the group consisting of plasmin, leukocyte elastase, pancreatic elastase, human mast cell chymase, trypsin, chymotrypsin, pepsin and papain.

The ability of a peptide to act as a substrate for a protease can be readily determined by one of ordinary skill in the art. By way of non-limiting example, a peptide can be tested in vitro by incubating a labeled LTBP4 protein (or a labeled fragment thereof) with a candidate peptide and a serine protease. The label can be any detectable label known in the art, and in one embodiment is a radioactive label. Following incubation and subsequent gel electrophoresis, it can be determined whether the LTBP4 protein (or fragment thereof) was refractory to proteolysis based on the size of the protein on the gel. If the LTBP4 protein was not protected from proteolysis by the peptide, the radioactive band on the gel will be smaller than one would expect for a full-length LTBP4 protein. Thus, while peptide sequences disclosed herein are contemplated for use according to the methods of the disclosure, additional peptides are also contemplated, with the proviso being their ability to act as a substrate for a protease in a manner that renders them an inhibitor of LTBP4 proteolysis.

Use of one or more peptides or antibodies of the disclosure, each of which has an ability to act either as a substrate for a protease (peptide) or to act as an inhibitor of proteolysis (antibody), is expected to upregulate the activity of LTBP4 compared to the activity of LTBP4 in the absence of the one or more peptides. In this context, upregulation of LTBP4 activity results from its protection from proteolysis via the action of the one or more peptides and/or antibodies of the disclosure. The downstream effect of this upregulation of LTBP4 activity is the concomitant downregulation of TGFβ signaling. Intact LTBP4 will continue to bind and sequester TGFβ and thus prevent its release and subsequent downstream effects. Thus, in various embodiments, the upregulation of LTBP4 activity is measured by quantitating TGFβ signaling. Methods of quantitating TGFβ signaling are known to those of skill in the art, and include determination of Smad signaling from a biological sample obtained from a patient. It is contemplated that, in some embodiments, Smad signaling in a patient being administered one or more agent(s) and/or additional agent(s) of the disclosure is reduced by at least about 1% and up to about 5%, about 10%, about 20%, about 30%, about 40% or about 50% relative to a patient not so treated. In further embodiments, Smad signaling in a patient being administered one or more agent(s) and/or additional agent(s) of the disclosure is reduced by at least about 10% and up to about 20%, about 50%, about 70%, about 80%, about 90%, about 99% or more relative to a patient not so treated. In specific embodiments, Smad signaling in a patient being administered one or more agent(s) and/or additional agent(s) of the disclosure is reduced by at least about 1%, about 2%, about 5%, about 10%, about 20%, about 25%, about 30%, about 35%, about 40%, about 50%, about 60%, about 70%, about 80%, about 90%, about 95%, about 99% or more relative to a patient not so treated.

Peptides described above (i.e., peptide inhibitors of LTBP4 proteolysis) are set forth in Example 3 and Table 1. Thus, in certain embodiments, the peptide comprises or consists of the amino acid sequence of any one of SEQ ID NOs: 2-5 or a variant of any of the foregoing. By “variant” is meant a peptide comprising one or more amino acid substitutions, amino acid deletions, or amino acid additions to a reference amino acid sequence (e.g., any one of SEQ ID NOs: 2-5). Variants include, but are not limited to, peptides having an amino acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any of the amino acid sequences provided herein while retaining the ability to act as a substrate for a protease.

In one aspect, the peptide consists of 35 amino acids or less. In various embodiments, the peptide comprises 15-35 amino acid residues (e.g., 15, 16, 17, 18, 19, 20, 21, 22, 23, 24, 25, 26, 27, 28, 29, 30, 31, 32, 33, 34, or 35 amino acid residues). It is also contemplated that a peptide described herein comprising one or more deletions is suitable in the context of the disclosure so long as the peptide can act as a substrate for a protease. In some embodiments, amino acids are removed from within the amino acid sequence, at the N-terminus, and/or at the C-terminus. Such peptide fragments can comprise 3-14 amino acid residues (e.g., 3, 4, 5, 6, 7, 8, 9, 10, 11, 12, 13, or 14 amino acid residues).

Optionally, the peptide comprises one or more amino acid substitutions (with reference to any of the amino acid sequences provided herein) that do not destroy the ability of the peptide to act as a substrate for a protease. Amino acid substitutions include, but are not limited to, those which: (1) reduce susceptibility to proteolysis, (2) reduce susceptibility to oxidation, (3) alter binding affinities, and/or (4) confer or modify other physiochemical or functional properties on a peptide. In one aspect, the substitution is a conservative substitution, wherein an amino acid residue is replaced with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and side chains with aromatic character (e.g., tyrosine, phenylalanine, tryptophan, and histidine). It will be appreciated, however, that a practitioner is not limited to conservative substitutions, so long as the resulting peptide retains the ability to act as a substrate, in whole or in part, for a protease. The disclosure also embraces protease-substrate peptides comprising atypical, non-naturally occurring amino acids, which are well known in the art. The individual amino acids may have either L or D stereochemistry when appropriate, although the L stereochemistry is typically employed for all of the amino acids in the peptide.

The disclosure further includes protease-substrate peptide variants comprising one or more amino acids inserted within an amino acid sequence provided herein and/or attached to the N-terminus or C-terminus. In some embodiments, the peptide further comprises one or more amino acids that facilitate synthesis, handling, or use of the peptide including, but not limited to, one or two lysines at the N-terminus and/or C-terminus to increase solubility of the peptide. Suitable fusion proteins include, but are not limited to, proteins comprising a peptide linked to another polypeptide, a polypeptide fragment, or amino acids not generally recognized to be part of the protein sequence. In some embodiments, a fusion peptide comprises the entire amino acid sequences of two or more peptides or, alternatively, comprises portions (fragments) of two or more peptides. In addition to all or part of the peptides described herein, a fusion protein optionally includes all or part of any suitable peptide comprising a desired biological activity/function. Indeed, in some embodiments, a peptide is operably linked to, for instance, one or more of the following: a peptide with long circulating half-life, a marker protein, a peptide that facilitates purification of the protease-substrate peptide, a peptide sequence that promotes formation of multimeric proteins, or a fragment of any of the foregoing. In one embodiment, two or more protease-substrate peptides are fused together, linked by a multimerization domain, or attached via chemical linkage to generate a protease-substrate peptide complex. The protease-substrate peptides may be the same or different.

“Derivatives” are also contemplated by the disclosure and include protease-substrate peptides that have been chemically modified in some manner distinct from addition, deletion, or substitution of amino acids. In this regard, a peptide provided herein is chemically bonded with polymers, lipids, other organic moieties, and/or inorganic moieties. Derivatives are prepared in some situations to increase solubility, absorption, or circulating half-life. Various chemical modifications eliminate or attenuate any undesirable side effect of the agent. In this regard, the disclosure provides protease-substrate peptides covalently modified to include one or more water-soluble polymer attachments. Useful polymers known in the art include, but are not limited to, polyethylene glycol, polyoxyethylene glycol, polypropylene glycol, monomethoxy-polyethylene glycol, dextran, cellulose, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of any of the foregoing. For further discussion of water soluble polymer attachments, see U.S. Pat. Nos. 4,640,835; 4,496,689; 4,301,144; 4,670,417; 4,791,192; and 4,179,337, incorporated herein by reference. In other embodiments, a peptide derivative includes a targeting moiety specific for a particular cell type, tissue, and/or organ. Alternatively, the peptide is linked to one or more chemical moieties that facilitate purification, detection, multimerization, and/or characterization of peptide activity.

Derivatives also include peptides comprising modified or non-proteinogenic amino acids or a modified linker group [see, e.g., Grant, Synthetic Peptides: A User's Guide, Oxford University Press (1992)]. Modified amino acids include, for example, amino acids wherein the amino and/or carboxyl group is replaced by another group. Non-limiting examples include modified amino acids incorporating thioamides, ureas, thioureas, acylhydrazides, esters, olefines, sulfonamides, phosphoric acid amides, ketones, alcohols, boronic acid amides, benzodiazepines and other aromatic or non-aromatic heterocycles [see Estiarte et al., Burgers Medicinal Chemistry, 6th edition, Volume 1, Part 4, John Wiley & Sons, New York (2002)]. Modified amino acids are often connected to the peptide with at least one of the above-mentioned functional groups instead of an amide bond. Non-proteinogenic amino acids include, but are not limited, to β-alanine (β-Ala), norvaline (Nva), norleucine (Nle), 4-aminobutyric acid (γ-Abu), 2-aminoisobutyric acid (Aib), 6-aminohexanoic acid (8-Ahx), ornithine (orn), hydroxyproline (Hyp), sarcosine, citrulline, cysteic acid (Coh), cyclohexylalanine, methioninesulfoxide (Meo), methioninesulfone (Moo), homoserinemethylester (Hsm), propargylglycine (Eag), 5-fluorotryptophan (5Fw), 6-fluorotryptophan (6Fw), 3′,4′-dimethoxyphenyl-alanine (Ear), 3′,4′-difluorophenylalanine (Dff), 4′-fluorophenyl-alanine (Pff), 1-naphthyl-alanine (1Ni), 1-methyltryptophan (1Mw), penicillamine (Pen), homoserine (HSe), α-amino isobutyric acid, t-butylglycine, t-butylalanine, phenylglycine (Phg), benzothienylalanine (Bta), L-homo-cysteine (L-Hcys), N-methyl-phenylalanine (NMF), 2-thienylalanine (Thi), 3,3-diphenylalanine (Ebw), homophenylalanine (Hfe), s-benzyl-L-cysteine (Ece) and cyclohexylalanine (Cha). These and other non-proteinogenic amino acids may exist as D- or L-isomers and D-isomers of proteinogenic amino acids may also be found in derivatives.

Examples of modified linkers include, but are not limited to, the flexible linker 4,7,10-trioxa-1,13-tridecanediamine (Ttds), glycine, 6-aminohexanoic acid, beta-alanine, and combinations of Ttds, glycine, 6-aminohexanoic acid and beta-alanine.

Protease-substrate peptides are made in a variety of ways. In some embodiments, the peptides are synthesized by solid-phase synthesis techniques including those described in Merrifield, J. Am. Chem. Soc. 85:2149 (1963); Davis et al., Biochem. Intl. 10:394-414 (1985); Larsen et al., J. Am. Chem. Soc. 115:6247 (1993); Smith et al., J. Peptide Protein Res. 44:183 (1994); O'Donnell et al., J. Am. Chem. Soc. 118:6070 (1996); Stewart and Young, Solid Phase Peptide Synthesis, Freeman (1969); Finn et al., The Proteins, 3rd ed., vol. 2, pp. 105-253 (1976); and Erickson et al., The Proteins, 3rd ed., vol. 2, pp. 257-527 (1976). Alternatively, the protease-substrate peptide is expressed recombinantly by introducing a nucleic acid encoding a protease-substrate peptide into host cells that are cultured to express the peptide. Such peptides are purified from the cell culture using standard protein purification techniques.

The disclosure also encompasses a nucleic acid comprising a nucleic acid sequence encoding an antibody or protease-substrate peptide. Methods of preparing DNA and/or RNA molecules are well known in the art. In one aspect, a DNA/RNA molecule encoding an antibody or protease-substrate peptide provided herein is generated using chemical synthesis techniques and/or using polymerase chain reaction (PCR). If desired, an antibody and/or protease-substrate peptide coding sequence is incorporated into an expression vector. One of ordinary skill in the art will appreciate that any of a number of expression vectors known in the art are suitable in the context of the disclosure, such as, but not limited to, plasmids, plasmid-liposome complexes, and viral vectors. Any of these expression vectors are prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, the nucleic acid is operably linked to one or more regulatory sequences, such as a promoter, activator, enhancer, cap signal, polyadenylation signal, or other signal involved in the control of transcription or translation.

As with all binding agents and binding assays, one of skill in this art recognizes that the various moieties to which a binding agent should not detectably bind in order to be biologically (e.g., therapeutically) effective would be exhaustive and impractical to list. Therefore, when discussing a peptide, the term “specifically binds” refers to the ability of a peptide to bind (or otherwise inhibit) a protease involved in cleavage of LTBP4 with greater affinity than it binds to a non-target control protein that is not the protease. For example, the peptide may bind to the protease with an affinity that is at least, 5, 10, 15, 25, 50, 100, 250, 500, 1000, or 10,000 times greater than the affinity for a control protein. In some embodiments, the peptide binds the protease with greater affinity than it binds to an “anti-target,” a protein or other naturally occurring substance in humans wherein binding of the peptide might lead to adverse effects. Several classes of peptides are potential anti-targets. Because protease-substrate peptides are expected to exert their activity in the extracellular matrix, ECM proteins are contemplated as anti-targets.

Also specifically contemplated by the disclosure are peptides that elicit an immune response to LTBP4 in methods to modulate LTBP4 that involve the host immune system. Thus, in some aspects, a composition is provided that comprises a peptide of the disclosure for use as a vaccine in an individual. Vaccines often include an adjuvant. The compositions comprising one or more peptides described herein may also contain an adjuvant, or be administered with an adjuvant. Thus, the adjuvant may be administered with the peptide compositions or as part of the peptide compositions, before the peptide compositions, or after the peptide compositions.

A variety of adjuvants are suitable for use in combination with the peptide composition to elicit an immune response to the peptide. Preferred adjuvants augment the intrinsic response to an antigen without causing conformational changes in the antigen that affect the qualitative form of the response. Adjuvants for use in the methods disclosed herein include, but are not limited to, keyhole limpet hemocyanin (KLH), forms of alum (see below) and 3 De-O-acylated monophosphoryl lipid A (MPL or 3-DMP) [see GB 2220211]. Other suitable adjuvant include QS21, which is a triterpene glycoside or saponin isolated from the bark of the Quillaja Saponaria Molina tree found in South America [see Kensil et al., in Vaccine Design: The Subunit and Adjuvant Approach (eds. Powell and Newman, Plenum Press, NY, 1995); U.S. Pat. No. 5,057,540] and CpG [Bioworld Today, Nov. 15, 1998]. Still other suitable adjuvants are described in the following paragraph.

One class of suitable adjuvants, noted briefly above, is aluminum salts (alum), such as aluminum hydroxide, aluminum phosphate, and aluminum sulfate. Such adjuvants can be used with or without other specific immunostimulating agents such as MPL or 3-DMP, QS21, polymeric or monomeric amino acids such as polyglutamic acid or polylysine. Another class of suitable adjuvants is oil-in-water emulsion formulations [such as squalene or peanut oil], optionally in combination with immunological stimulants, such as monophosphoryl lipid A [see Stoute et al., N. Engl. J. Med. 336, 86-91 (1997)]. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetyl-muramyl-L-alanyl-D-isoglutaminyl-L-alanine-2-(1,2-dipalmitoyl-sn-glycero-3-(hydroxyphosphoryloxy)) ethylamide (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Additional oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as a Model 110Y microfluidizer (Microfluidics, Newton Mass.), (b) SAF, containing 10% Squalane, 0.4% Tween 80, 5% pluronic-blocked polymer L121, and thr-MDP, either microfluidized into a submicron emulsion or vortexed to generate a larger particle size emulsion, and (c) the Ribi adjuvant system (RAS), (Ribi Immunochem, Hamilton, Mont.) containing 2% squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™). Another class of suitable adjuvants is saponin adjuvants, such as Stimulon™ (QS21, Aquila, Worcester, Mass.) or particles generated therefrom such as ISCOMs (immunostimulating complexes) and ISCOMATRIX. Other adjuvants include Complete Freund's Adjuvant (CFA) and Incomplete Freund's Adjuvant (IFA). Any of the suitable adjuvants may include a cytokine, such as an interleukin (IL-1, IL-2, or IL-12), macrophage colony stimulating factor (M-CSF), tumor necrosis factor (TNF), or combinations of cytokines.

An adjuvant can be administered with a peptide composition of the disclosure as a single composition, or can be administered before, concurrent with or after administration of a peptide composition of the disclosure. Immunogen and adjuvant can be packaged and supplied in the same vial or can be packaged in separate vials and mixed before use. Immunogen and adjuvant are typically packaged with a label indicating the intended application, such as a therapeutic application. If immunogen and adjuvant are packaged separately, the packaging typically includes instructions for mixing before use. The choice of an adjuvant and/or carrier depends on the stability of the vaccine containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory agencies. For example, Complete Freund's adjuvant is not suitable for human administration, while alum, MPL and QS21 are suitable. Optionally, two or more different adjuvants can be used simultaneously, such as alum with MPL, alum with QS21, MPL with QS21, and alum, QS21 and MPL together. Also, Incomplete Freund's adjuvant can be used [Chang et al., Advanced Drug Delivery Reviews 32, 173-186 (1998)], optionally in combination with any of alum, QS21, and MPL and all combinations thereof.

### Inhibitory Nucleic Acids

By “inhibitory nucleic acid” is meant an RNA or DNA polynucleotide that binds to another RNA or DNA (target RNA, DNA). An inhibitory nucleic acid downregulates expression and/or function of a particular target polynucleotide. The definition is meant to include any foreign RNA or DNA molecule which is useful from a therapeutic, diagnostic, or other viewpoint. Such molecules include, for example, antisense polynucleotides such as RNA or DNA molecules, interference RNA (RNAi), micro RNA (miRNA), siRNA, enzymatic RNA, aptamers, ribozymes and other polymeric compounds that hybridize to at least a portion of the target polynucleotide or target polypeptide. As such, these compounds may be introduced in the form of single-stranded, double-stranded, triple-stranded, or partially single-stranded molecules, and the molecules may be linear or circular polymeric compounds.

The production and use of aptamers is known to those of ordinary skill in the art. In general, aptamers are nucleic acid- or peptide-binding species capable of tightly binding to and discreetly distinguishing target ligands [Yan et al., RNA Biol. 6 (3): 316-320 (2009), incorporated by reference herein in its entirety]. Aptamers, in some embodiments, may be obtained by a technique called the systematic evolution of ligands by exponential enrichment (SELEX) process [Tuerk et al., Science 249:505-10 (1990), U.S. Pat. Nos. 5,270,163, and 5,637,459, each of which is incorporated herein by reference in its entirety]. General discussions of nucleic acid aptamers are found in, for example and without limitation, Nucleic Acid and Peptide Aptamers: Methods and Protocols (Edited by Mayer, Humana Press, 2009) and Crawford et al., Briefings in Functional Genomics and Proteomics 2 (1): 72-79 (2003). In various aspects, an aptamer is between 10-100 nucleotides in length.

As used herein, the term “target polynucleotide” encompasses DNA, RNA (comprising pre-mRNA and mRNA) transcribed from such DNA, and also cDNA derived from such RNA, coding sequences, noncoding sequences, sense polynucleotides or antisense polynucleotides. The specific hybridization of a polymeric compound with its target nucleic acid interferes with the normal function of the target nucleic acid. This modulation of function of a target nucleic acid or polynucleotide by compounds that specifically hybridize to it is generally referred to as antisense modulation or inhibition. The functions of DNA to be interfered include, for example, replication and transcription. The functions of RNA to be interfered include all vital functions such as, for example and without limitation, translocation of the RNA to the site of protein translation, translation of protein from the RNA, splicing of the RNA to yield one or more mRNA species, catalytic activity which may be engaged in or facilitated by the RNA, and/or translation to express an encoded polypeptide. The overall effect of such modulation (e.g., inhibition) with target polynucleotide function is modulation of the expression of an encoded product or activity of the polynucleotide itself.

RNA interference “RNAi” is mediated by double-stranded RNA (dsRNA) molecules that have sequence-specific homology to their target nucleic acid sequence(s) [Caplen et al., Proc. Natl. Acad Sci. USA 98:9742-9747 (2001)]. In certain embodiments of the present disclosure, the mediators are “small interfering” RNA duplexes (siRNAs) of 5-25 nucleotides. The siRNAs are derived from the processing of dsRNA by an RNase enzyme known as Dicer [Bernstein et al., Nature 409:363-366 (2001)]. The siRNA duplex products are recruited into a multi-protein siRNA complex termed RISC (RNA-Induced Silencing Complex). Small interfering RNAs that can be used in accordance with the present disclosure can be synthesized and used according to procedures that are well known in the art and that will be familiar to the ordinarily skilled artisan. The siRNAs for use in the methods of the present disclosure suitably comprise between about 1 to about 50 nucleotides (nt). In non-limiting embodiments, siRNAs comprise about 5 to about 40 nt, about 5 to about 30 nt, about 10 to about 30 nt, about 15 to about 25 nt, or about 20-25 nucleotides.

Methods for inhibiting target polynucleotide expression are provided that include those wherein expression of the target polynucleotide is inhibited by at least about 5% and up to about 10%, 20%, 50% or 100%, at least about 5% and up to about 30%, 60%, 70% or 90%, or at least 10% and up to about 50%, 60%, 70%, 80%, 90% or 100%. In additional embodiments, expression of the target polynucleotide is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to target polynucleotide expression in the absence of an inhibitory nucleic acid. In other words, methods of inhibiting the expression or activity of a polynucleotide according to the disclosure result in essentially any degree of inhibition of expression of a target polynucleotide.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of specific inhibitory nucleic acid.

### Exon Skipping

Inhibitory nucleic acids are also contemplated for use in exon skipping. In general, exon skipping is a method in which inhibitory nucleic acids are designed to modulate the splicing of a gene of interest, resulting in mRNA transcripts that are able to make some level of gene product with some function. The inhibitory nucleic acids are, in various embodiments, short nucleic acid sequences designed to selectively bind to specific mRNA or pre-mRNA sequences to generate small double-stranded regions of the target mRNA. By binding to these regions and forming double strands at key sites where the spliceosome or proteins of the spliceosome would normally bind, mutated (frameshifting) exons are skipped and the remainder of the pre-mRNA is edited correctly in frame, albeit shorter.

Exon skipping is generally described in Hoffman et al. [The American J. of Path. 179 (1): 12-22 (2011)], Lu et al. [The American Soc. of Gene and Cell Therapy 19 (1): 9-15 (2011)], and U.S. Pat. Nos. 8,084,601, 7,960,541 and 7,973,015, all of which are incorporated herein by reference in their entireties.

Thus, the disclosure contemplates that skipping exons that encode the proline-rich region of LTBP4 will generate a protease-resistant protein. In some embodiments, one or more of exons 11, 12 and 13 of mouse LTBP4 (corresponding to exons 11, 12 and 13 in human LTBP4) are targeted for exon skipping. It is expected that skipping of the exons that encode part or all of the proline-rich region of LTBP4 will generate a protein that is resistant to protease activity.

### Compositions

Any of the agents and/or additional agents described herein (or nucleic acids encoding any of the agents and/or additional agents described herein) also is provided in a composition. In this regard, the agent and/or additional agent is formulated with a physiologically-acceptable (i.e., pharmacologically acceptable) carrier, buffer, or diluent, as described further herein. Optionally, the peptide is in the form of a physiologically acceptable salt, which is encompassed by the disclosure. “Physiologically acceptable salts” means any salts that are pharmaceutically acceptable. Some examples of appropriate salts include acetate, trifluoroacetate, hydrochloride, hydrobromide, sulfate, citrate, tartrate, glycolate, and oxalate.

### TGFβ-Related Diseases

TGFβ-related diseases contemplated for treatment according to the disclosure include Duchenne Muscular Dystrophy, Limb Girdle Muscular Dystrophy, Becker Muscular Dystrophy, myopathy, cystic fibrosis, pulmonary fibrosis, cardiomyopathy, acute lung injury, acute muscle injury, acute myocardial injury, radiation-induced injury, colon cancer, idiopathic pulmonary fibrosis, idiopathic interstitial pneumonia, autoimmune lung diseases, benign prostate hypertrophy, cerebral infarction, musculoskeletal fibrosis, post-surgical adhesions, liver cirrhosis, renal fibrotic disease, fibrotic vascular disease, neurofibromatosis, Alzheimer's disease, diabetic retinopathy, skin lesions, lymph node fibrosis associated with HIV, chronic obstructive pulmonary disease (COPD), inflammatory pulmonary fibrosis, rheumatoid arthritis; rheumatoid spondylitis; osteoarthritis; gout, other arthritic conditions; sepsis; septic shock; endotoxic shock; gram-negative sepsis; toxic shock syndrome; myofacial pain syndrome (MPS); Shigellosis; asthma; adult respiratory distress syndrome; inflammatory bowel disease; Crohn's disease; psoriasis; eczema; ulcerative colitis; glomerular nephritis; scleroderma; chronic thyroiditis; Grave's disease; Ormond's disease; autoimmune gastritis; myasthenia gravis; autoimmune hemolytic anemia; autoimmune neutropenia; thrombocytopenia; pancreatic fibrosis; chronic active hepatitis including hepatic fibrosis; renal fibrosis, irritable bowel syndrome; pyresis; restenosis; cerebral malaria; stroke and ischemic injury; neural trauma; Huntington's disease; Parkinson's disease; allergies, including allergic rhinitis and allergic conjunctivitis; cachexia; Reiter's syndrome; acute synoviitis; muscle degeneration, bursitis; tendonitis; tenosynovitis; osteopetrosis; thrombosis; silicosis; pulmonary sarcosis; bone resorption diseases, such as osteoporosis or multiple myeloma-related bone disorders; cancer, including but not limited to metastatic breast carcinoma, colorectal carcinoma, malignant melanoma, gastric cancer, and non-small cell lung cancer; graft-versus-host reaction; and auto-immune diseases, such as multiple sclerosis, lupus and fibromyalgia; viral diseases such as Herpes Zoster, Herpes Simplex I or II, influenza virus, Severe Acute Respiratory Syndrome (SARS) and cytomegalovirus.

As used herein, “cardiomyopathy” refers to any disease or dysfunction of the myocardium (heart muscle) in which the heart is abnormally enlarged, thickened and/or stiffened. As a result, the heart muscle's ability to pump blood is usually weakened, often leading to congestive heart failure. The disease or disorder can be, for example, inflammatory, metabolic, toxic, infiltrative, fibrotic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. The cardiac disorder may be pediatric in origin. Cardiomyopathy includes, but is not limited to, cardiomyopathy (dilated, hypertrophic, restrictive, arrhythmogenic, genetic, idiopathic and unclassified cardiomyopathy), sporadic dilated cardiomyopathy, X-linked Dilated Cardiomyopathy (XLDC), acute and chronic heart failure, right heart failure, left heart failure, biventricular heart failure, congenital heart defects, myocardiac fibrosis, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, systolic heart failure, diabetic heart failure and accumulation diseases.

### TGFβ Proteins

The disclosure provides compositions and methods directed to modulating the activity, including the expression, of LTBP4, which is a protein that interacts with TGFβ proteins. Modulation of the activity of any protein that interacts with LTBP4 is contemplated by the disclosure, and in various embodiments the TGFβ protein is selected from the group consisting of a growth and differentiation factor (GDF), activin, inhibin, and a bone morphogenetic protein. TGFβ proteins are known in the art and are discussed, for example and without limitation, in Schmierer et al. (Nature Reviews Molecular Cell Biology 8:970-982 (2007)], incorporated herein by reference. In addition, isoforms of TGFβ proteins are contemplated and include, without limitation, TGFβ 1, TGFβ 2, TGFβ 3, GDF 8, and GDF 11.

Practice of methods of the disclosure wherein a patient is administered one or more agent(s) and optionally additional agent(s) is expected to result in modulation of the activity of a TGFβ protein by at least about 1% relative to a patient not so treated. In further embodiments, the activity of a TGFβ protein in a patient that is administered one or more agent(s) and/or additional agent(s) is modulated by at least about 1% and up to any one of about 2%, about 5%, about 10% or about 15% TGFβ activity relative to a patient not so treated. In still further embodiments, the activity of a TGFβ protein in a patient that is administered one or more agent(s) and optionally additional agent(s) is modulated by at least about 10% and up to any one of about 15%, about 20%, about 25% or about 30% TGFβ activity relative to a patient not so treated. In further embodiments, the activity of a TGFβ protein in a patient that is administered one or more agent(s) and/or additional agent(s) is modulated by at least about 10% and up to any one of about 40%, about 50%, about 60%, about 70%, about 80%, about 90%, about 95%, about 99% or more TGFβ activity relative to a patient not so treated. In specific embodiments, the activity of a TGFβ protein in a patient that is administered one or more agent(s) and optionally additional agent(s) is modulated by at least about 1%, about 2%, about 5%, about 10%, about 20%, about 25%, about 30%, about 35%, about 40%, about 50%, about 60%, about 70%, about 80%, about 90%, about 95%, about 99% TGFβ activity or more relative to a patient not so treated. Protein activity may be quantitated by methods generally known to those of skill in the art.

### Additional (Second) Agents

In various embodiments of the disclosure it is contemplated that a second agent is administered with the agent that modulates LTBP4 activity by modulating the proteolysis of LTBP4. Nonlimiting examples of the second agent are a modulator of an inflammatory response, a promoter of muscle growth, a chemotherapeutic agent and a modulator of fibrosis. Further, the methods disclosed herein can, in various embodiments, encompass one or more of such agents, or one or more of such agents in composition with any other active agent(s).

### Chemotherapeutic Agents

Chemotherapeutic agents contemplated for use include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

### Modulators of Fibrosis

A “modulator of fibrosis” as used herein is synonymous with antifibrotic agent. The term “antifibrotic agent” refers to a chemical compound that has antifibrotic activity (i.e., prevents or reduces fibrosis) in mammals. This takes into account the abnormal formation of fibrous connective tissue, which is typically comprised of collagen. These compounds may have different mechanisms of action, some reducing the formation of collagen or another protein, others enhancing the catabolismor removal of collagen in the affected area of the body. All such compounds having activity in the reduction of the presence of fibrotic tissue are included herein, without regard to the particular mechanism of action by which each such drug functions. Antifibrotic agents useful in the methods and compositions of the disclosure include those described in U.S. Pat. No. 5,720,950, incorporated herein by reference. Additional antifibrotic agents contemplated by the disclosure include, but are not limited to, Type II interferon receptor agonists (e.g., interferon-gamma); pirfenidone and pirfenidone analogs; anti-angiogenic agents, such as VEGF antagonists, VEGF receptor antagonists, bFGF antagonists, bFGF receptor antagonists, TGFβ antagonists, TGFβ receptor antagonists; anti-inflammatory agents, IL-1 antagonists, such as IL-1Ra, angiotensin-converting-enzyme (ACE) inhibitors, angiotensin receptor blockers and aldosterone antagonists.

### Modulators of an Inflammatory Response

A modulator of an inflammatory response includes the following agents. In one embodiment of the disclosure, the modulator of an inflammatory response is a beta2-adrenergic receptor agonist (e.g., albuterol). The term beta2-adrenergic receptor agonist is used herein to define a class of drugs which act on the B2-adrenergic receptor, thereby causing smooth muscle relaxation resulting in dilation of bronchial passages, vasodilation in muscle and liver, relaxation of uterine muscle and release of insulin. In one embodiment, the beta2-adrenergic receptor agonist for use according to the disclosure is albuterol, an immunosuppressant drug that is widely used in inhalant form for asthmatics. Albuterol is thought to slow disease progression by suppressing the infiltration of macrophages and other immune cells that contribute to inflammatory tissue loss. Albuterol also appears to have some anabolic effects and promotes the growth of muscle tissue. Albuterol may also suppress protein degradation (possibly via calpain inhibition).

In DMD, the loss of dystrophin leads to breaks in muscle cell membrane, and destabilizes neuronal nitric oxide synthase (nNOS), a protein that normally generates nitric oxide (NO). It is thought that at least part of the muscle degeneration observed in DMD patients may result from the reduced production of muscle membrane-associated neuronal nitric oxide synthase. This reduction may lead to impaired regulation of the vasoconstrictor response and eventual muscle damage.

In one embodiment, modulators of an inflammatory response suitable for use in compositions of the disclosure are Nuclear Factor Kappa-B (NF-κB) inhibitors. NF—KB is a major transcription factor modulating cellular immune, inflammatory and proliferative responses. NF-κB functions in activated macrophages to promote inflammation and muscle necrosis and in skeletal muscle fibers to limit regeneration through the inhibition of muscle progenitor cells. The activation of this factor in DMD contributes to diseases pathology. Thus, NF-κB plays an important role in the progression of muscular dystrophy and the IKK/NF-κB signaling pathway is a potential therapeutic target for the treatment of a TGFβ-related disease. Inhibitors of NF-κB (for example, IRFI 042, a vitamin E analog) enhance muscle function, decrease serum creatine kinase (CK) level and muscle necrosis and enhance muscle regeneration. Furthermore, specific inhibition of NF-κB-mediated signaling by IKK has similar benefits.

In a further embodiment, the modulator of an inflammatory response is a tumor necrosis factor alpha antagonist. TNF-α is one of the key cytokines that triggers and sustains the inflammation response. In one specific embodiment of the disclosure, the modulator of an inflammatory response is the TNF-α antagonist infliximab.

TNF-α antagonists for use according to the disclosure include, in addition to infliximab (Remicade™), a chimeric monoclonal antibody comprising murine VK and VH domains and human constant Fc domains. The drug blocks the action of TNF-α by binding to it and preventing it from signaling the receptors for TNF-α on the surface of cells. Another TNF-α antagonist for use according to the disclosure is adalimumab (Humira™). Adalimumab is a fully human monoclonal antibody. Another TNF-α antagonist for use according to the disclosure is etanercept (Enbrel™). Etanercept is a dimeric fusion protein comprising soluble human TNF receptor linked to an Fc portion of an IgG1. It is a large molecule that binds to TNF-α and thereby blocks its action. Etanercept mimics the inhibitory effects of naturally occurring soluble TNF receptors, but as a fusion protein it has a greatly extended half-life in the bloodstream and therefore a more profound and long-lasting inhibitory effect.

Another TNF-α antagonist for use according to the disclosure is pentoxifylline (Trental™), chemical name 1-(5-oxohexyl)-3,7-dimethylxanthine. The usual dosage in controlled-release tablet form is one tablet (400 mg) three times a day with meals.

Dosing: Remicade is administered by intravenous infusion, typically at 2-month intervals. The recommended dose is 3 mg/kg given as an intravenous infusion followed with additional similar doses at 2 and 6 weeks after the first infusion, then every 8 weeks thereafter. For patients who have an incomplete response, consideration may be given to adjusting the dose up to 10 mg/kg or treating as often as every 4 weeks. Humira is marketed in both preloaded 0.8 ml (40 mg) syringes and also in preloaded pen devices, both injected subcutaneously, typically by the patient at home. Etanercept can be administered at a dose of 25 mg (twice weekly) or 50 mg (once weekly).

In another embodiment of the disclosure, the modulator of an inflammatory response is cyclosporin. Cyclosporin A, the main form of the drug, is a cyclic nonribosomal peptide of 11 amino acids produced by the fungus Tolypocladium inflatum. Cyclosporin is thought to bind to the cytosolic protein cyclophilin (immunophilin) of immunocompetent lymphocytes (especially T-lymphocytes). This complex of cyclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. It has also an effect on mitochondria, preventing the mitochondrial PT pore from opening, thus inhibiting cytochrome c release (a potent apoptotic stimulation factor). Cyclosporin may be administered at a dose of 1-10 mg/kg/day.

### A Promoter of Muscle Growth

In some embodiments of the disclosure, a therapeutically effective amount of a promoter of muscle growth is administered to a patient. Promoters of muscle growth contemplated by the disclosure include, but are not limited to, insulin-like growth factor-1 (IGF-1), Akt/protein kinase B, clenbuterol, creatine, decorin (see U.S. Patent Publication Number 20120058955), a steroid (for example and without limitation, a corticosteroid or a glucocorticoid steroid), testosterone and a myostatin antagonist.

### Myostatin Antagonist

Another class of promoters of muscle growth suitable for use in the combinations of the disclosure is myostatin antagonists. Myostatin, also known as growth/differentiation factor 8 (GDF-8) is a transforming growth factor-β (TGFβ) superfamily member involved in the regulation of skeletal muscle mass. Most members of the TGF-β-GDF family are widely expressed and are pleiotropic; however, myostatin is primarily expressed in skeletal muscle tissue where it negatively controls skeletal muscle growth. Myostatin is synthesized as an inactive preproprotein which is activated by proteolyic cleavage. The precurser protein is cleaved to produce an approximately 109-amino-acid COOH-terminal protein which, in the form of a homodimer of about 25 kDa, is the mature, active form. The mature dimer appears to circulate in the blood as an inactive latent complex bound to the propeptide. As used herein the term “myostatin antagonist” defines a class of agents that inhibits or blocks at least one activity of myostatin, or alternatively, blocks or reduces the expression of myostatin or its receptor (for example, by interference with the binding of myostatin to its receptor and/or blocking signal transduction resulting from the binding of myostatin to its receptor). Such agents therefore include agents which bind to myostatin itself or to its receptor.

Myostatin antagonists for use according to the disclosure include antibodies to GDF-8; antibodies to GDF-8 receptors; soluble GDF-8 receptors and fragments thereof (e.g., the ActRIIB fusion polypeptides as described in U.S. Patent Publication Number 2004/0223966, which is incorporated herein by reference in its entirety, including soluble ActRIIB receptors in which ActRIIB is joined to the Fc portion of an immunoglobulin); GDF-8 propeptide and modified forms thereof (e.g., as described in WO 2002/068650 or U.S. Pat. No. 7,202,210, including forms in which GDF-8 propeptide is joined to the Fc portion of an immunoglobulin and/or form in which GDF-8 is mutated at an aspartate (asp) residue, e.g., asp-99 in murine GDF-8 propeptide and asp-100 in human GDF-8 propeptide); a small molecule inhibitor of GDF-8; follistatin (e.g., as described in U.S. Pat. No. 6,004,937, incorporated herein by reference) or follistatin-domain-containing proteins (e.g., GASP-1 or other proteins as described in U.S. Pat. Nos. 7,192,717 and 7,572,763, each incorporated herein by reference); and modulators of metalloprotease activity that affect GDF-8 activation, as described in U.S. Patent Publication Number 2004/0138118, incorporated herein by reference.

Additional myostatin antagonists include myostatin antibodies which bind to and inhibit or neutralize myostatin (including the myostatin proprotein and/or mature protein, in monomeric or dimeric form). Myostatin antibodies are mammalian or non-mammalian derived antibodies, for example an IgNAR antibody derived from sharks, or humanized antibodies, or comprise a functional fragment derived from antibodies. Such antibodies are described, for example, in WO 2005/094446 and WO 2006/116269, the content of which is incorporated herein by reference. Myostatin antibodies also include those antibodies that bind to the myostatin proprotein and prevent cleavage into the mature active form. Additional antibody antagonists include the antibodies described in U.S. Pat. Nos. 6,096,506 and 6,468,535 (each of which is incorporated herein by reference). In some embodiments, the GDF-8 inhibitor is a monoclonal antibody or a fragment thereof that blocks GDF-8 binding to its receptor. Further embodiments include murine monoclonal antibody JA-16 (as described in U.S. Pat. No. 7,320,789 (ATCC Deposit No. PTA-4236); humanized derivatives thereof and fully human monoclonal anti-GDF-8 antibodies (e.g., Myo29, Myo28 and Myo22, ATCC Deposit Nos. PTA-4741, PTA-4740, and PTA-4739, respectively, or derivatives thereof) as described in U.S. Pat. No. 7,261,893 and incorporated herein by reference.

In still further embodiments, myostatin antagonists include soluble receptors which bind to myostatin and inhibit at least one activity thereof. The term “soluble receptor” herein includes truncated versions or fragments of the myostatin receptor that specifically bind myostatin thereby blocking or inhibiting myostatin signal transduction. Truncated versions of the myostatin receptor, for example, include the naturally occurring soluble domains, as well as variations produced by proteolysis of the N- or C-termini. The soluble domain includes all or part of the extracellular domain of the receptor, either alone or attached to additional peptides or other moieties. Because myostatin binds activin receptors (including the activin type IEB receptor (ActRHB) and activin type HA receptor (ActRHA)), activin receptors can form the basis of soluble receptor antagonists. Soluble receptor fusion proteins can also be used, including soluble receptor Fc (see U.S. Patent Publication Number 2004/0223966 and WO 2006/012627, both of which are incorporated herein by reference in their entireties).

Other myostatin antagonists based on the myostatin receptors are ALK-5 and/or ALK-7 inhibitors (see for example WO 2006/025988 and WO 2005/084699, each incorporated herein by reference). As a TGF-β cytokine, myostatin signals through a family of single transmembrane serine/threonine kinase receptors. These receptors can be divided in two classes, the type I or activin-like kinase (ALK) receptors and type II receptors. The ALK receptors are distinguished from the Type II receptors in that the ALK receptors (a) lack the serine/threonine-rich intracellular tail, (b) possess serine/threonine kinase domains that are highly homologous among Type I receptors, and (c) share a common sequence motif called the GS domain, consisting of a region rich in glycine and serine residues. The GS domain is at the amino terminal end of the intracellular kinase domain and is believed to be critical for activation by the Type II receptor. Several studies have shown that TGF-β signaling requires both the ALK (Type I) and Type II receptors. Specifically, the Type II receptor phosphorylates the GS domain of the Type 1 receptor for TGFβ ALK5, in the presence of TGFβ. The ALK5, in turn, phosphorylates the cytoplasmic proteins smad2 and smad3 at two carboxy terminal serines. Generally, it is believed that in many species, the Type II receptors regulate cell proliferation and the Type I receptors regulate matrix production. Various ALK5 receptor inhibitors have been described (see, for example, U.S. Pat. Nos. 6,465,493, 6,906,089, U.S. Patent Publication Numbers 2003/0166633, 2004/0063745 and 2004/0039198, the disclosures of which are incorporated herein by reference). Thus, the myostatin antagonists for use according to the disclosure may comprise the myostatin binding domain of an ALK5 and/or ALK7 receptor.

Other myostatin antagonists include soluble ligand antagonists that compete with myostatin for binding to myostatin receptors. The term “soluble ligand antagonist” herein refers to soluble peptides, polypeptides or peptidomimetics capable of non-productively binding the myostatin receptor(s) (e.g., the activin type HB receptor (ActRHA)) and thereby competitively blocking myostatin-receptor signal transduction. Soluble ligand antagonists include variants of myostatin, also referred to as “myostatin analogs” that have homology to, but not the activity of, myostatin. Such analogs include truncates (such as N- or C-terminal truncations, substitutions, deletions, and other alterations in the amino acid sequence, such as variants having non-amino acid substitutions).

Additional myostatin antagonists contemplated by the disclosure include inhibitory nucleic acids as described herein. These antagonists include antisense or sense polynucleotides comprising a single-stranded polynucleotide sequence (either RNA or DNA) capable of binding to target mRNA (sense) or DNA (antisense) sequences. Thus, RNA interference (RNAi) produced by the introduction of specific small interfering RNA (siRNA), may also be used to inhibit or eliminate the activity of myostatin.

In specific embodiments, myostatin antagonists include, but are not limited to, follistatin, the myostatin prodomain, growth and differentiation factor 11 (GDF-11) prodomain, prodomain fusion proteins, antagonistic antibodies or antibody fragments that bind to myostatin, antagonistic antibodies or antibody fragments that bind to the activin type IEB receptor, soluble activin type IHB receptor, soluble activin type IEB receptor fusion proteins, soluble myostatin analogs (soluble ligands), polynucleotides, small molecules, peptidomimetics, and myostatin binding agents. Other antagonists include the peptide immunogens described in U.S. Pat. No. 6,369,201 and WO 2001/05820 (each of which is incorporated herein by reference) and myostatin multimers and immunoconjugates capable of eliciting an immune response and thereby blocking myostatin activity. Other antagonists include the protein inhibitors of myostatin described in WO 2002/085306 (incorporated herein by reference), which include the truncated Activin type II receptor, the myostatin pro-domain, and follistatin. Other myostatin inhibitors include those released into culture from cells overexpressing myostatin (see WO 2000/43781), dominant negative myostatin proteins (see WO 2001/53350) including the protein encoded by the Piedmontese allele, and mature myostatin peptides having a C-terminal truncation at a position either at or between amino acid positions 335 to 375. The small peptides described in U.S. Patent Publication Number 2004/0181033 (incorporated herein by reference) that comprise the amino acid sequence WMCPP, are also suitable for use in the compositions of the disclosure.

### Vectors

An appropriate expression vector may be used to deliver exogenous nucleic acid to a recipient muscle cell in the methods of the disclosure. In order to achieve effective gene therapy, the expression vector must be designed for efficient cell uptake and gene product expression. Use of adenovirus or adeno-associated virus (AAV) based vectors for gene delivery have been described [Berkner, Current Topics in Microbiol, and Imunol. 158:39-66 (1992); Stratford-Perricaudet et al., Hum. Gene Ther. 1:241-256 (1990); Rosenfeld et al., Cell 8:143-144 (1992); Stratford-Perricaudet et al., J. Clin. Invest. 90:626-630 (1992)]. In one specific embodiment, the adeno-associated virus vector is AAV9. Specific methods for gene therapy useful in the context of the present disclosure depend largely upon the expression system employed; however, most methods involve insertion of coding sequence at an appropriate position within the expression vector, and subsequent delivery of the expression vector to the target muscle tissue for expression.

Additional delivery systems useful in the practice of the methods of the disclosure are discussed in U.S. Patent Publication Numbers 2012/0046345 and 2012/0039806, each of which is incorporated herein by reference in its entirety.

### Therapeutic Endpoints

In various aspects of the disclosure, use of the agent(s) and optional additional agent(s) as described herein provide one or more benefits related to specific therapeutic endpoints relative to a patient not receiving the agent(s) and/or additional agent(s).

In embodiments wherein the TGFβ-related disease is a muscle-related disease (e.g., a muscular dystrophy or cardiomyopathy), therapeutic endpoints include, but are not limited to, length of time until a patient is non-ambulatory, ambulatory capacity as measured by, for example and without limitation, six-minute-walk distance which has been shown to correlate with human LTBP4 SNPs [see, for example, Hersh et al., Am J Respir Crit Care Med. 173 (9): 977-84 (2006)], relative health of heart as determined by, e.g., echocardiography, magnetic resonance imaging (MRI), muscle mechanics, pulmonary function and/or amount of tissue fibrosis.

With respect to the length of time until a patient is non-ambulatory, it is contemplated that, in some embodiments, a patient that is administered one or more agent(s) and, optionally, additional agent(s) remains ambulatory at least 1 day and up to any of about 5, about 10, about 30, about 60 or about 90 days longer than a patient not so treated. In further embodiments, a patient that is administered one or more agent(s) and optional additional agent(s) remains ambulatory at least about 1 month and up to any of about 2, about 4, about 6, about 8, about 10 or about 12 months longer than a patient not so treated. Still further embodiments of the disclosure contemplate that a patient that is administered one or more agent(s) and, optionally, additional agent(s) remains ambulatory at least about 1 year and up to any of about 1.5, about 2, about 3, about 4, about 5, about 6, about 7, about 8, about 9, about 10 or more years longer than a patient not so treated.

In embodiments wherein the TGFβ-related disease is a cancer, therapeutic endpoints include but are not limited to a reduction in tumor volume (i.e., the size of the tumor measured by the amount of space taken up by it expressed in traditional units of volume (e.g., cubic centimeters) or as a percentage of the tissue or organ within which it is found (e.g., the tumor volume of prostate cancer is the percentage of the prostate taken up by the tumor)) and/or a reduction in metastasis. With respect to the reduction in tumor volume and/or a reduction in metastasis, it is contemplated that in some embodiments the tumor volume or amount of metastasis is reduced in a patient that is administered one or more agent(s) and, optionally, additional agent(s) by about 1% relative to a patient not so treated. In further embodiments, the tumor volume or amount of metastasis is reduced in a patient that is administered one or more agent(s) and, optionally, additional agent(s) by at least about 1% and up to any of about 2%, about 5%, about 10% or about 15% relative to a patient not so treated. In still further embodiments, the tumor volume or amount of metastasis is reduced in a patient that is administered one or more agent(s) and, optionally, additional agent(s) by at least about 10% and up to about 15%, about 20%, about 25% or about 30% relative to a patient not so treated. In further embodiments, the tumor volume or amount of metastasis is reduced in a patient that is administered one or more agent(s) and, optionally, additional agent(s) by at least about 10% and up to any of about 40%, about 50%, about 60%, about 70%, about 80%, about 90%, about 95%, about 99% or more relative to a patient not so treated. In specific embodiments, the tumor volume or amount of metastasis is reduced in a patient that is administered one or more agent(s) and, optionally, additional agent(s) by at least about 1%, about 2%, about 5%, about 10%, about 20%, about 25%, about 30%, about 35%, about 40%, about 50%, about 60%, about 70%, about 80%, about 90%, about 95%, about 99% or more relative to a patient not so treated. Methods of measuring tumor volume as well as amount of metastasis are known in the art.

In embodiments wherein the TGFβ-related disease is a viral disease, therapeutic endopoints relate to the viral load in the patient. Methods of determining viral load are well known in the art and can be quantitated using methods such as polymerase chain reaction (PCR), reverse-transcriptase PCR (RT-PCR), probe-specific amplification or by the branched DNA (bDNA) method. In various embodiments, the viral load of a patient being administered one or more agent(s) and, optionally, additional agent(s) of the disclosure is reduced by at least about 1% and up to any of about 5%, about 10%, about 20%, about 30%, about 40% or about 50% relative to a patient not so treated. In further embodiments, the viral load of a patient being administered one or more agent(s) and, optionally, additional agent(s) of the disclosure is reduced by at least about 10% and up to any of about 20%, about 50%, about 70%, about 80%, about 90%, about 99% or more relative to a patient not so treated. In specific embodiments, the viral load of a patient being administered one or more agent(s) and/or additional agent(s) of the disclosure is reduced by at least about 1%, about 2%, about 5%, about 10%, about 20%, about 25%, about 30%, about 35%, about 40%, about 50%, about 60%, about 70%, about 80%, about 90%, about 95%, about 99% or more relative to a patient not so treated.

In general, a therapeutic endpoint achieved by practice of the methods of the disclosure is a reduction in the amount of fibrosis in a patient being administered one or more agent(s) and, optionally, additional agent(s) of the disclosure. Relative amounts of fibrosis in a patient can be quantitated by tissue biopsy and subsequent histology, e.g., by quantifying Evans blue dye uptake as a measure of myofiber or cellular damage [Heydemann et al., Neuromuscular Disorders 15 (9-10): 601-9 (2005)], and/or quantitation of hydroxyproline content as described previously [Swaggart et al., Physiol Genomics 43:24-31 (2011)]. In various embodiments, the amount of fibrosis in a patient being administered one or more agent(s) and, optionally, additional agent(s) of the disclosure is reduced by at least about 1% and up to any of about 5%, about 10%, about 20%, about 30%, about 40% or about 50% relative to a patient not so treated. In further embodiments, the amount of fibrosis in a patient being administered one or more agent(s) and, optionally, additional agent(s) of the disclosure is reduced by at least about 10% and up to about 20%, about 50%, about 70%, about 80%, about 90%, about 99% or more relative to a patient not so treated. In specific embodiments, the amount of fibrosis in a patient being administered one or more agent(s) and/or additional agent(s) of the disclosure is reduced by at least about 1%, about 2%, about 5%, about 10%, about 20%, about 25%, about 30%, about 35%, about 40%, about 50%, about 60%, about 70%, about 80%, about 90%, about 95%, about 99% or more relative to a patient not so treated.

The amount of fibrosis in a patient can be routinely determined by one of ordinary skill in the art. For example, and without limitation, the amount of fibrosis can be determined by taking a muscle biopsy from a patient, sectioning the muscle onto slides and assessing the amount of fibrosis as revealed by staining techniques known in the art (e.g., Hematoxylin and Eosin (H&E) staining and/or Masson's trichrome staining). Alternatively, or in addition, the amount of fibrosis can be determined in vivo by using magnetic resonance imaging (MRI).

### Dosing/Administration/Kits

A particular administration regimen for a particular subject will depend, in part, upon the agent and optional additional agent used, the amount of the agent and optional additional agent administered, the route of administration, the particular ailment being treated, and the cause and extent of any side effects. The amount of agent and optional additional agent administered to a subject (e.g., a mammal, such as a human) is sufficient to effect the desired response. Dosage typically depends upon a variety of factors, including the particular agent and/or additional agent employed, the age and body weight of the subject, as well as the existence and severity of any disease or disorder in the subject. The size of the dose also will be determined by the route, timing, and frequency of administration. Accordingly, the clinician may titer the dosage and modify the route of administration to obtain optimal therapeutic effect, and conventional range-finding techniques are known to those of ordinary skill in the art. Purely by way of illustration, in some embodiments, the method comprises administering, e.g., from about 0.1 μg/kg up to about 100 mg/kg or more, depending on the factors mentioned above. In other embodiments, the dosage may range from 1 μg/kg up to about 75 mg/kg; or 5 μg/kg up to about 50 mg/kg; or 10 μg/kg up to about 20 mg/kg. In certain embodiments, the dose comprises about 0.5 mg/kg to about 20 mg/kg (e.g., about 1 mg/kg, 1.5 mg/kg, 2 mg/kg, 2.3 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 8 mg/kg, 9 mg/kg, or 10 mg/kg) of agent and optional additional agent. In embodiments in which an agent and additional agent are administered, the above dosages are contemplated to represent the amount of each agent administered, or in further embodiments the dosage represents the total dosage administered. Given the chronic nature of many TGFβ-related disorders, it is envisioned that a subject will receive the agent and/or additional agent over a treatment course lasting weeks, months, or years, and may require one or more doses daily or weekly. Dosages are also contemplated for once daily, twice daily (BID) or three times daily (TID) dosing. A unit dose may be formulated in either capsule or tablet form. In other embodiments, the agent and optional additional agent is administered to treat an acute condition (e.g., acute muscle injury or acute myocardial injury) for a relatively short treatment period, e.g., one to 14 days.

Suitable methods of administering a physiologically-acceptable composition, such as a pharmaceutical composition comprising an agent and optional additional agent described herein, are well known in the art. Although more than one route can be used to administer an agent and/or additional agent, a particular route can provide a more immediate and more effective avenue than another route. Depending on the circumstances, a pharmaceutical composition is applied or instilled into body cavities, absorbed through the skin or mucous membranes, ingested, inhaled, and/or introduced into circulation. In some embodiments, a composition comprising an agent and/or additional agent is administered intravenously, intraarterially, or intraperitoneally to introduce an agent and optional additional agent into circulation. Non-intravenous administration also is appropriate, particularly with respect to low molecular weight therapeutics. In certain circumstances, it is desirable to deliver a pharmaceutical composition comprising the agent and/or additional agent orally, topically, sublingually, vaginally, rectally; through injection by intracerebral (intra-parenchymal), intracerebroventricular, intramuscular, intra-ocular, intraportal, intralesional, intramedullary, intrathecal, intraventricular, transdermal, subcutaneous, intranasal, urethral, or enteral means; by sustained release systems; or by implantation devices. If desired, the agent and/or additional agent is administered regionally via intraarterial or intravenous administration to a region of interest, e.g., via the femoral artery for delivery to the leg. In one embodiment, the composition is administered via implantation of a membrane, sponge, or another appropriate material within or upon which the desired agent and optional additional agent has been absorbed or encapsulated. Where an implantation device is used, the device in one aspect is implanted into any suitable tissue, and delivery of the desired agent and/or additional agent is, in various embodiments, effected via diffusion, time-release bolus, or continuous administration. In other embodiments, the agent and optional additional agent is administered directly to exposed tissue during surgical procedures or treatment of injury, or is administered via transfusion of blood products. Therapeutic delivery approaches are well known to the skilled artisan, some of which are further described, for example, in U.S. Pat. No. 5,399,363.

In some embodiments facilitating administration, the agent and optional additional agent in one embodiment is formulated into a physiologically-acceptable composition comprising a carrier (i.e., vehicle, adjuvant, buffer, or diluent). The particular carrier employed is limited only by chemico-physical considerations, such as solubility and lack of reactivity with the agent and/or additional agent, by the route of administration, and by the requirement of compatibility with the recipient organism. Physiologically acceptable carriers are well known in the art. Illustrative pharmaceutical forms suitable for injectable use include, without limitation, sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986), incorporated herein by reference).

A pharmaceutical composition comprising an agent and optional additional agent as provided herein is optionally placed within containers/kits, along with packaging material that provides instructions regarding the use of such pharmaceutical compositions. Generally, such instructions include a tangible expression describing the reagent concentration, as well as, in certain embodiments, relative amounts of excipient ingredients or diluents that may be necessary to reconstitute the pharmaceutical composition.

The disclosure thus includes administering to a subject one or more agent(s), in combination with one or more additional agent(s), each being administered according to a regimen suitable for that medicament. Administration strategies include concurrent administration (i.e., substantially simultaneous administration) and non-concurrent administration (i.e., administration at different times, in any order, whether overlapping or not) of the agent and one or more additional agents(s). It will be appreciated that different components are optionally administered in the same or in separate compositions, and by the same or different routes of administration.

All publications, patents and patent applications cited in this specification are herein incorporated by reference as if each individual publication or patent application were specifically and individually indicated to be incorporated by reference. In addition, the entire document is intended to be related as a unified disclosure, and it should be understood that all combinations of features described herein are contemplated, even if the combination of features are not found together in the same sentence, or paragraph, or section of this document. For example, where protein therapy is described, embodiments involving polynucleotide therapy (using polynucleotides/vectors that encode the protein) are specifically contemplated, and the reverse also is true. With respect to elements described as one or more members of a set, it should be understood that all combinations within the set are contemplated.

### EXAMPLES

### Example 1

### Structure-Function Relationship Between the Proline-Rich Region of LTBP4 and Proteolytic Susceptibility

The data provided in this Example show that the proline-rich region of LTBP4 contributes to its proteolytic susceptibility.

LTBP4 binds to TGFβ in the extracellular matrix (ECM), where it serves as a readily available TGFβ storage site. A 36-nucleotide deletion was identified in the proline-rich domain of murine LTBP4 that associates with enhanced pathogenic features of muscular dystrophy in mice. This region in murine LTBP4 is associated with variable susceptibility to proteolysis. Sequence comparison analysis between LTBP4 from mouse and humans reveals an even larger deletion in the proline-rich region of human LTBP4. Thus, consistent with the murine deletion being associated with pathogenic features and variable proteolysis [see Heydemann et al., J Clin Invest. 119 (12): 3703-12 (2009)], it was contemplated that the larger deletion of the proline-rich region of human LTBP4 was associated with enhanced susceptibility to proteolytic cleavage.

To investigate this possibility, a portion of the human LTBP4 coding region was ligated into an expression vector to express the proline-rich region. The TP fragment (amino acids 483-565 of the human LTBP4 protein (SEQ ID NO: 1)) was expressed and migrated as a 3.5 KDa protein although its predicted molecular mass is 8.9 KDa. A second fragment, TP2E fragment (amino acids 357-586 of the human LTBP4 protein (SEQ ID NO: 1)) was also expressed. Its predicted molecular mass is 24.5 KDa, yet it electrophoretically migrated as a 31 KDa protein. TP2E included the two EGF-like domains that flank the proline-rich region of LTBP4 along with the amino terminal 8-cysteine rich region immediately amino-terminal of the proline-rich region. Murine TP2E and TP each contained an additional 44 amino acids compared to the human sequences, reflecting the larger proline-rich region. The murine TP2E electrophoretically migrates as a 35 KDa protein while its calculated molecular mass is 30.58 KDa.

### Susceptibility to Proteolysis In Vitro

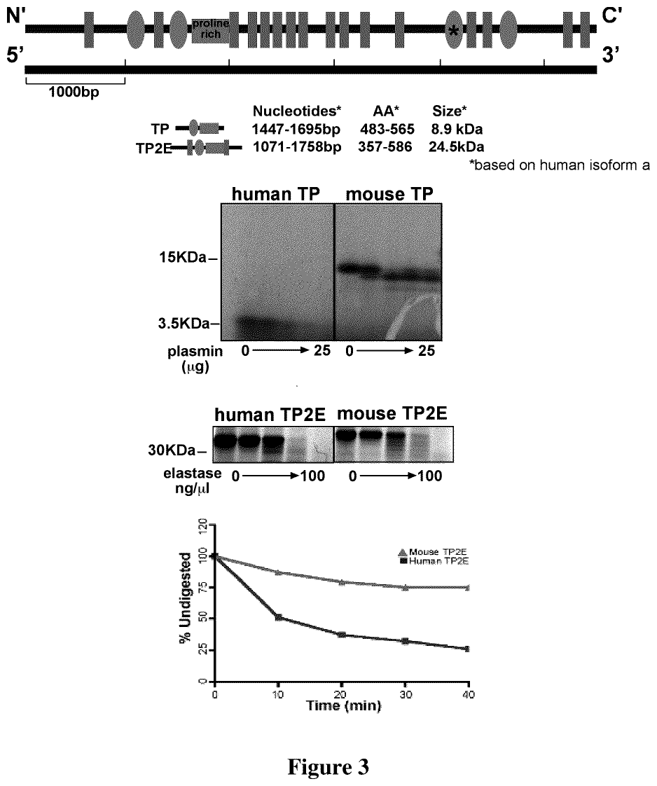

Human and mouse TP2E fragments were expressed in vitro using a transcription-translation coupled assay (Promega TnT® Quick Coupled in vitro Transcription/Translation System) and the expressed fragments were labeled using 35S-Cysteine. Dose-response and time course experiments were performed with elastase and plasmin, which are both serine proteases that cleave LTBP4, to determine the differential digestion of the human and mouse TP2E fragments. Data from these experiments showed that the human TP2E fragment is more readily cleaved than the mouse LTBP4 sequence (FIG. 3).

### Effects of Smaller Fragments of LTBP4 on TGFβ Signaling

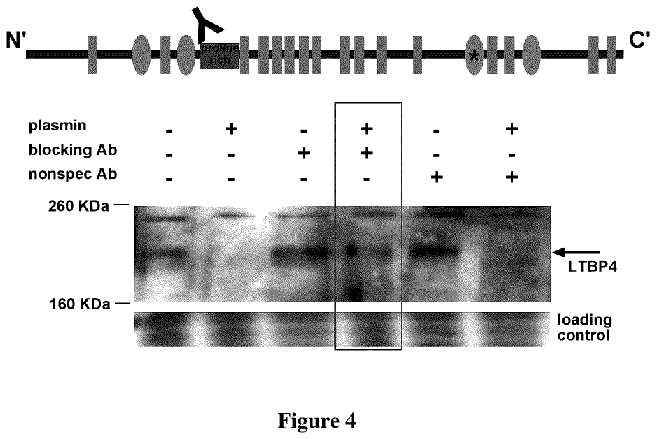

An antibody to the proline-rich region of human LTBP4 was generated to inhibit LTBP4 cleavage. This antibody was tested and confirmed to recognize and bind to the full-length human LTBP4 by immunoblot. Conditions were then optimized for the digestion of full-length human LTBP4 by plasmin, and inhibition of proteolysis using the antibody was tested. The data in FIG. 4 show that the anti-LTBP4 antibody specifically inhibited the protein digestion compared to a nonrelated antibody raised in the same species (FIG. 4).

### Example 2

### Effect of Human LTBP4 Expression on Muscle and Cardiac Phenotypes

LTBP4 plays a critical role in TGFβ secretion and activation in cardiac muscle, skeletal muscle and lung. Human LTBP4 has a larger deletion in the proline-rich region compared to a mutant murine LTBP4, with wild-type murine LTBP4 used as a reference. Thus, it is contemplated that human LTBP4 is associated with increased pathogenic TGFβ signaling and, therefore, will be associated with more severe disease in mice with muscular dystrophy.

### Transgenic Mice Expressing Human LTBP4

A mouse harboring the human LTBP4 gene was generated according to standard protocols [see, e.g., Heintz, Nat Rev Neurosci. 2 (12): 861-70 (2001)]. A bacterial artificial chromosome (BAC) that included the complete human LTBP4 gene; the BAC transgenic-positive (Tg+) mice are referred to as hLTBP4 Tg+. To generate the hLTBP4 Tg+ mice, a single, unmodified BAC clone (clone number CDT-2166J9) was used to inject a fertilized oocyte using conventional methodology [see, e.g., Heintz, Nat Rev Neurosci. 2 (12): 861-70 (2001)]. The human sequence of this BAC (Genbank accession number AC010412.9; SEQ ID NO: 7) contains 155085 bp from chromosome 19. The LTBP4 gene spans from 19891 to 57891 bp of this clone. Eleven founder lines were evaluated by PCR and found to contain the full-length human LTBP4, including promoter regions. Six lines were chosen for breeding to ensure that these mice were passing the BAC in their germline. By RT-PCR, it was determined that the human LTBP4 mRNA was expressed in cardiac and skeletal muscle of the transgenic mice. At present, there is no antibody that distinguishes human LTBP4 from mouse LTBP4; human and mouse LTBP4 are 98% similar. Overall, LTBP4 expression may be slightly elevated in hLTBP4 Tg+ mice compared to littermate controls. hLTBP4 Tg+ mice are outwardly normal and breed normally. Histological examination showed grossly normal histology in brain, kidney, lung, heart and muscle. Interestingly, hLTBP4 Tg+ skeletal muscle fibers were significantly larger than littermate control transgene negative mice. It is contemplated that even modest overexpression of LTBP4 may be sufficient to bind other TGFβ superfamily members such as myostatin, and sequestration of myostatin would inhibit myostatin activity and would be expected to result in larger muscle fibers.

The hLTBP4 Tg+ animal will be bred to the mouse mdx model of Duchenne Muscular Dystrophy and the phenotype and TGFβ signaling capacity will be assessed. Ten mice of each genotype (hLTBP4 Tg+/mdx, mdx, hLTBP4+ and WT) will be generated. Basic neuromuscular function will be evaluated using SHIRPA protocols. SHIRPA is a combination of neurological tests that assess neuromuscular function [Rafael et al., Mamm Genome. 11 (9): 725-8 (2000)]. For example and without limitation, grip strength, running capacity, wire maneuver and rotorod are basic tests that will be used to assess muscle function. In addition, cardiac function will be assessed using echocardiography, and histology will be performed to evaluate fibrosis and membrane permeability using Evans blue dye uptake. All analyses will be conducted on male mice at 8 weeks of age. A cohort of mice will also be aged to examine the effect on mice at a later time point(s). Fibroblasts will also be isolated from these mice to determine their level of SMAD signaling using methods as previously described [Heydemann et al., J Clin Invest. 119 (12): 3703-12 (2009)]. It is expected that insertion of the human LTBP4 will result in increased SMAD signaling and enhancement of the mdx phenotype.

### Example 3

### LTBP4 Peptides and Antibody Generation

Antibodies were generated using multiple different peptides including the mouse and human LTBP4 sequences (see Table 1, below). Each of the peptides in Table 1 cross reacts to the human protein as determined by immunoblotting. A longer LTBP4 peptide, FLPTHRLEPRPEPRPDPRPGPELPLPSIPAWTGPEIPESGPSS (SEQ ID NO: 6), is also contemplated for use according to the disclosure. Humanized monoclonal antibodies directed against LTBP4 will also be generated.

TABLE 1

| | | | | West- | Results |
|---|---|---|---|---|---|
| | | | | ern | on |
| | | | | Blot | Proteo- |
| Antibody | | Peptide | Antigen | Activ- | lysis |
| Name | Species | Used | Source | ity | |
| mLTBP4d36- | chicken | EPRPRPEPRP | Mouse - | +++ | NA |
| 829 | | QPESQPWP | D2 | | |
| | | (SEQ ID | | | |
| | | NO: 2) | | | |
| mLTBP4d36- | chicken | EPRPRPEPRP | Mouse - | ++ | NA |
| 830 | | QPESQPWP | D2 | | |
| | | (SEQ ID | | | |
| | | NO: 2) | | | |
| hLTBP4pr- | chicken | EPRPEPRPDP | Human | ++++ | positive |
| 831 | | RPGPELP | | | |
| | | (SEQ ID | | | |
| | | NO: 3) | | | |
| hLTBP4pr- | chicken | EPRPEPRPDP | Human | ++ | NA |
| 832 | | RPGPELP | | | |
| | | (SEQ ID | | | |
| | | NO: 3) | | | |
| mLTBP4ins- | rabbit | ESQPRPESRP | Mouse - | ++ | NA |
| 24226 | | RPESQPWP | 129 | | |
| | | (SEQ ID | | | |
| | | NO: 4) | | | |
| mLTBP4ins- | rabbit | ESQPRPESRP | Mouse - | ++ | NA |
| 24226 | | RPESQPWP | 129 | | |
| | | (SEQ ID | | | |
| | | NO: 4) | | | |
| hLTBP4 | rabbit | EPRPEPRPDP | Human | NA | NA |
| (511-530) | | RPGPELPLP | | | |
| 28200 | | (SEQ ID | | | |
| | | NO: 5) | | | |
| hLTBP4 | rabbit | EPRPEPRPDP | Human | NA | NA |
| (511-530) | | RPGPELPLP | | | |
| 28199 | | (SEQ ID | | | |
| | | NO: 5) | | | |

LTBP4 peptides used for antibody generation. ″Species″ indicates antibody source.

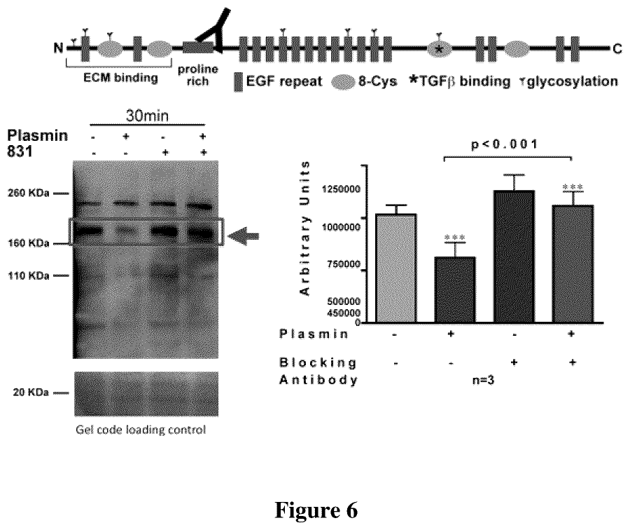

Table 1 shows that each antibody recognized a protein the size of human LTBP4, as determined by immunoblot. The data in the table also indicates that the antibody raised against the human sequence (SEQ ID NO: 3) protects LTBP4 against proteolysis in vitro (FIG. 6, described below) and, given the cross reactivity, the other anti-hLTBP4 antibodies are also expected to protect hLTBP4 from proteolysis. Enzyme-linked immunosorbent assays (ELISA) will also be performed to compare the relative affinity of antibodies to each peptide using serum and purified antibodies.

Proteolysis of LTBP4 can be Inhibited with LTBP4 Antibodies

It was contemplated that the insertion/deletion polymorphism in murine Ltbp4 discussed hereinabove indicated that the proline-rich region is important since the presence or absence of 12 additional amino acids in this region explains its ability to reduce membrane leak and suppress fibrosis, two activities that were attributed to LTBP4's ability to sequester TGFβ. This position is consistent with the differential sensitivity to proteolysis of the various forms of LTBP4 and the associated TGFβ activity in the form of nuclear pSMAD [Heydemann et al., J Clin Invest. 119:3703-12 (2009)].

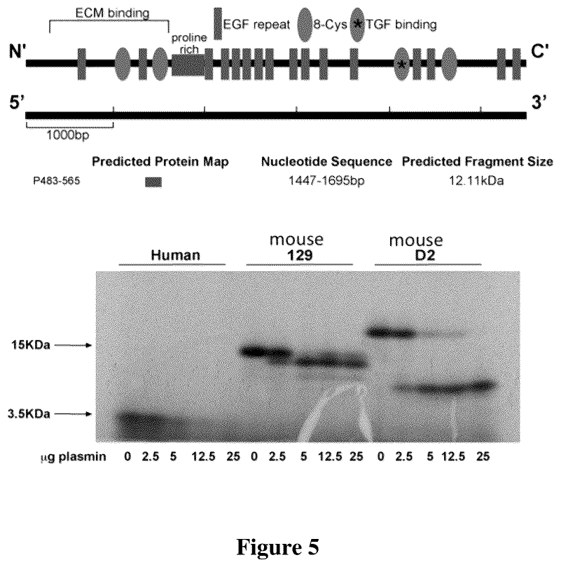

To demonstrate that the proline-rich region of human LTBP4 was susceptible to proteolysis, protein domains were expressed using in vitro transcription and translation according to methods as previously described [Heydemann et al., J Clin Invest. 119 (12): 3703-12 (2009)]. By design, only the carboxy-terminus of these expressed proteins was labeled. The expressed fragments were exposed to plasmin. Murine LTBP4 with the 12-amino-acid insertion was largely resistant to proteolysis while the murine LTBP4 deleted for the 12 amino acids was readily degraded (FIG. 5, middle and right lanes). The human LTBP4 was most readily degraded (FIG. 5, left lanes). Similar results were obtained with elastase. It is contemplated that this region (i.e., the region included in the TP and TP2E sequences) is a general serine protease target. Antibodies were generated that were directed at the proline-rich region and it was found that these antibodies inhibited LTBP4 cleavage in vitro (FIG. 6). A nonspecific antibody generated from the same species showed no blocking effect. Additional anti-LTBP4 antibodies have been generated, and Fab fragments will be purified and tested because these fragments are expected to be more useful for in vivo delivery.

Full-length LTBP4 protein, produced from cultured cells, is also susceptible to plasmin proteolysis (FIG. 6). With muscle injury, such as the injury that occurs in DMD, release of proteases into the extracellular matrix is expected to result in LTBP4 cleavage. The sources of these proteases in vivo may be inflammatory cells, fibroblasts or the myofibers. Increased LTBP4 cleavage was shown to correlate with increased fibrosis, increased muscle membrane leak, increased muscle weakness and increased TGFβ signaling [Heydemann et al., J Clin Invest. 119 (12): 3703-12 (2009)]. Reduction of TGFβ signaling was shown to improve outcome in muscular dystrophy [Cohn et al., Nat Med. 13 (2): 204-10 (2007); Goldstein et al., Hum Mol Genet. 20 (5): 894-904 (2011)].

This example demonstrates that proteolysis of the proline-rich region of LTBP4 can be inhibited by antibodies provided herein.

### Example 4

### Transgenic Mice Harboring Human Ltbp4

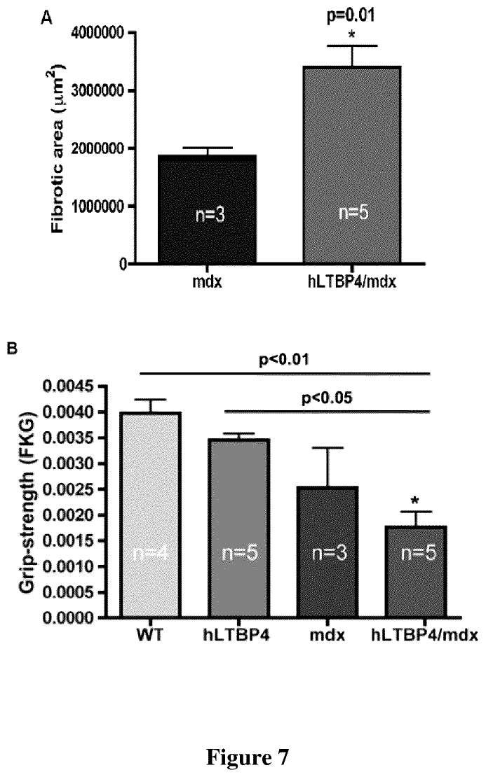

A human bacterial artificial chromosome (BAC) carrying the full length human LTBP4 gene was isolated and characterized. This BAC was injected into mice and several lines of transgenic mice were characterized. Human LTBP4 (hLTBP4-BAC) transgenic mice were bred to mdx mice. The human LTBP4 BAC in the normal background resulted in larger myofiber diameter, a sign of hypertrophy. When the human LTBP4 BAC was in the mdx background, it resulted in enhanced fibrosis in skeletal and cardiac muscle as well as reduced grip strength, relative to control mice that did not carry the transgene (FIG. 7). This supports the observation that the human LTBP4 sequence, with its larger deletion in the proline-rich region, enhances the muscular dystrophy phenotype. These mice will be further used to test whether antibodies directed against human LTBP4 can reduce muscular dystrophy fibrosis and muscle membrane leakage.

### Example 5

### In Vivo Studies

Short-term studies are conducted in dystrophic mice (i.e., mdx and limb girdle muscular dystrophy (LGMD)) to determine safety and efficacy of inhibiting LTBP4 cleavage in vivo. Animals are treated from 3 weeks to 8 weeks of age with antibody injections, three times weekly, delivered via intraperitoneal injection. Dose responsiveness is determined. Echocardiography, plethysmography, muscle harvest and ex vivo muscle mechanics are conducted on treated animals and controls. Target tissues are studied, including heart, diaphragm, quadriceps, gluteus/hamstrings, gastrocnemius/soleus, triceps and abdominal muscles, according to previously identified protocols [Heydemann et al., Neuromuscul Disord. 15:601-9 (2005); Heydemann, et al., J Clin Invest. 119:3703-12 (2009); Swaggart et al., Physiol Genomics. 43:24-31 (2011)]. TGFβ signaling is also determined.

Long-term studies are conducted in dystrophic mice to determine the safety and efficacy of the treatment. Once dosing has been determined, cohorts of mice are treated from 3 weeks until 1 year of age. A similar analysis of efficacy are undertaken, as discussed above (i.e., echocardiography, plethysmography, muscle harvest and ex vivo muscle mechanics). Analysis of other organs, including lung, colon, kidney, brain, and other tissues, is included. Mice that are null for LTBP4 develop cardiomyopathy, pulmonary fibrosis and colon cancer. Because LTBP4 is not ablated in these studies, these cardiomyopathy, pulmonary fibrosis and colon cancer defects are not expected, consistent with the results of the genetic studies described above. Nonetheless, off-target tissues are also analyzed.

The studies described above are expected to show that inhibition of LTBP4 cleavage in vivo results in decreased TGFβ signaling, which is further expected to lead to a decrease in membrane permeability as well as a decrease in fibrosis in the muscles of dystrophic mice. These results will be evidenced by an improvement or lack of decline in therapeutic endpoints as described herein, thereby establishing that blockage of LTBP4 proteolysis is a robust therapeutic in the treatment of TGFβ superfamily protein-related diseases.

### Example 6

LTBP4 Interacts with Myostatin In Vitro

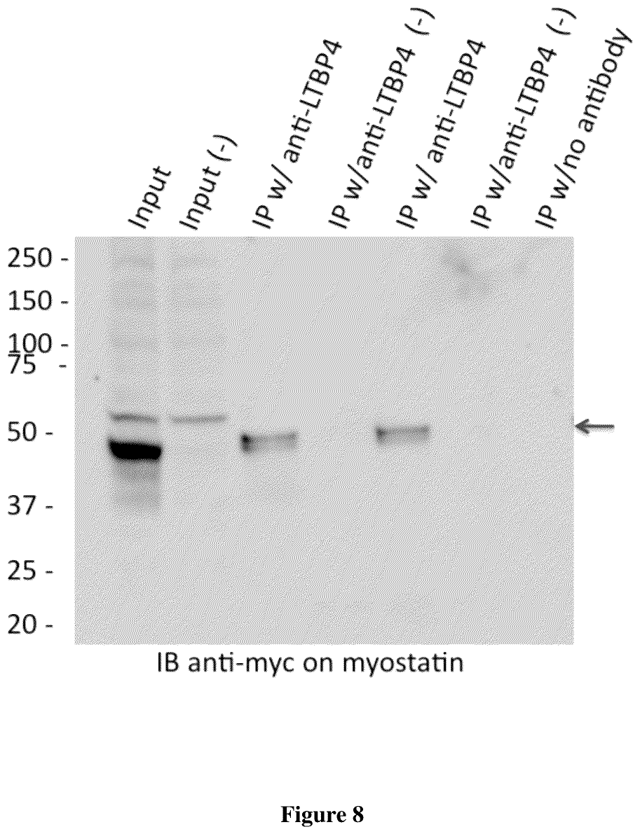

The ability of LTBP4 to directly interact with myostatin, a TGF-β superfamily member, was also investigated. The methods used to investigate the interaction were as follows. Full length LTBP4 was cloned into an expression vector (pcDNA3.1, Life Technologies (Invitrogen), Grand Island, NY) and the Xpress epitope tag (Life Technologies (Invitrogen), Grand Island, NY) was added to its 5′ end/amino terminus. Full-length myostatin, encoding the propeptide and mature regions, was tagged at its 3′ end/carboxy terminus with the myc epitope tag. Both plasmids were introduced into HEK293 (Human Embryonic Kidney 293) cells. The cells were lysed and the proteins were blotted or immunoprecipitated with either antibody 28200 or, in a separate experiment, antibody 28199 (see Table 1). Both of these rabbit polyclonal antibodies are directed at the LTBP4 proline-rich region. The immunoprecipitated material was then blotted with the anti-myc antibody, showing that myostatin associates with LTBP4 (FIG. 8).

This Example shows that LTBP4 is able to directly interact with myostatin. The results indicate that, by inhibiting the proteolysis of LTBP4 according to the present disclosure, one can sequester myostatin and prevent its activation and resultant downstream signaling. Because myostatin is a known negative regulator of muscle growth, the inhibition of myostatin signaling is expected to result in increased muscle growth and increased muscle strength.

### Example 7

### Expression of Human LTBP4 in Mice Leads to Enhanced Damage After Cardiotoxin Injury

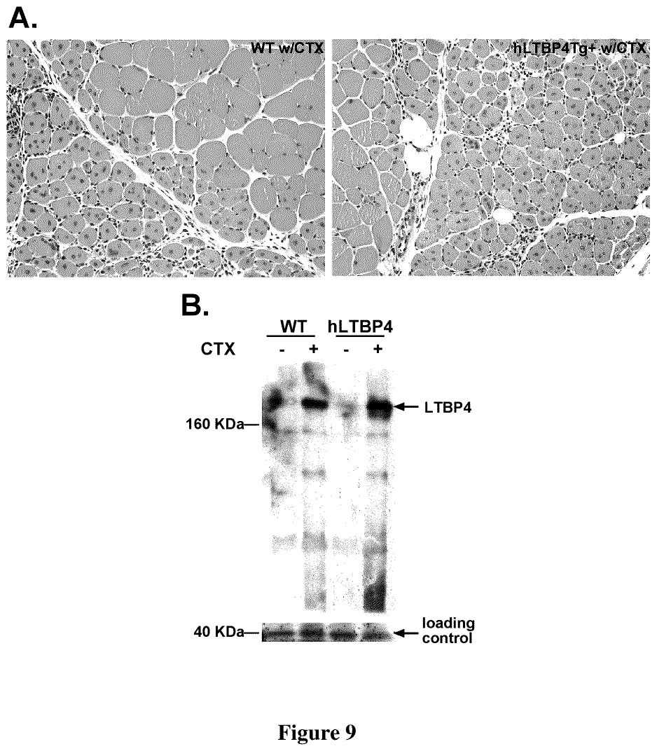

Mice were generated to express the human LTBP4 gene on a bacterial artificial chromosome, and these transgenic mice were referred to as hLTBP4 Tg+ mice. The human LTBP4 protein is more readily proteolyzed because of its shorter proline-rich region. This increased proteolysis leads to enhanced damage in muscle due to increased TGFβ release. Cardiotoxin was injected into the tibialis anterior muscle of normal (WT w/CTX) and hLTBP4 transgenic mice (hLTBP4 Tg+w/CTX). Transgenic mice displayed enhanced injury after cardiotoxin injury seen as greater inflammatory mononuclear cell infiltrate and fibrosis and fat deposition into the injured muscle (FIG. 9A), similar to what is seen in muscular dystrophy.

Normal (WT) and hLTBP4 muscle were injected with cardiotoxin to induce injury. Immunoblotting with an anti-LTBP4 antibody showed increased levels of LTBP4 protein induced by injury in both normal and in hLTBP4 transgenic muscle (FIG. 9B). hLTBP4 muscle was also found to be associated with increased TGFβ signaling seen as nuclear localized phosphorylated SMAD.

The results showed that expression of human LTBP4 protein in muscle leads to enhanced muscle damage following cardiotoxin injury.

### Example 8

### Anti-LTBP4 Antibodies Mitigate Muscle Injury In Vivo

To test whether anti-LTBP4 antibody mitigated skeletal muscle injury in muscular dystrophy, experiments were carried out using hLTBP4/mdx mice. Cardiotoxin, which is known to cause necrosis of skeletal muscle cells, was injected into the tibialis anterior muscle to induce enhanced injury. This injury model resolves within 2 weeks because a low-volume injection of 10 μl is used. hLTBP4/mdx mice (8 weeks of age) were pretreated on day 0 with either (i) PBS or (ii) antibody to LTBP4-831 antibody at 5 mg/Kg intraperitoneally. On day 1, cardiotoxin was injected into the tibialis anterior muscle. LTBP4-831 antibody was injected on days 1, 3, and 5, each time delivering a 5 mg/Kg dose intraperitoneally. Mice were sacrificed on day 7 and tibialis anterior muscle was harvested for study. The experimental design of sacrificing the mice on day 7 was used because the LTBP4-831 antibody is a chicken antibody that was expected to be recognized as foreign after 2-3 weeks.

Following harvest, the muscle was processed for analysis by snap-freezing in liquid nitrogen-cooled isopentane. The frozen muscle was sectioned and the sections were subjected to hematoxylin and eosin (H&E) staining.

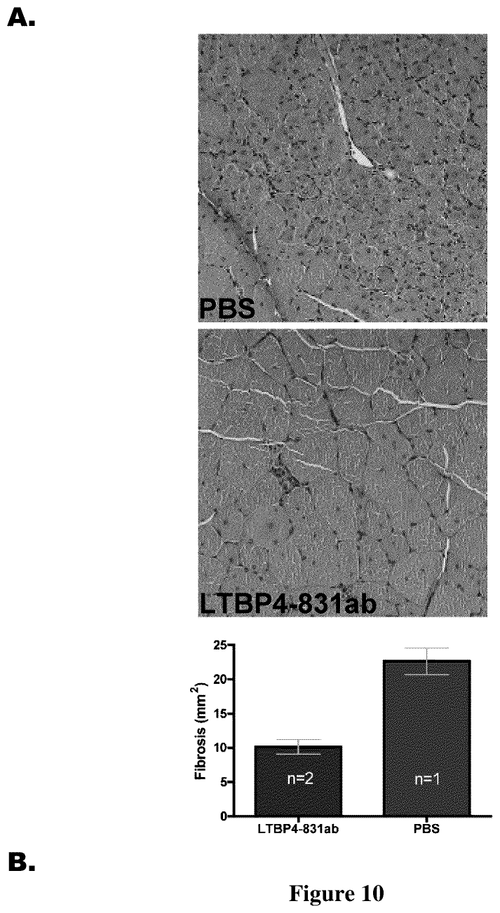

Results of the experiment showed that, compared to PBS-injected mice, LTBP4-831 antibody-treated mice showed reduced central nucleation and reduced fibrosis (FIGS. 10A and 10B). Centralized nuclei are indicative of newly formed (i.e., regenerating) myofibers, and reduced central nucleation in the muscle of animals that were administered LTBP4-831 antibody provides evidence that the antibody mitigated muscle injury in the mice.

### Example 9

Increased Inflammatory Infiltrate in hLTBP4/mdx Mice Compared to mdx Mice

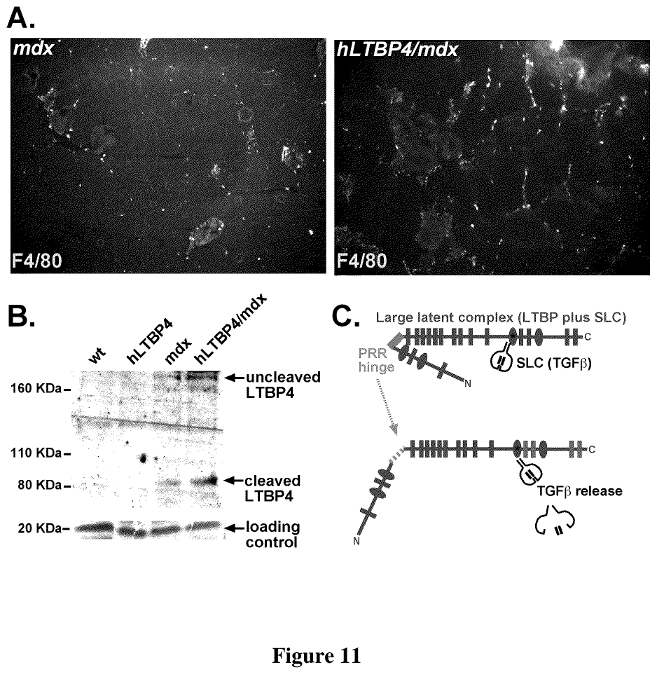

Quadriceps muscles obtained from both mdx and hLTPB4/mdx mice were stained with F4/80 antibodies that recognize and bind to activated macrophages (shown as speckles throughout the muscle). The immunofluorescent staining showed an increase in activated macrophages in hLTBP4/mdx muscle compared to mdx muscle (FIG. 11A). hLTBP4/mdx muscle showed an increase in cleaved LTBP4 protein compared to mdx, while little LTBP4 protein was seen in wild-type and hLTBP4 muscle in the absence of injury or muscular dystrophy (FIG. 11B).

The results showed that there is an increase in inflammatory cell infiltrate in the muscle of hLTBP4/mdx mice versus mdx mice. The results also showed that hLTBP4/mdx muscle possessed increased cleaved LTBP4 protein relative to mdx muscle.

The disclosed subject matter has been described with reference to various specific and preferred embodiments and techniques. It should be understood, however, that many variations and modifications may be made while remaining within the spirit and scope of the disclosed subject matter. All references cited herein are hereby incorporated by reference in their entireties, or to the extent that they provide relevant disclosure, as would be ascertained from context.

What is claimed is:

1. A method of treating a patient having a transforming growth factor beta (TGFβ) superfamily protein-related disease, comprising administering a therapeutically effective amount of an agent that modulates proteolysis of latent TGFβ binding protein 4 (LTBP4) to a patient in need thereof.

2. A method of delaying onset or preventing a transforming growth factor beta (TGFβ) superfamily protein-related disease, comprising administering an effective amount of an agent that modulates proteolysis of latent TGFβ binding protein 4 (LTBP4) to a patient in need thereof.

3. The method of claim 1 or claim 2 wherein the patient suffers from a disease selected from the group consisting of Duchenne Muscular Dystrophy, Limb Girdle Muscular Dystrophy, Becker Muscular Dystrophy, myopathy, cystic fibrosis, pulmonary fibrosis, cardiomyopathy, acute lung injury, acute muscle injury, acute myocardial injury, radiation-induced injury and colon cancer.

4. The method of any one of claims 1-3 wherein the agent is selected from the group consisting of an anti-LTBP4 antibody and a peptide.

5. The method of any one of claims 1-4 further comprising administering an effective amount of a second agent, wherein the second agent is selected from the group consisting of a modulator of an inflammatory response, a promoter of muscle growth, a chemotherapeutic agent, and a modulator of fibrosis.

6. A method of treating a patient having a transforming growth factor beta (TGFβ) superfamily protein-related disease, comprising administering to the patient a therapeutically effective amount of an agent that upregulates the activity of latent TGFβ binding protein 4 (LTBP4).

7. A method of delaying onset or preventing a transforming growth factor beta (TGFβ) superfamily protein-related disease, comprising administering to the patient an effective amount of an agent that upregulates the activity of latent TGFβ binding protein 4 (LTBP4).

8. The method of claim 6 or claim 7 wherein LTBP4 interacts with a TGFβ superfamily protein.

9. The method of any one of claim 8 wherein the TGFβ superfamily protein is selected from the group consisting of TGFβ, a growth and differentiation factor (GDF), activin, inhibin, and a bone morphogenetic protein.

10. The method of claim 9 wherein the GDF is myostatin.

11. The method of any one of claims 6-10 wherein the agent is selected from the group consisting of a peptide, an antibody and a polynucleotide capable of expressing a protein having LTBP4 activity.

12. The method of claim 11 wherein the agent is the peptide of claim 21.

13. The method of claim 11 wherein the agent is the antibody of claim 20.

14. The method of claim 11 wherein the polynucleotide is contained in a vector.

15. The method of claim 14 wherein the vector is a viral vector.

16. The method of claim 15 wherein the viral vector is selected from the group consisting of a herpes virus vector, an adeno-associated virus (AAV) vector, an adeno virus vector, and a lentiviral vector.

17. The method of claim 16 wherein the AAV vector is recombinant AAV9.

18. The method of any one of claims 6-17 wherein the patient has a disease selected from the group consisting of Duchenne Muscular Dystrophy, Limb Girdle Muscular Dystrophy, Becker Muscular Dystrophy, myopathy, cystic fibrosis, pulmonary fibrosis, cardiomyopathy, acute lung injury, acute muscle injury, acute myocardial injury, radiation-induced injury, and colon cancer.

19. The method of any one of claims 6-18 further comprising administering an effective amount of a second agent, wherein the second agent is selected from the group consisting of a modulator of an inflammatory response, a promoter of muscle growth, a chemotherapeutic agent and a modulator of fibrosis.

20. An isolated antibody that specifically binds to a peptide comprising the sequence set forth in SEQ ID NO: 5.

21. A peptide comprising the sequence as set out in any one of SEQ ID NOs: 2-5, or a peptide that is at least 70% identical to the sequence as set out in SEQ ID NOs: 2-5 that retains an ability to act as a substrate for a protease.

22. A pharmaceutical formulation comprising an effective amount of the antibody of claim 20 or the peptide of claim 21, and a pharmaceutically acceptable carrier or diluent.

23. A kit comprising a therapeutically effective amount of the antibody of claim 20 or the peptide of claim 21, a pharmaceutically acceptable carrier or diluent and instructions for use.

24. The formulation of claim 22 or the kit of claim 23, further comprising an effective amount of a second agent, wherein the second agent is selected from the group consisting of a modulator of an inflammatory response, a promoter of muscle growth, a chemotherapeutic agent and a modulator of fibrosis.
